(19) United States
(12) Patent Application Publication
(10) Pub. No.: US 2020/0263258 A1
(43) Pub. Date: Aug. 20, 2020

(54) ASSESSING AND TREATING MAMMALS HAVING POLYPS

(71) Applicant: Mayo Foundation for Medical Education and Research, Rochester, MN (US)

(72) Inventors: Lisa A. Boardman, Rochester, MN (US); Brooke R. Druliner, Rochester, MN (US)

(73) Assignee: Mayo Foundation for Medical Education and Research, Rochester, MN (US)

(21) Appl. No.: 16/791,667

(22) Filed: Feb. 14, 2020

Related U.S. Application Data
(60) Provisional application No. 62/806,671, filed on Feb. 15, 2019.

(51) Int. Cl. C12Q 1/6886; A61K 31/519; A61K 31/513; A61K 31/282

(52) U.S. Cl. CPC C12Q 1/6886; A61K 31/519; C12Q 2600/154; A61K 31/282; C12Q 2600/158; A61K 31/513

(57) ABSTRACT

This document relates to methods and materials for assessing and/or treating mammals (e.g., humans) having one or more polyps (e.g., one or more colon polyps). For example, this document provides methods and materials for determining if a polyp (e.g., a polyp within a mammal having one or more polyps) is likely to recur and/or likely to progress to a cancer. This document also provides methods and materials for treating a mammal having one or more polyps.

### CROSS-REFERENCE TO RELATED APPLICATIONS

This application claims the benefit of U.S. Provisional Application Ser. No. 62/806,671, filed Feb. 15, 2019. The disclosure of the prior application is considered part of (and is incorporated by reference in) the disclosure of this application.

### STATEMENT REGARDING FEDERAL FUNDING

This invention was made with government support under CA170357 awarded by the National Institutes of Health. The government has certain rights in the invention.

### BACKGROUND

### 1. Technical Field

This document relates to methods and materials for assessing and/or treating mammals (e.g., humans) having one or more polyps (e.g., one or more colon polyps). For example, methods and materials provided herein can be used for determining if a polyp (e.g., a polyp within a mammal having one or more polyps) is likely to recur and/or likely to progress to a cancer. This document also provides methods and materials for treating a mammal having one or more polyps.

### 2. Background Information

Colorectal cancer (CRC) develops through progressive accumulation of alterations beginning with abnormal growth of the colon epithelium, which over time can transform to an adenomatous polyp and then cancer (Fearon et al., Cell 61:759-767 (1990)). During the twenty years since adoption of colonoscopy screening, physicians have been able to detect and remove polyps, the precursor lesion for CRC (Citarda et al., Gut 48:812-815 (2001); and Markowitz et al., CA Cancer J. Clin. 47:93-112 (1997)). The majority of CRC arises through transformation of an adenomatous polyp, but only 5% of those polyps progress to cancer (Church, Dis. Colon. Rectum 47:481-485 (2004); Heitman et al., Clin. Gastroenterol. Hepatol. 7:1272-1278 (2009); Martinez et al., Gastroenterology 120:1077-1083 (2001); and Winawer et al., N. Engl. J Med. 328:901-906 (1993)). While colonoscopy allows the detection and subsequent histological evaluation of polyps, those diagnostics fall short of defining features that identify a polyp that is more likely to progress to cancer rather than stay suspended in its premalignant phase.

### SUMMARY

Currently, determining whether a polyp may transform to cancer can be made based on the polyp's size, degree of dysplasia, and histology. In some cases, timing for surveillance colonoscopy can be based on these pathological characteristics. Up to 48% of patients who have complete excision (polypectomy) of an advanced polyp, which is characterized by the presence of villous histology, high grade dysplasia and/or size greater than 1 cm, will have a recurrence of the index polyp in spite of complete resection of the polyp (Laiyemo A O, et al. Digestion. 2013; 87(3):141-6). Even after polypectomy, the risk for the development of invasive CRC is increased 5 fold in post-polypectomy patients who present with an adenomatous polyp greater than 10 mm in size; 7.4 fold if the polyp had villous features; 13 fold with high grade dysplasia; and 4 fold if there were three synchronous polyps (Fairley K J, et al. Clin Transl Gastroenterol. 2014; 5:e64). Similarly, the United States Multisociety Task Force (USMSTF) guidelines have a sensitivity of 59-81% and specificity of 43-58% to predict risk for subsequent metachronous advanced adenomas and results in imprecise overuse and underuse of colonoscopy surveillance (Martinez M E, et al. Gastroenterology. 2001; 120(5): 1077-83). There is a need to be able to identify which polyps are likely to recur and/or likely to progress to cancer, and to offer high-risk patients early therapy, while sparing low risk patients from the risk of toxicity from therapeutic intervention.

This document relates to methods and materials for assessing and/or treating mammals (e.g., humans) having one or more polyps (e.g., one or more colon polyps). For example, this document provides methods and materials for determining if a polyp (e.g., a polyp within a mammal having one or more polyps) is likely to recur and/or likely to progress to a cancer. In some cases, a molecular profile of a polyp can be used to determine if that polyp is likely to recur and/or likely to progress to a cancer. This document also provides methods and materials for treating a mammal having one or more polyps (e.g., one or more colorectal polyps).

As demonstrated herein, the molecular profile of a polyp can distinguish whether that polyp is a benign polyp or a malignant polyp. Whole genome sequencing (WGS), RNA-sequencing (RNA-seq), and reduced representation bisulfite sequencing (RRBS) were used to determine a molecular profile of over 90 cancer adjacent polyps (CAPs) and cancer free polyps (CFPs) from 31 patients. CAPs can have more genetic mutations (e.g., somatic variants), altered polypeptide expression, and hypermethylation of nucleic acid sequences compared to CFPs. APC was significantly mutated in both polyp groups, but mutations in TP53, FBXW7, PIK3CA, KIAA1804 and SMAD2 were exclusive to CAPs. Expression changes were found between CAPs and CFPs in GREM1, IGF2, CTGF, and PLAU, and both expression and methylation alterations in FES and HES1. Integrative analyses revealed 124 genes with alterations in at least two platforms, and ERBB3 and E2F8 showed aberrations specific to CAPs across all platforms. These findings provide a resource of molecular distinctions between polyps with and without cancer, which have the potential to enhance the diagnosis, risk assessment and management of polyps.

Having the ability to determine risk whether a polyp in patients having one or more polyps (e.g., one or more colon polyps) is likely to recur and/or likely to progress to a cancer provides a unique and unrealized opportunity to initiate early therapy, rather than waiting to treat patients having one or more polyps (e.g., one or more colon polyps) until after a cancer has developed.

In general, one aspect of this document features methods for treating a mammal having one or more colon polyps. The methods can include, or consist essentially of, identifying at least one polyp from a mammal having one or more colon polyps as having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG, RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, where the one or more modifications can be selected from the group consisting of a somatic variation in a nucleic acid sequence, altered expression of a nucleic acid sequence, altered methylation of a nucleic acid sequence, and combinations thereof; and administering a colon polyp treatment to the mammal under conditions where the number of colon polyps within the mammal is reduced. The mammal can be a human. The molecular profile can include one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence; increased expression of one or more of a CXCL5, a GREM1, an IGF2, a CTGF, a PLAU, and an E2F8 nucleic acid sequence; reduced expression of an E2F8 nucleic acid sequence; and hypermethylation of one or more a FES, a HES1, an ERBB3, and an E2F8 nucleic acid sequence. The molecular profile can include one or more somatic variations in a TP53, a FBXW7, a PIK3CA, a KIAA1804, a SMAD2, and a SMAD4 nucleic acid sequence; increased expression of a CXCL5, a GREM1, an IGF2, a CTGF, and a PLAU nucleic acid sequence; reduced expression of an ERBB3 nucleic acid sequence; and hypermethylation of a FES, a HES1, an ERBB3, and an E2F8 nucleic acid sequence can be identified as being likely to recur and/or likely to progress to a cancer. The colon polyp treatment can include removal of one or more polyp(s) in addition to the polyp having said molecular profile. The method can include selecting the mammal for more frequent cancer screening (e.g., more frequent than was performed previously on the mammal). The method also can include performing the more frequent cancer screening. The cancer screening can be colonoscopy, barium enema x-rays, digital rectal examinations, or combinations thereof.

In another aspect, this document features methods for treating a mammal having one or more colon polyps. The methods can include, or consist essentially of, administering a colon polyp treatment to a mammal identified as having at least one polyp having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG, RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, where the one or more modifications can be selected from the group consisting of a somatic variation in a nucleic acid sequence, altered expression of a nucleic acid sequence, altered methylation of a nucleic acid sequence, and combinations thereof. The mammal can be a human. The molecular profile can include one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence; increased expression of one or more of a CXCL5, a GREM1, an IGF2, a CTGF, a PLAU, and an E2F8 nucleic acid sequence; reduced expression of an E2F8 nucleic acid sequence; and hypermethylation of one or more a FES, a HES1, an ERBB3, and an E2F8 nucleic acid sequence. The molecular profile can include one or more somatic variations in a TP53, a FBXW7, a PIK3CA, a KIAA1804, a SMAD2, and a SMAD4 nucleic acid sequence; increased expression of a CXCL5, a GREM1, an IGF2, a CTGF, and a PLAU nucleic acid sequence; reduced expression of an ERBB3 nucleic acid sequence; and hypermethylation of a FES, a HES1, an ERBB3, and an E2F8 nucleic acid sequence can be identified as being likely to recur and/or likely to progress to a cancer. The colon polyp treatment can include removal of one or more polyp(s) in addition to the polyp having said molecular profile. The method can include selecting the mammal for more frequent cancer screening (e.g., more frequent than was performed previously on the mammal). The method also can include performing the more frequent cancer screening. The cancer screening can be colonoscopy, barium enema x-rays, digital rectal examinations, or combinations thereof.

In another aspect, this document features methods for treating a mammal having one or more colon polyps. The methods can include, or consist essentially of, identifying at least one polyp from a mammal having one or more colon polyps as having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, where the one or more modifications can be selected from the group consisting of a somatic variation in a nucleic acid sequence, altered expression of a nucleic acid sequence, altered methylation of a nucleic acid sequence, and combinations thereof; administering a colon polyp treatment to the mammal under conditions where the number of colon polyps within the mammal is reduced; and administering a cancer treatment to the mammal. The mammal can be a human. The molecular profile can include one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence; increased expression of one or more of a CXCL5, a GREM1, an IGF2, a CTGF, a PLAU, and an E2F8 nucleic acid sequence; reduced expression of an E2F8 nucleic acid sequence; and hypermethylation of one or more a FES, a HES1, an ERBB3, and an E2F8 nucleic acid sequence. The molecular profile can include one or more somatic variations in a TP53, a FBXW7, a PIK3CA, a KIAA1804, a SMAD2, and a SMAD4 nucleic acid sequence; increased expression of a CXCL5, a GREM1, an IGF2, a CTGF, and a PLAU nucleic acid sequence; reduced expression of an ERBB3 nucleic acid sequence; and hypermethylation of a FES, a HES1, an ERBB3, and an E2F8 nucleic acid sequence. The colon polyp treatment can include removal of the polyp(s). The cancer treatment can include administering a cancer drug to the mammal. The cancer drug can be capecitabine, fluorouracil, oxaliplatin, leucovorin, avastin, cetuximab, pembrolizumab, and combinations thereof.

In another aspect, this document features methods for treating a mammal having one or more colon polyps. The methods can include, or consist essentially of, administering a colon polyp treatment to a mammal identified as having at least one polyp having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG, RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, where the one or more modifications can be selected from the group consisting of a somatic variation in a nucleic acid sequence, altered expression of a nucleic acid sequence, altered methylation of a nucleic acid sequence, and combinations thereof, and administering a cancer treatment to the mammal. The mammal can be a human. The molecular profile can include one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence; increased expression of one or more of a CXCL5, a GREM1, an IGF2, a CTGF, a PLAU, and an E2F8 nucleic acid sequence; reduced expression of an E2F8 nucleic acid sequence; and hypermethylation of one or more a FES, a HES1, an ERBB3, and an E2F8 nucleic acid sequence. The molecular profile can include one or more somatic variations in a TP53, a FBXW7, a PIK3CA, a KIAA1804, a SMAD2, and a SMAD4 nucleic acid sequence; increased expression of a CXCL5, a GREM1, an IGF2, a CTGF, and a PLAU nucleic acid sequence; reduced expression of an ERBB3 nucleic acid sequence; and hypermethylation of a FES, a HES1, an ERBB3, and an E2F8 nucleic acid sequence. The colon polyp treatment can include removal of the polyp(s). The cancer treatment can include administering a cancer drug to the mammal. The cancer drug can be capecitabine, fluorouracil, oxaliplatin, leucovorin, avastin, cetuximab, pembrolizumab, and combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1F show a cancer-adjacent polyp (CAP) and cancer free polyp (CFP) model, and show that Whole Genome Sequencing can distinguish CAP from CFP tissues. FIG. 1A shows CAP cases that are represented schematically. FIG. 1B shows CFP cases that are represented schematically. CAP cases include matched, distant normal colon epithelium, the polyp (residual polyp of origin) and the corresponding cancer that arose from the polyp (CRC RPO+). CFP cases include matched, distant normal colon epithelium and the villous adenoma (polyp). CFP cases are those that have had polyps present and removed that have not gone on to cancer. All polyps cases used in the study were matched by histology and degree of dysplasia-villous adenomas with low-grade dysplasia. The anatomical location in the colon of the polyp and cancer in the diagram serves only as an exemplar case as polyp or tumor location has no impact on the likelihood of finding a CAP or CFP case. Hematoxylin and eosin (H&E) staining showing the specific histologic features of the (FIG. 1A) distant normal colon, CAP, CRC RPO+ and (FIG. 1B) distant normal colon and CFP. FIG. 1C shows mutations that were significantly different between the CAPs and CFPs were identified by k-nearest neighbors algorithm. The x-axis shows the number of patients in which the gene is variant for CFP tissues, the y-axis is CRC RPO+(tumor tissue), and the z-axis is CAP tissues. FIG. 1D shows the somatic mutation frequency of 10 genes found to be commonly mutated in CRC by the TCGA. The mutation frequencies of these genes from the CAPs and CFPs were compared. FIG. 1E shows a heatmap and clustering of significantly mutated genes determined by MutSig algorithm for CAPs vs. PBL, normal colon; CFPs vs. PBL, normal colon; and Cancer vs. PBL, normal colon. Red indicates a correlation of 1. FIG. 1F shows the mean quantity of single nucleotide variants (SNVs) in CAP tissues and CFP tissues. The y-axis is number of SNVs, and the x-axis is the genomic feature, and total of all features in the far right bar plots.

FIG. 2 shows the somatic mutation frequency of 10 genes found to be commonly mutated in CRC by the TCGA. The mutation frequencies of these genes for the CAPs, cancer tissues (of CAPs), and CFPs were compared.

FIG. 3 shows the presence of mutations in 10 genes on a patient-by-patient basis. The top panel shows genes having mutations in polyp tissues of CAP patients. The bottom panel shows genes having mutations in cancer tissues of CAP patients.

FIGS. 4A-4B show features of INDELS and Structural Variants between CAPs and CFPs. FIG. 4A shows the quantity of INDELs in CAP tissues and CFP tissues. FIG. 4B shows the quantity of Structural Variants in CAP tissues and CFP tissues. The y-axis is number of INDELs, Structural Variants, or CNV; the x-axis is the genomic feature, and total of all features in the far right bar plots.

FIG. 5 shows aneuploidy percentages between CAPs and CFPs. Boxplot of percentage of aneuploidy for CAP and CFP polyp tissues. Mean CNV % listed below label on x-axis.

FIG. 6 shows tissues from the same patient cluster together on the basis on CNV. Most CNVs are shared from different tissues of the same patient, even with common CNVs across all patients/samples removed. The distances between samples are calculated by −log p-value of hypergeometric test. Each patient tissue is listed on the top axis, each color represents a new patient with set of tissues, and each line represents a different tissue.

FIG. 7 shows whole genome plots of aneuploidy for CAP and CFP tissues. From top to bottom, CAP normal epithelium, villous polyp, cancer; and CFP normal epithelium, and villous polyp. Y-axis is the read coverage and x-axis is the bin index.

FIG. 8 shows a heatmap of CNV analysis and hierarchical clustering by tissue type. Deletions (blue) or duplications (red) are indicating for each sample. The alternating grey and black bars at the bottom represent the span of each chromosome. Samples are grouped together by similarity in pairwise CNV using UPGMA. The bottom grid is the summary of chromosomes with the most recurrent changes for the cancer, CAP and CFP (top to bottom). Chromosomes with significant changes are highlighted in olive green for cancer samples, yellow for CAPs, and purple for CFPs.

FIGS. 9A-9G show gene expression determined by RNA-seq distinguishes CAP from CFP tissues. FIG. 9A shows a dendrogram based on average distance of the whole transcriptome between the CAP tissues and CFP tissues. Each patient ID beginning with the letter A is shown. FIG. 9B shows a volcano plot showing all differentially expressed genes between the CAP and CFP tissues. The x-axis is the log of the fold change in expression, and the y-axis is the log of the FDR between CAP and CFP tissues. Green dots are genes that have a fold change >2, and FDR >0.1. For a list of genes that are above these thresholds see Table 6. FIG. 9C is a boxplot of CXCL5 gene expression for CAPs and CFPs polyp tissues. Y-axis is the log₂ of the gene counts. The inset shows the boxplots for the normal and polyp tissues from CAP patients (left) and CFP patients (right) for CXCL5, showing the relative change between normal and polyp. FIGS. 9D-9G contains similar boxplots GREM1 (FIG. 9D), IGF2 (FIG. 9E), CTGF (FIG. 9F), and PLAU (FIG. 9G).

FIGS. 10A-10C shows differential hypermethylated regions distinguish CAP from CFP tissues. FIG. 10A contains a boxplot showing the total CpG mean value of all examined by RRBS for CAP and CFP tissues. FIG. 10B contains a scatterplot showing the differentially methylated regions between CAPs and CFPs. The x-axis is the log of the area under the curve (AUC), and the y-axis is the log of the FDR between CAP and CFP tissues. Red dots are genes that have an AUC >0.85, and p-value >0.05. For a list of genes that are above these thresholds and colored red see Table 13. FIG. 10C contains boxplots showing the CpG mean (left plots) and normalized gene expression values (right plots) for FES (top plots) and HES1 (bottom plots) between CAP and CFP tissues. The bottom of the boxplots for the CpG mean plots shows the gene diagram, with the red box illustrating the location of the hypermethylated CpG islands, with scales shown.

FIGS. 11A-11B show that integration of multiple platforms revealed a 124 gene panel, which distinguishes CAP from CFP tissues. FIG. 11A shows the overlap between significantly mutated genes determined by WGS, differentially expressed genes by RNA-seq and differentially methylated regions by RRBS between CAP and CFP tissues. The red highlighted area showing the two genes that have a genetic variant, altered expressed and altered expression between the CAPs and CFPs. FIG. 11B contains boxplots showing the CpG Mean (left plots) and normalized gene expression (right plots) for the ERBB3 (top plots) and E2F8 (bottom plots) genes, which also have SNVs present. The bottom of the boxplots for the CpG mean plots shows the gene diagram, with the red box illustrating the location of the hypermethylated CpG islands, with scales shown.

FIG. 12 contains Table 1 showing patients and corresponding tissues and sequencing platforms applied, with annotation on tissue type and clinical behavior.

FIG. 13 contains Table 3 showing pathway enrichment by Kyoto Encyclopedia of Genes and Genomes (KEGG) for genes that have differential somatic variants between CAP and CFP tissues.

FIG. 14 contains Table 4 showing genes with significant expression changes between CAP and CFPs (2,452 genes).

FIG. 15 contains Table 6 showing genes with significant expression changes (FDR<0.1 and fold change >2) between CAP and CFPs.

FIG. 16 contains Table 7 showing gene ontology terms and pathways enriched by differentially expressed genes between CAP and CFP polyps using DAVID (total gene input 2,452, from Table 4).

FIG. 17 contains Table 8 showing gene ontology and pathways enriched by differentially expressed genes between CAP and CFP polyps with FDR<0.1 and fold change >2 (102 gene input, from Table 6) using DAVID.

FIG. 18 contains Table 9 showing gene ontology terms and proteins enriched by differentially expressed genes between CAP and CFP polyps using PANTHER.

FIG. 19 contains Table 10 showing functional annotation clustering defined by differentially expressed genes between CAP and CFP polyps using DAVID (total gene input 2,452, from Table 4).

FIG. 20 contains Table 11 showing functional annotation clustering defined by differentially expressed genes between CAP and CFP polyps with FDR<0.1 and fold change >2 (102 gene input, from Table 6) using DAVID.

FIG. 21 contains Table 12 showing 30 genes with significant hypermethylation at Differentially Methylated Regions between CAP and CFPs and with a Fold Change >20.

FIG. 22 contains Table 13 showing 87 genes with significant differentially methylated regions between CAP and CFPs and with AUC>0.85.

FIG. 23 contains Table 15 showing patients, tissue types, assay types, and accession numbers.

FIGS. 24A-24C show genetics and gene expression vary in polyps based on recurrence or association with cancer. FIG. 24A shows a comparison of the mutation burden in the polyp tissues between POP categories. FIG. 24B shows comparisons of Copy Number Variation in the polyp tissues between POP categories. ** represents a statistically significant difference, which were seen between the non-recurrent polyps compared to the polyps associated with CRC. POP-NR, n=7; POP-R, n=7; POP-CRC, n=16. FIG. 24C contains volcano plots showing all differentially expressed genes between pairwise comparisons of POP categories; the plots from left to right are the expression differences in the polyp tissues between: POP-NR vs POP-R, POP-NR vs POP-CRC, POP-R vs POP-CRC. The x-axis is the log fold change in expression, and the y-axis is the log of the p-value between polyps in the different POP categories. Green dots are genes that have a fold change >2, and p-value <0.01. For expression data: POP-NR, n=31; POP-R, n=31; POP-CRC, n=69.

### DETAILED DESCRIPTION

This document provides methods and materials for assessing and/or treating mammals (e.g., humans) having one or more polyps (e.g., one or more colon polyps). For example, the methods and materials provided herein can be used for determining if a polyp (e.g., a polyp within a mammal having one or more polyps) is likely to recur and/or likely to progress to a cancer. In some cases, a molecular profile of a polyp can be used to determine if that polyp may be likely to recur and/or progress to a cancer. For example, a sample (e.g., a polyp sample) obtained from a mammal having one or more polyps can be assessed to determine if a polyp is likely to recur and/or likely to progress to a cancer based, at least in part, on the molecular profile of the polyp. When a polyp sample obtained from a mammal having one or more polyps is determined to be a polyp that is likely to recur and/or likely to progress to a cancer based, at least in part, on the molecular profile of the polyp, it is likely that polyps remaining in the mammal can have the same molecular profile as the polyp sample and may also be likely to recur and/or likely to progress to a cancer. As described herein, a distinct molecular profile can be present in a polyp that is likely to recur and/or likely to progress to a cancer (e.g., as compared to a molecular profile that can be present in a polyp that is not likely to recur and/or likely to progress to a cancer). This document also provides methods and materials for treating a mammal having one or more polyps (e.g., one or more colorectal polyps). For example, a treatment for a mammal having one or more polyps can be selected based, at least in part, on the molecular profile of the mammal's polyp(s) as described herein.

Any type of mammal can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. In some cases, the mammal can be a human. In some cases, a mammal can be a mammal having one or more polyps. In some cases, a mammal can have one or more polyp disorders (e.g., one or more hereditary polyp disorders). Examples of hereditary polyp disorders can include, without limitation, Lynch syndrome, familial adenomatous polyposis (FAP), Gardner's Syndrome, MYH-associated polyposis (MAP), Peutz-Jeghers Syndrome, Juvenile Polyposis Syndrome, PTEN Hamartomata Tumor Syndrome, Hereditary Mixed Polyposis Syndrome, and Serrated Polyposis Syndrome. For example, a mammal having one or more polyps can be assessed for whether a polyp may be likely to recur and/or may be likely to progress to a cancer, and can be treated with one or more interventions as described herein.

A mammal having one or more polyps can have any type of polyp(s). In some cases, a polyp can be a non-neoplastic polyp (e.g., hyperplastic polyps and inflammatory polyps). In some cases, a polyp can be a neoplastic polyp (e.g., adenomas and serrated polyps).

A mammal having one or more polyps can have polyp(s) in any location within the mammal. Examples of locations within a mammal that can have one or more polyps that can be assessed and/or treated as described herein can include, without limitation, colon, breasts, stomach, small intestine, urinary tract, ovaries, skin, bones, abdomen, lips, gums, nasal cavity, lung, pancreas, and gall bladder. In some cases, a polyp that is assessed and/or treated using the methods and materials described herein can be a colon polyp (e.g., a colorectal polyp).

A mammal having one or more polyps can have any size polyp(s). In some cases, a polyp can be from about 0.5 mm to about 80 mm (e.g., from about 0.5 mm to about 70 mm, from about 0.5 mm to about 60 mm, from about 0.5 mm to about 50 mm, from about 0.5 mm to about 40 mm, from about 0.5 mm to about 30 mm, from about 0.5 mm to about 20 mm, from about 0.5 mm to about 10 mm, from about 1 mm to about 80 mm, from about 5 mm to about 80 mm, from about 10 mm to about 80 mm, from about 20 mm to about 80 mm, from about 30 mm to about 80 mm, from about 40 mm to about 80 mm, from about 50 mm to about 80 mm, from about 60 mm to about 80 mm, from about 70 mm to about 80 mm, from about 5 mm to about 60 mm, from about 20 mm to about 50 mm, from about 30 mm to about 40 mm, from about 10 mm to about 30 mm, from about 30 mm to about 50 mm, from about or from about 50 mm to about 70 mm) in size (e.g., across its diameter or longest dimensions).

A mammal having one or more polyps can have any number of polyps. In some cases, a mammal can have from about one polyp to thousands of polyps. In some cases, a mammal can have two or more polyps (e.g., two three, four, five, six, seven, eight, nine, 10, 11, 12, 13, 14, 15, 16, 17, 18, 19, 20, or more polyps).

In some cases, a mammal that is assessed and/or treated as described herein can be identified as having one or more polyps. Any appropriate method can be used to identify a mammal as having one or more polyps. In some cases, imaging techniques such as using a flexible tube with a light and camera attached to it to visualize internal organs (e.g., colonoscopy, endoscopy, and sigmoidoscopy), computerized tomography (CT) scanning (e.g., CT colonography), and x-ray techniques (e.g., barium enema x-ray techniques) can be used to identify a mammal as having one or more polyps. In some cases, laboratory tests such as stool-based tests (e.g., checking for the presence of blood in the stool and/or assessing your stool DNA) can be used to identify a mammal as having one or more polyps. In some cases, physical examinations (e.g., digital rectal examinations) can be used to identify a mammal as having one or more polyps.

Once identified as having one or more polyps, a mammal can be assessed to determine whether a polyp may be likely to recur and/or may be likely to progress to a cancer. For example, a sample (e.g., a polyp sample) obtained from the mammal having one or more polyps can be assessed whether a polyp may be likely to recur and/or may be likely to progress to a cancer. As described herein, a sample obtained from a mammal having one or more polyps can be used to determine a molecular profile of a polyp, and can be used to determine whether the polyp may be likely to recur and/or may be likely to progress to a cancer.

Any appropriate sample from a mammal (e.g., a human) having one or more polyps can be assessed as described herein. In some cases, a sample can be a biological sample. For example, a sample can be a polyp sample. In some cases, a polyp sample can contain at least a portion of a polyp. In some cases, a polyp sample can contain one or more polyps. In some cases, a sample can contain one or more biological molecules (e.g., nucleic acids such as DNA and RNA, proteins, carbohydrates, lipids, hormones, metabolites, and/or microbial/viral species. In some cases, a biological sample can be one or more cells (e.g., cultured cells such as cell lines and organoids such as 2D or 3D patient-derived organoids). Examples of samples that can be assessed as described herein include, without limitation, tissue samples (e.g., colon tissue samples, rectum tissue samples, and skin tissue samples), stool samples, cellular samples (e.g., buccal samples), and fluid samples (e.g., blood, serum, plasma, urine, and saliva). A biological sample can be a fresh sample or a fixed sample. In some cases, a biological sample can be a processed sample (e.g., an embedded sample such as a paraffin or OCT embedded sample, a processed to isolate or extract one or more biological molecules). For example, a colon tissue sample and/or a rectum tissue sample can be obtained from a mammal having one or more polyps and can be assessed to determine if a polyp within the mammal may be likely to recur and/or may be likely to progress to a cancer based, at least in part, on a molecular profile of the polyp.

A molecular profile described herein can include a panel of biomarkers. A panel of biomarkers can include any number of biomarkers. For example, a panel of biomarkers can include any two or more (e.g., two, three, five, eight, 10, 12, 15, 17, 20, 25, 30, 40, 50, 60, 70, 80, 90, 100, 110, 120, 130, or more) biomarkers. A biomarker can be any type of biological molecule. Examples of biological molecules that can be used as a biomarker in a molecular profile described herein can include, without limitation, nucleic acid sequences, proteins, carbohydrates, lipids, hormones, and microbial/viral species. When a biomarker is a nucleic acid sequence, the nucleic acid sequence can be any appropriate nucleic acid sequence. In some cases, a nucleic acid sequence can encode a polypeptide involved in a cellular pathway such as a Hippo signaling pathway, a TGF-beta signaling pathway, a cAMP signaling pathway, an oxytocin signaling pathway, a Wnt signaling pathway, a signaling pathway regulating pluripotency of stem cells, a cGMP-PKG signaling pathway, and an adherens junction pathway. Examples of nucleic acid sequences that can be used as biomarkers in a molecular profile described herein include, without limitation, E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG RASAL3, HMCN1, TRPV3, CR2, OTOP3, COL13A1, CPLX1, DUSP2, MT1JP NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences. In some cases, a molecular profile described herein can include one or more biomarkers set forth in Table 3, Table 4, Table 6, Table, 12, and/or Table 13. In some cases, a molecular profile can be as described in Example 1. In some cases, a molecular profile can be as described elsewhere (see, e.g., Druliner et al., Scientific REPORTS 8:3161 (2018)).

In some cases, a biomarker (e.g., a biomarker present in a molecular profile of a polyp that is likely to recur and/or likely to progress to a cancer) can be a modified biological molecule. Examples of modified biological molecules that can be used as a biomarker in a molecular profile described herein can include, without limitation, a nucleic acid sequence having one or more somatic variations, a nucleic acid sequence having altered (e.g., increased or decreased) expression (e.g., thereby resulting in altered levels of a polypeptide encoded by that nucleic acid sequence), epigenetics changes such as altered methylation of the nucleic acid sequence, altered transcription factor binding, and changes in nuclear structure (e.g. changes in histone marks and chromatin structural changes). When a biomarker is associated with a polyp that is likely to recur and/or likely to progress to a cancer, the modification can be as compared to a molecular profile that can be present in a sample (e.g., a control sample) from one or more healthy mammals (e.g., healthy humans). Control samples can include, without limitation, cancer free polyps, samples from mammals that do not have cancer, cell lines originating from mammals that do not have cancer, non-tumorigenic cell lines, and organoids originating from mammals that do not have cancer. When a biomarker in a molecular profile described herein is a modified biological molecule, the biological molecule can include at least one (e.g., one, two, three, or more) modification. In some cases, biomarker in a molecular profile described herein is a modified biological molecule, the biological molecule can include at least two (e.g., two, three, or more) modifications. For example, a nucleic acid sequence in a molecular profile described can have one or more somatic variations and can have altered expression. For example, a nucleic acid sequence in a molecular profile described herein can have one or more somatic variations and can have altered methylation. For example, a nucleic acid sequence in a molecular profile described herein can have altered expression and can have altered methylation. In some cases, biomarker in a molecular profile described herein is a modified biological molecule, the biological molecule can include at least three (e.g., three or more) different modifications. For example, a nucleic acid sequence in a molecular profile described herein having at least three different molecular characteristics can have one or more somatic variations, can have altered expression, and can have altered methylation.

In cases where a biomarker is a nucleic acid sequence having one or more somatic variations, a somatic variation can be any appropriate somatic variation. When a somatic variation in a nucleic acid sequence is associated with a polyp that is likely to recur and/or likely to progress to a cancer, the somatic variation can be as compared to a corresponding nucleic acid sequence that can be present in a sample (e.g., a control sample) from one or more healthy mammals (e.g., healthy humans). For example, when a somatic variation is associated with a polyp that is likely to recur and/or likely to progress to a cancer, the somatic variation is typically not observed in a corresponding nucleic acid sequence in a control sample. Examples of somatic variants can include, without limitation, single nucleotide variants (SNVs), insertions, deletions, insertion/deletions (INDELs), copy number variations (CNVs), transposons, and structural variants (SVs). For example, a biomarker included in a molecular profile described herein can include one or more somatic variations in any appropriate nucleic acid sequence. Examples of nucleic acid sequences that can include one or more somatic variations and can be used as biomarkers in a molecular profile described herein include, without limitation, APC, TP53, TTN, KRAS, FBXW7, PIK3CA, CTNNB1, KIAA1804, SMAD2, SMAD4, ERBB3, and E2F8 nucleic acid sequences. In some cases, a molecular profile described herein can include one or more somatic variations in one or more of the nucleic acid sequences set forth in Table 3.

In cases where a biomarker is a nucleic acid sequence having altered expression, the altered expression can be an increase or a decrease in the expression of the nucleic acid sequence. When altered expression of a nucleic acid sequence is associated with a polyp that is likely to recur and/or likely to progress to a cancer, the altered expression can be as compared to an expression level of a corresponding nucleic acid sequence in a sample (e.g., a control sample) from one or more healthy mammals (e.g., healthy humans). For example, when altered expression is an increase in the expression of a nucleic acid sequence, increased expression refers to any level of nucleic acid expression (e.g., any level of a polypeptide encoded by the nucleic acid sequence) that is higher than the median level of expression of a corresponding nucleic acid sequence typically observed in a control sample. For example, when altered expression is a decrease in the expression of a nucleic acid sequence, decreased expression refers to any level of nucleic acid expression (e.g., any level of a polypeptide encoded by the nucleic acid sequence) that is lower than the median level of expression of a corresponding nucleic acid sequence typically observed in a control sample. For example, a biomarker included in a molecular profile described herein can include altered expression of any appropriate nucleic acid sequence. Examples of nucleic acid sequences that can have altered expression and can be used as biomarkers in a molecular profile described herein include, without limitation, CXCL5, GREM1, IGF2, CTGF, PLAU, ERBB3, and E2F8 nucleic acid sequences. In some cases, a molecular profile described herein can include altered expression of one or more of the nucleic acid sequences set forth in Table 4 and Table 6. In some cases, altered expression of a nucleic acid sequence can result in altered (e.g., increased or decreased) levels of a polypeptide encoded by that nucleic acid sequence. For example, a biomarker included in a molecular profile described herein can include altered levels of any appropriate polypeptide. Examples of polypeptides that can have altered polypeptide levels and can be used as biomarkers in a molecular profile described herein include, without limitation, CXCL5, GREM1, IGF2, CTGF, PLAU, ERBB3, and E2F8 polypeptides.

In cases where a biomarker is a nucleic acid sequence having altered methylation, the altered methylation can be an increase in methylation (e.g., hypermethylation) or a decrease in methylation (e.g., hypomethylation). When altered methylation of a nucleic acid sequence is associated with a polyp that is likely to recur and/or likely to progress to a cancer, the altered methylation can be as compared to a level of methylation on a corresponding nucleic acid sequence in a sample (e.g., a control sample) from one or more healthy mammals (e.g., healthy humans). For example, when altered methylation is hypermethylation of a nucleic acid sequence, hypermethylation refers to any level of methylation that is higher than the median level of methylation of a corresponding nucleic acid sequence typically observed on a nucleic acid in a control sample. For example, when altered methylation is hypomethylation of a nucleic acid sequence, hypomethylation refers to any level of methylation that is lower than the median level of methylation of a corresponding nucleic acid sequence typically observed on a nucleic acid in a control sample. For example, a biomarker included in a molecular profile described herein can include altered methylation of any appropriate nucleic acid sequence. Examples of nucleic acid sequences that can have altered methylation and can be used as biomarkers in a molecular profile described herein include, without limitation, FES, HES1, ERBB3, and E2F8 nucleic acid sequences. In some cases, a molecular profile described herein can include altered methylation of one or more of the nucleic acid sequences set forth in Table 12 and Table 13.

Any appropriate method can be used to identify the presence or absence of one or more biomarkers described herein (e.g., one or more biomarkers in a molecular profile described herein). For example, when a biomarker is a nucleic acid sequence having one or more somatic variations, sequencing (e.g., PCR-based sequencing such as Next-Generation PCR-based sequencing and Sanger sequencing), DNA hybridization, and restriction enzyme digestion methods can be used to identify the presence or absence of one or more somatic variations in the nucleic acid sequence. For example, when a biomarker is a nucleic acid sequence having altered expression, immunohistochemistry (IHC) techniques (e.g., immunofluorescence), mass spectrometry techniques (e.g., proteomics-based mass spectrometry assays or targeted quantification-based mass spectrometry assays), western blotting techniques, and quantitative RT-PCR techniques can be used to identify the presence, absence, or level of expression of the nucleic acid sequence. For example, when a biomarker is a nucleic acid sequence having altered methylation, methylation-sensitive high resolution melting (MS-HRM), methylation specific qPCR, bisulfite sequencing (e.g., reduced representation bisulfite sequencing (RRBS) and whole genome bisulfite sequencing (WGBS)) can be used to identify the presence, absence, or level of methylation on the nucleic acid sequence. In some cases, a biomarker can be identified as described in Example 1. In some cases, a biomarker described herein can be identified as described elsewhere (see, e.g., Druliner et al., Scientific REPORTS 8:3161 (2018)).

In some cases, a molecular profile can be used to determine whether a polyp is likely to recur and/or likely to progress to a cancer. A molecular profile that can be used to determine whether a polyp is likely to recur and/or likely to progress to a cancer can include any appropriate biomarkers in the molecular profile. For example, a polyp having a molecular profile including one or more somatic variations in one or more of a APC, a TP53, a TTN, a KRAS, a FBXW7, a PIK3CA, a CTNNB1, a KIAA1804, a SMAD2, a SMAD4, an ERBB3, and/or an E2F8 nucleic acid sequence; having increased expression of one or more of a CXCL5, a GREM1, an IGF2, a CTGF, a PLAU, and/or an E2F8 nucleic acid sequence; having reduced expression of an E2F8 nucleic acid sequence; and having hypermethylation of one or more a FES, a HES1, an ERBB3, and/or an E2F8 nucleic acid sequence can be identified as being likely to recur and/or likely to progress to a cancer. In some cases, a polyp having a molecular profile including one or more somatic variations in a TP53, a FBXW7, a PIK3CA, a KIAA1804, a SMAD2, and a SMAD4 nucleic acid sequence; having increased expression of a CXCL5, a GREM1, an IGF2, a CTGF, and a PLAU nucleic acid sequence; having reduced expression of an ERBB3 nucleic acid sequence; and having hypermethylation of a FES, a HES1, an ERBB3, and an E2F8 nucleic acid sequence can be identified as being likely to recur and/or likely to progress to a cancer.

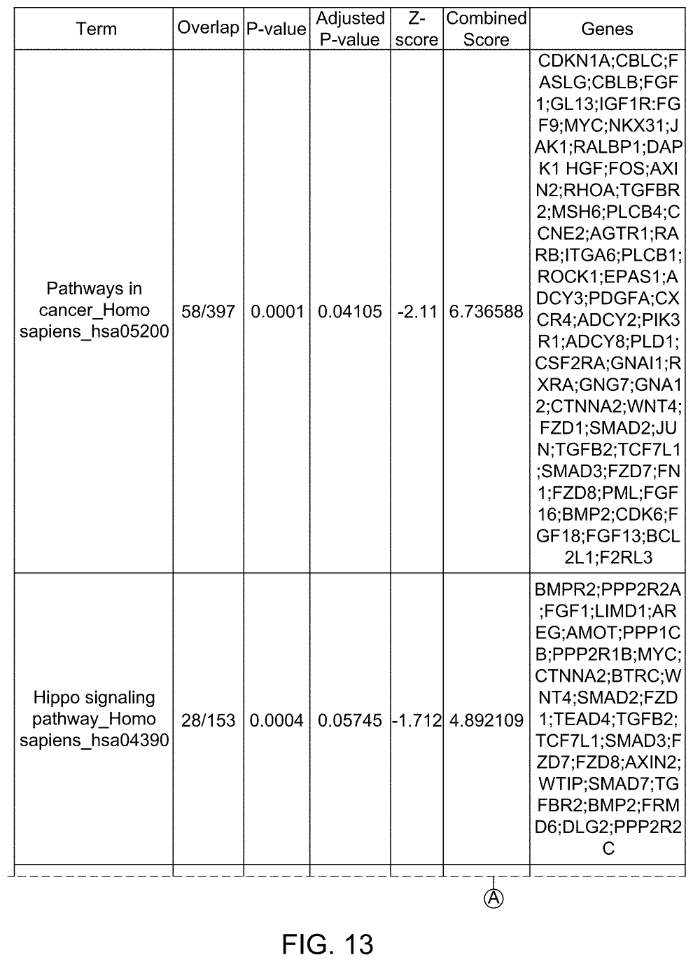

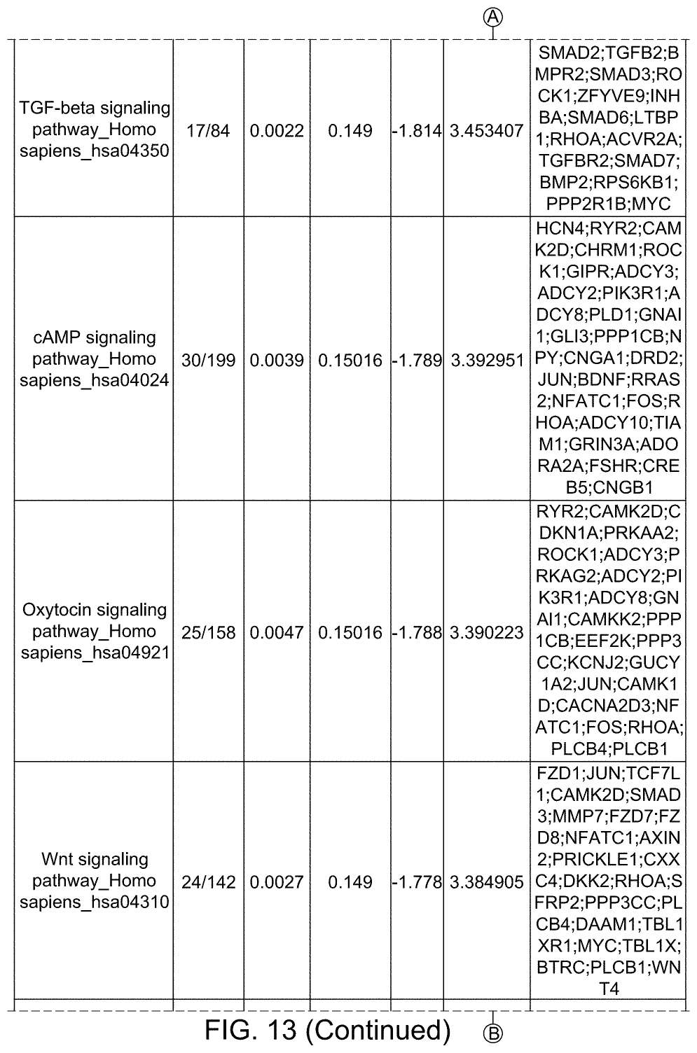

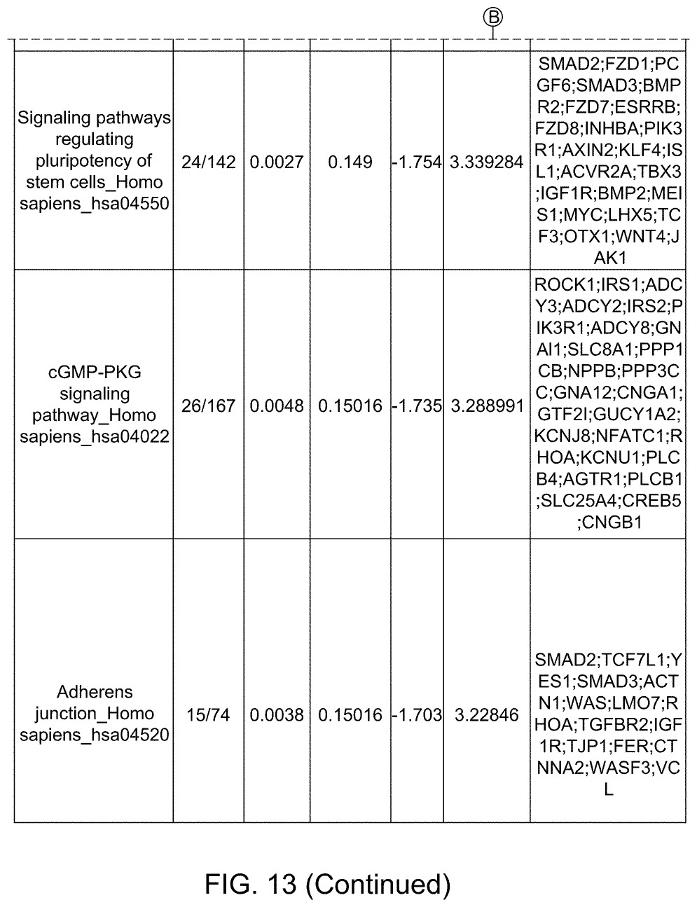

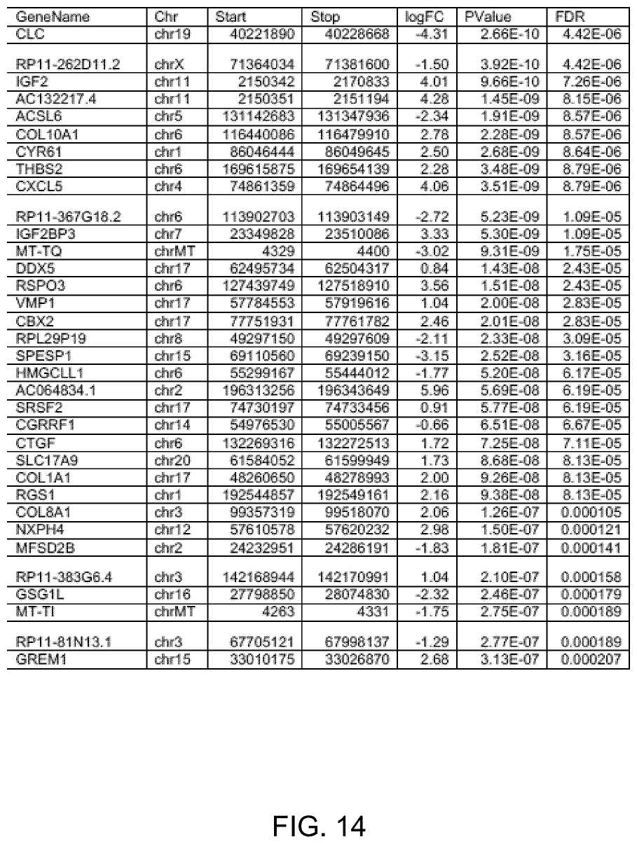

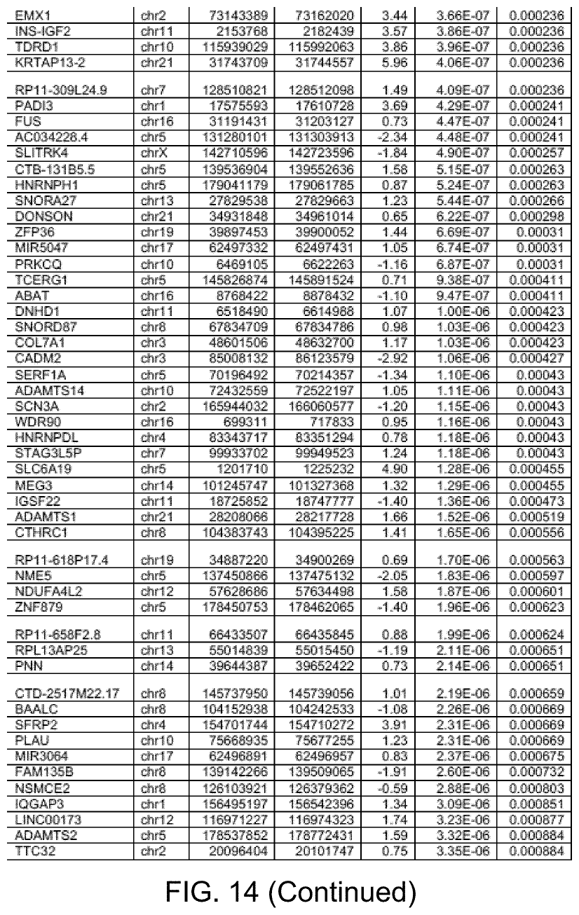

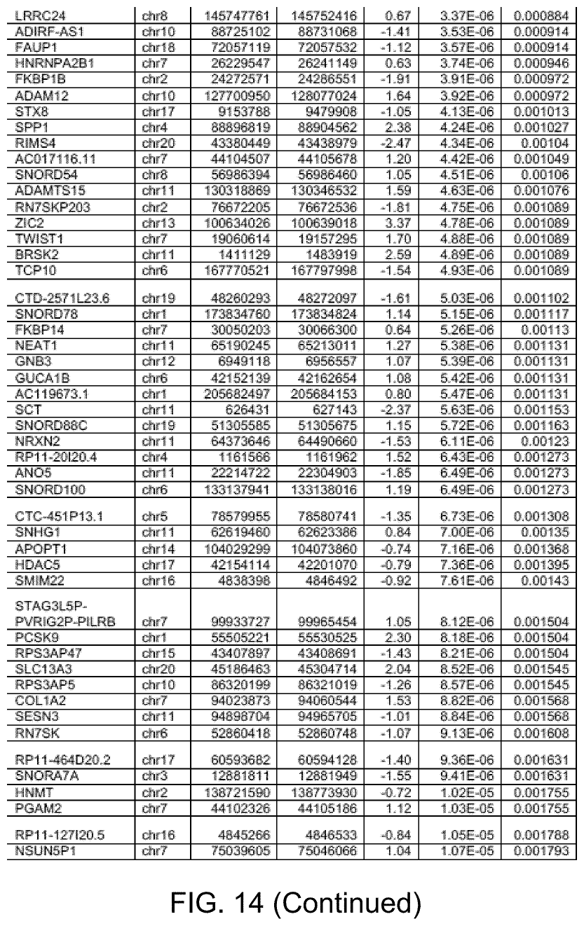

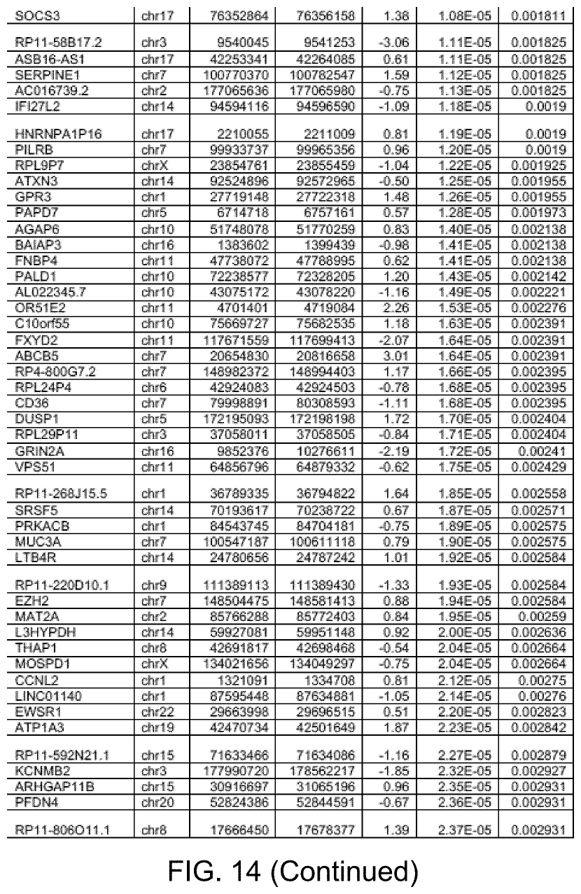

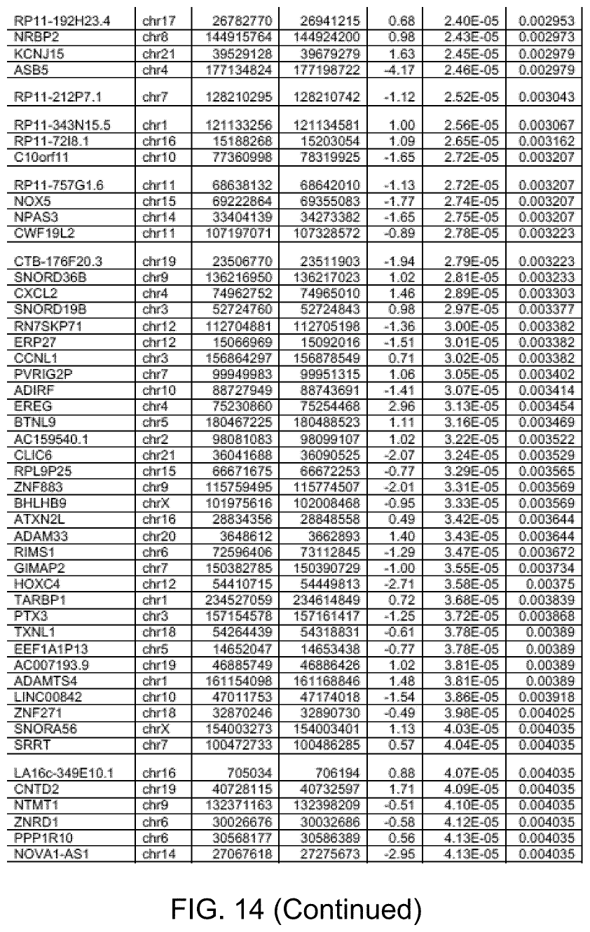

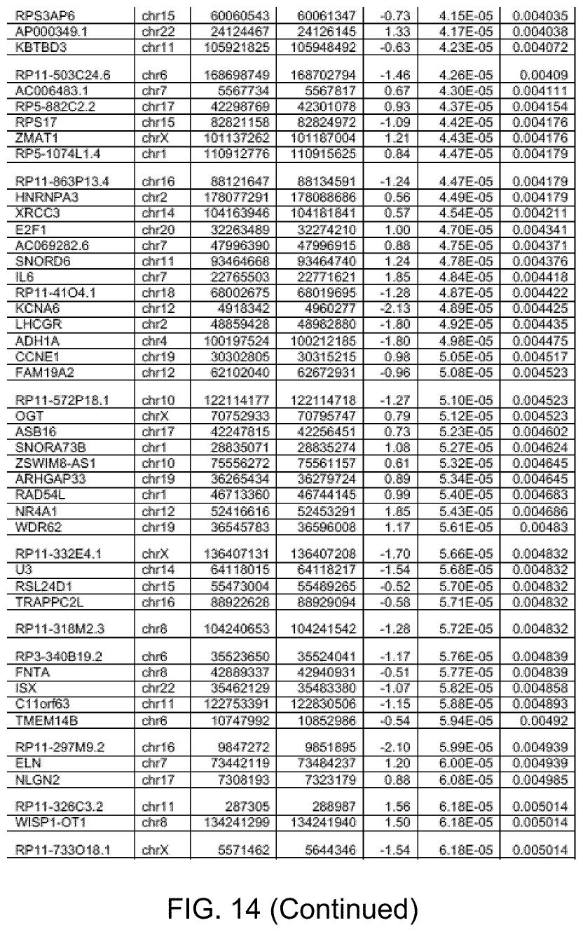

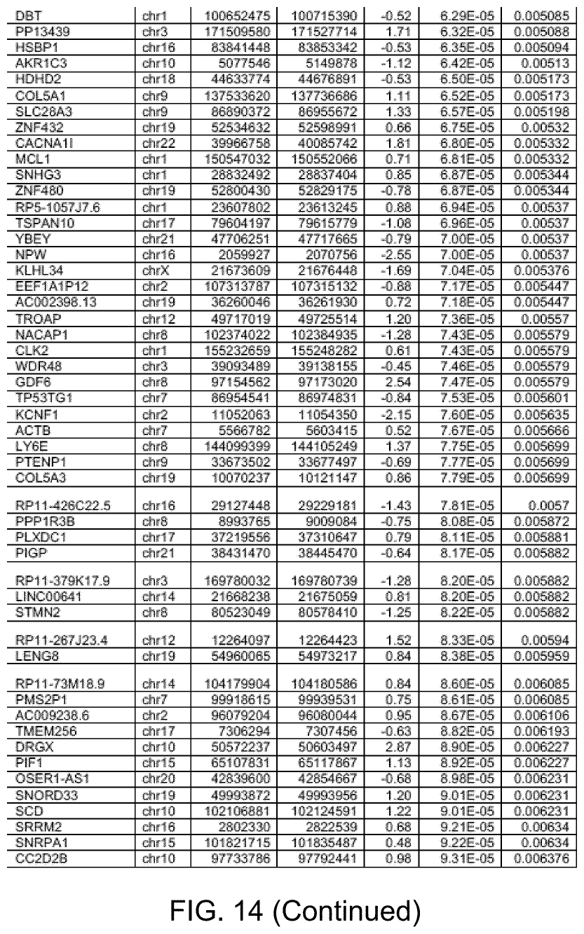

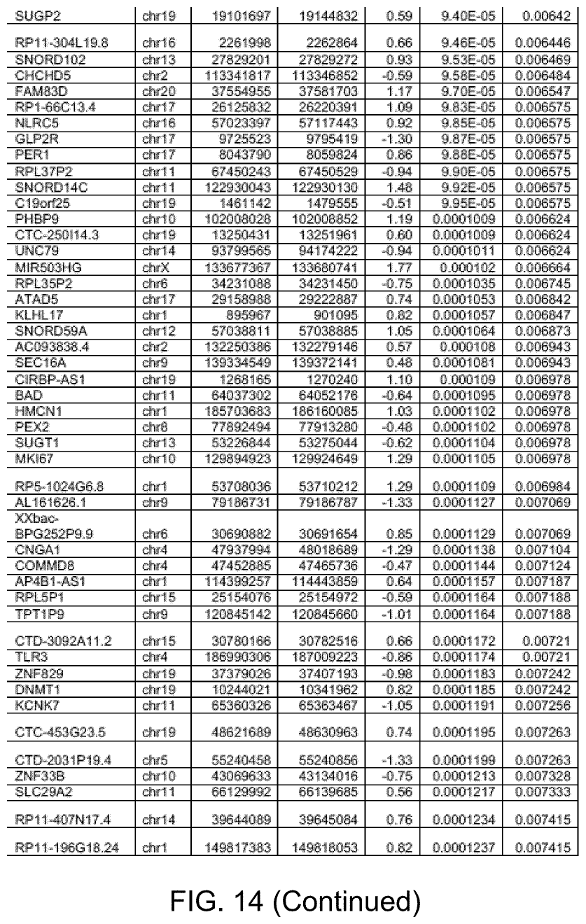

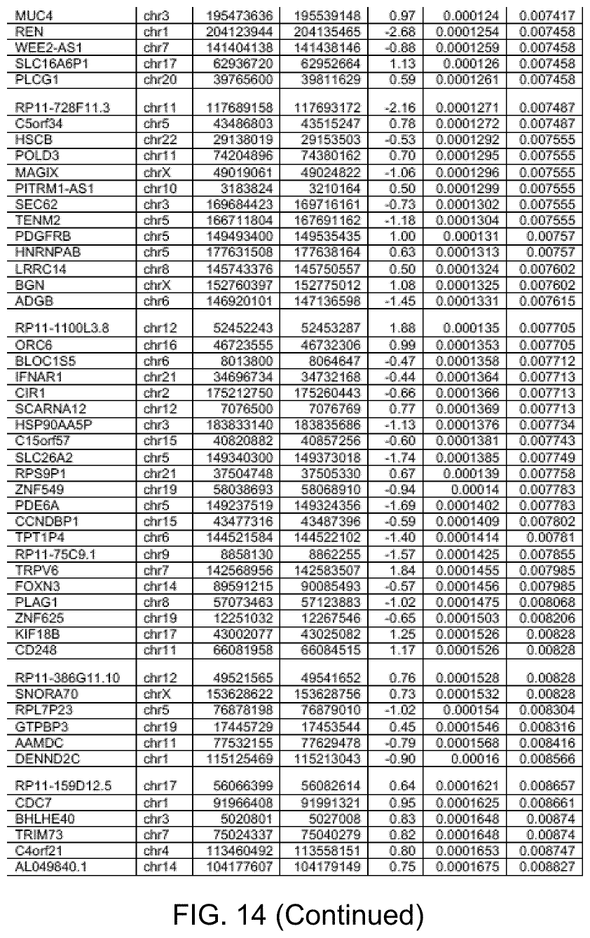

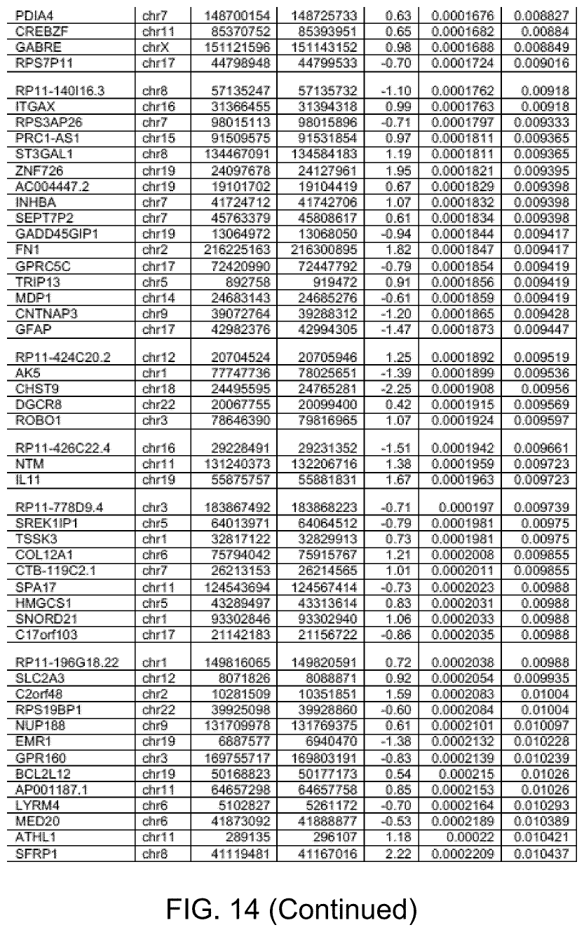

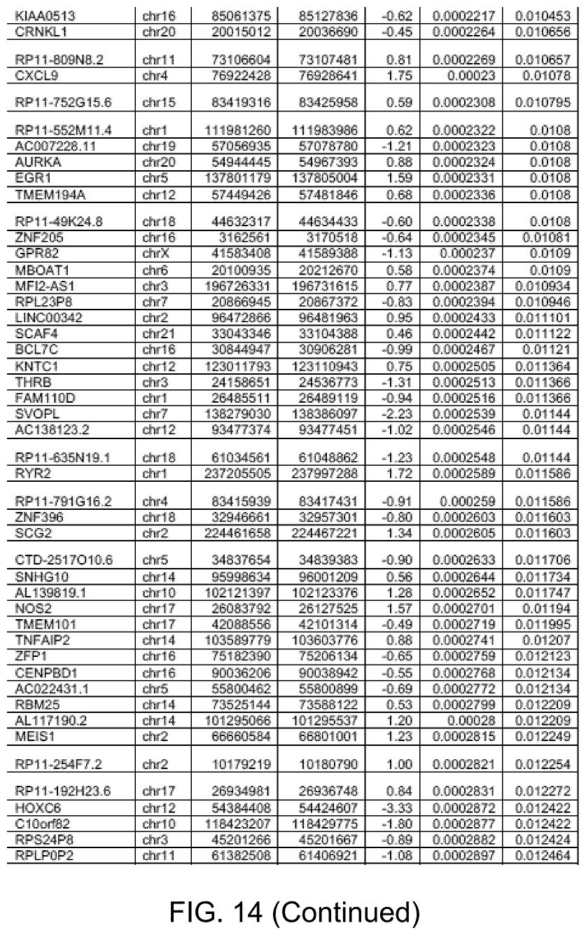

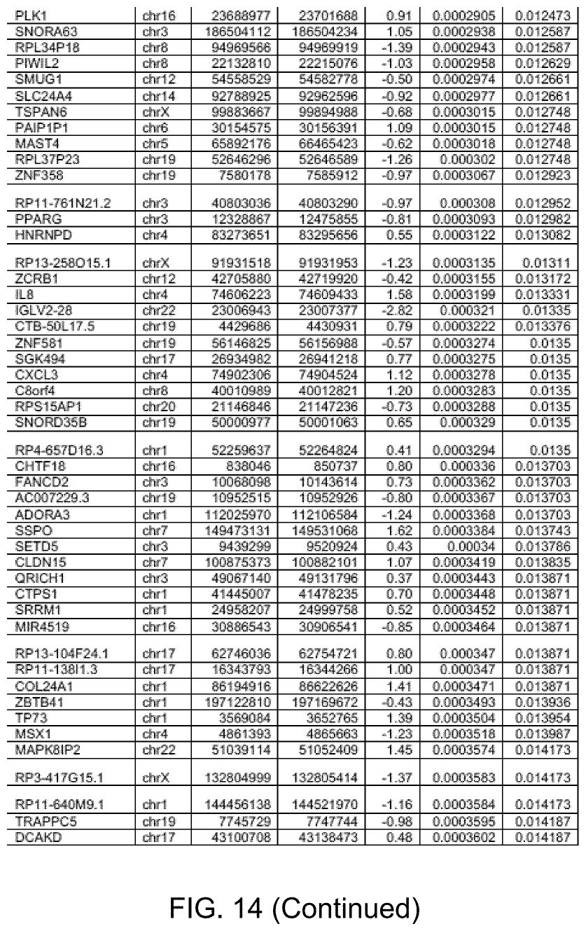

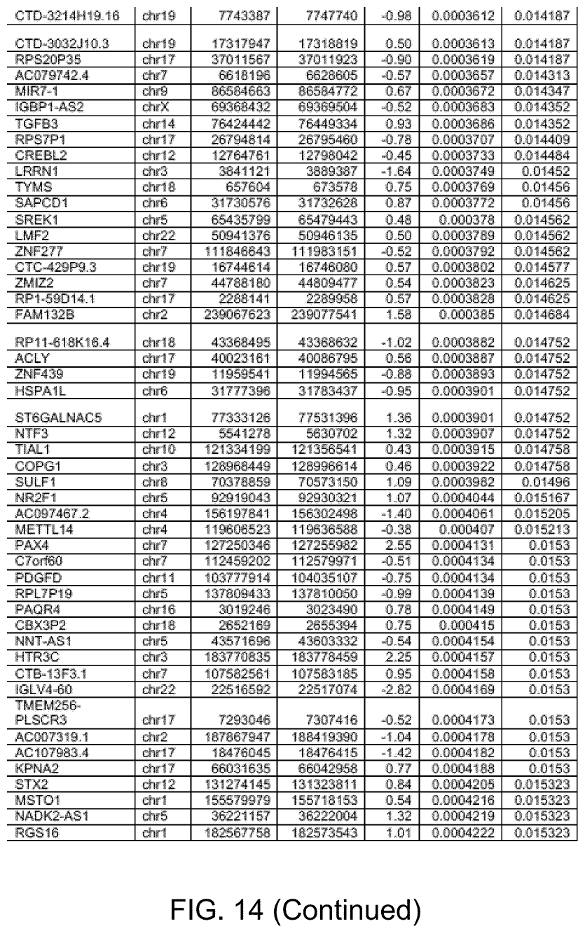

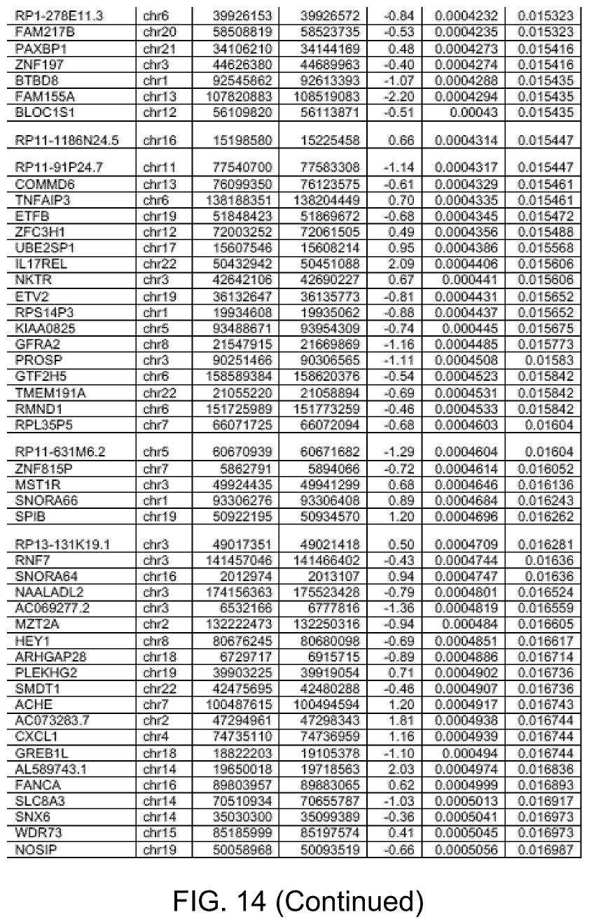

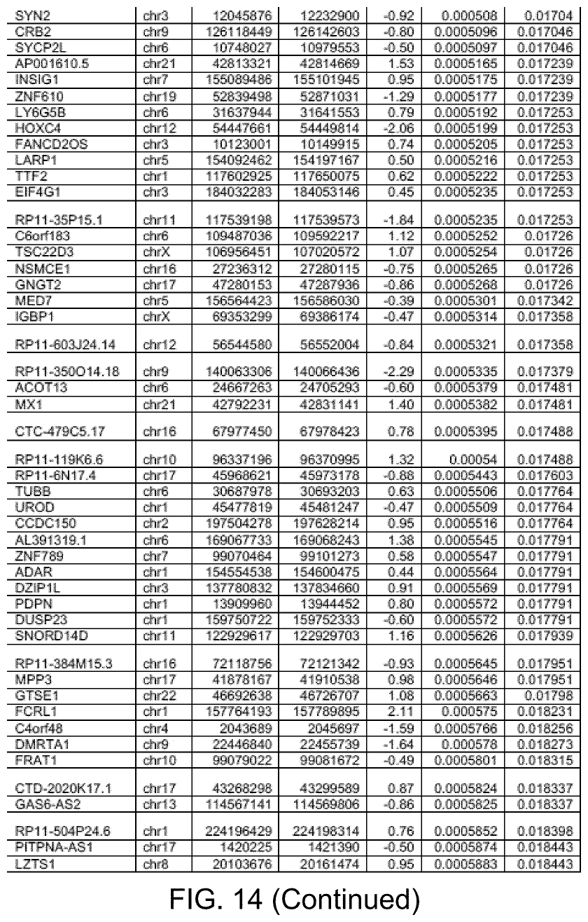

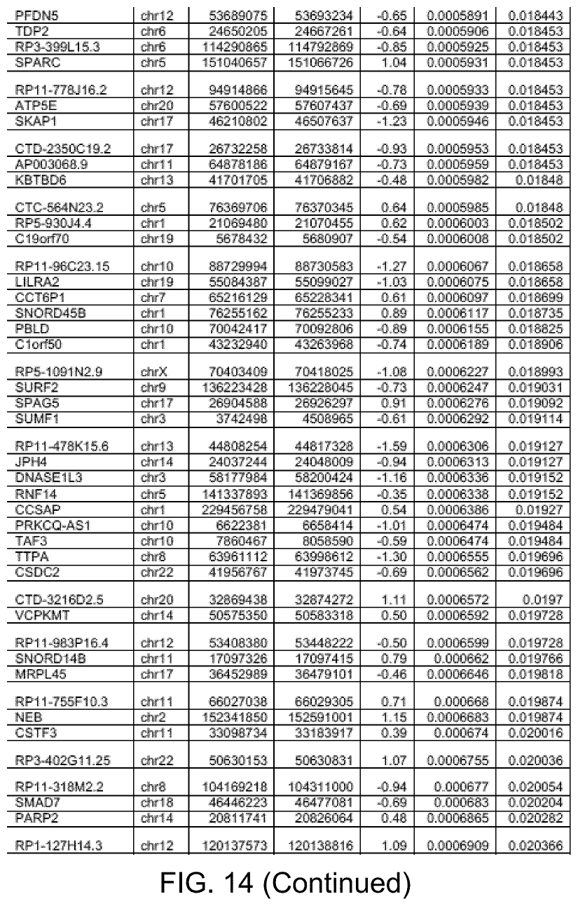

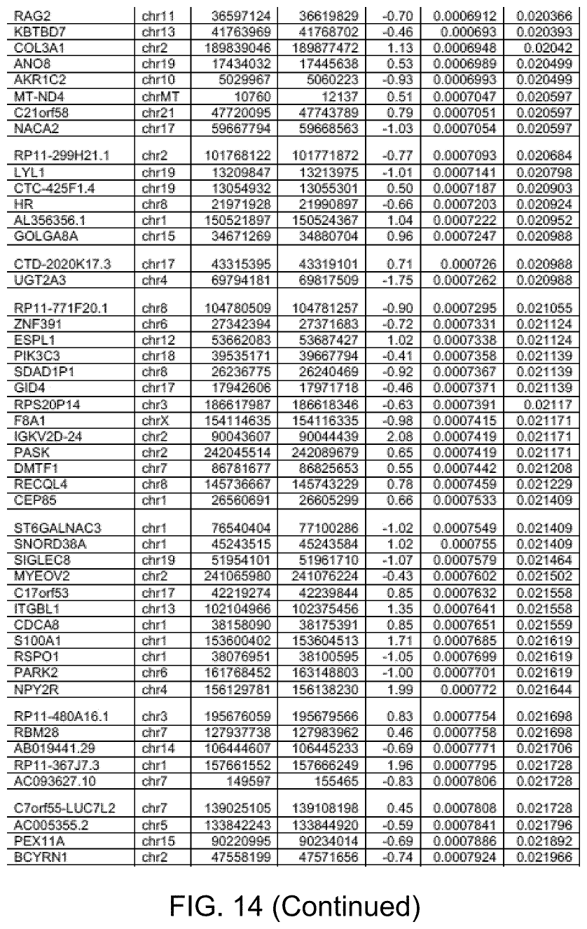

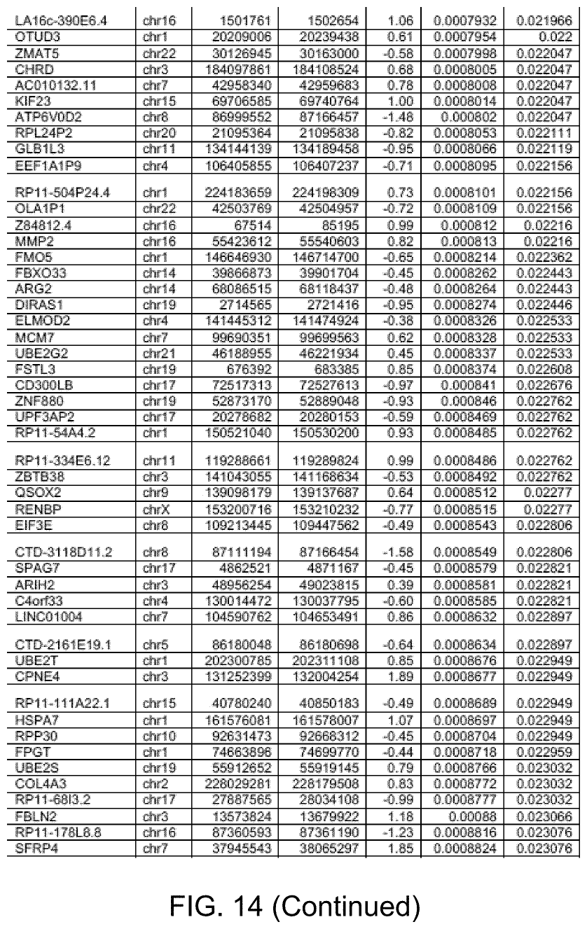

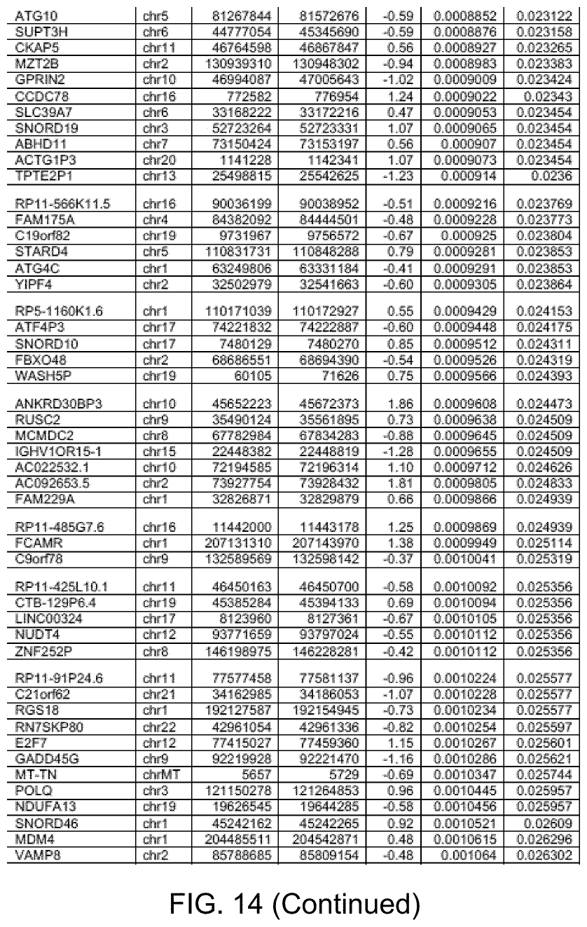

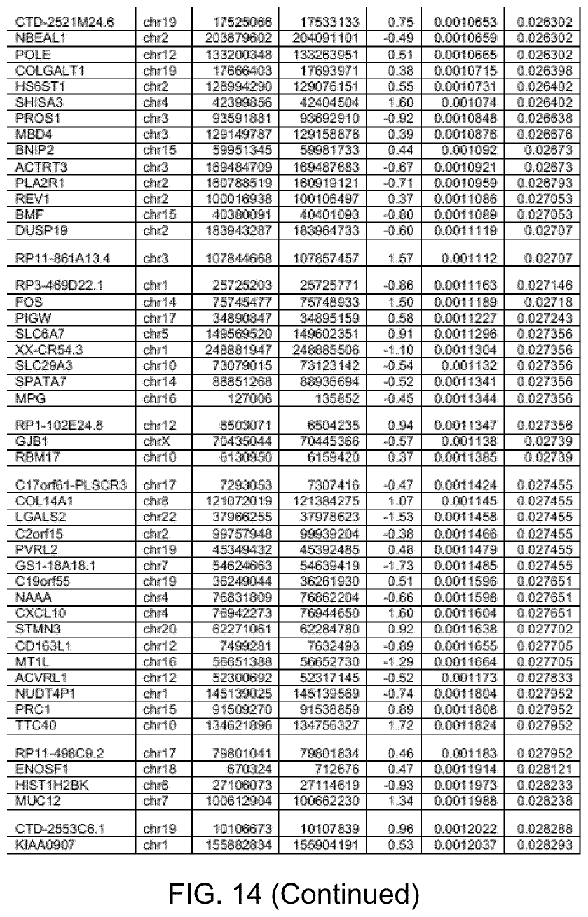

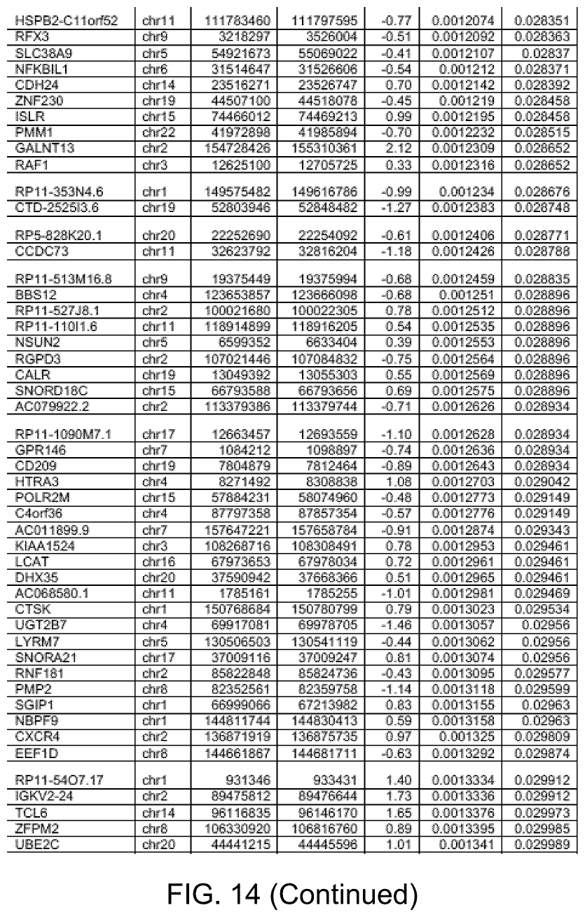

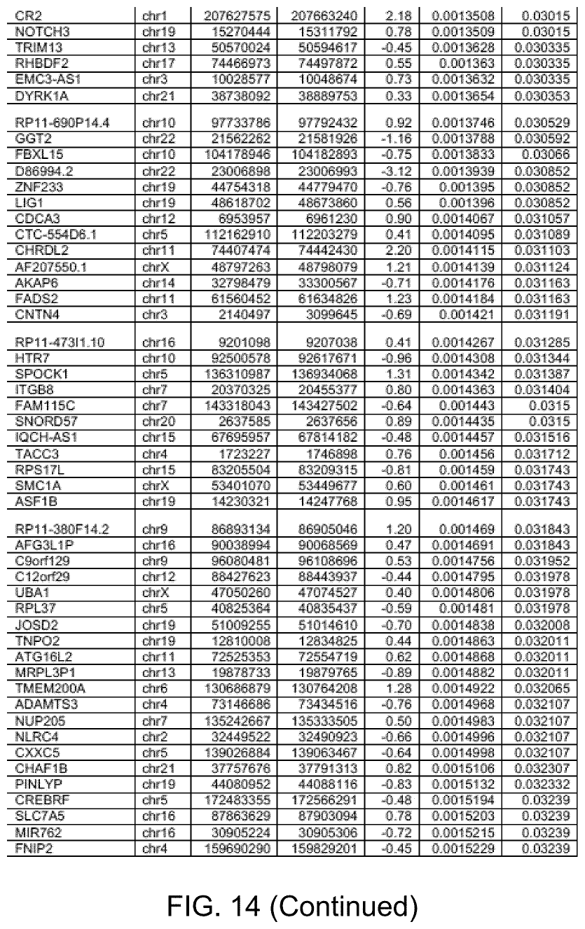

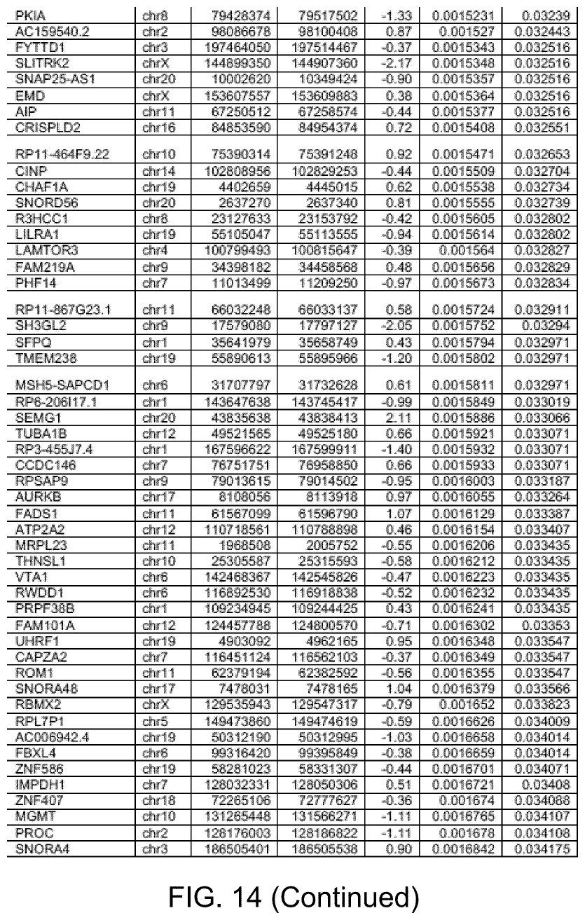

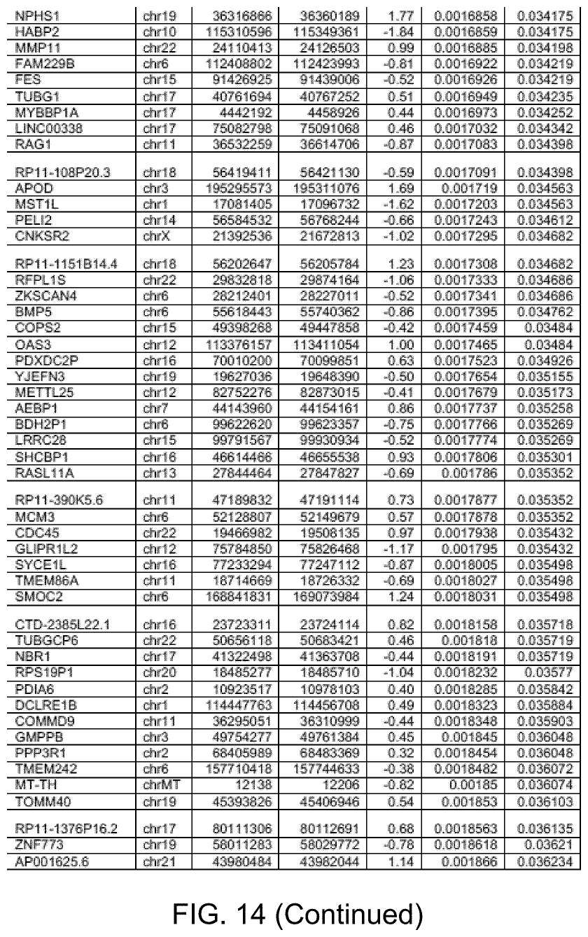

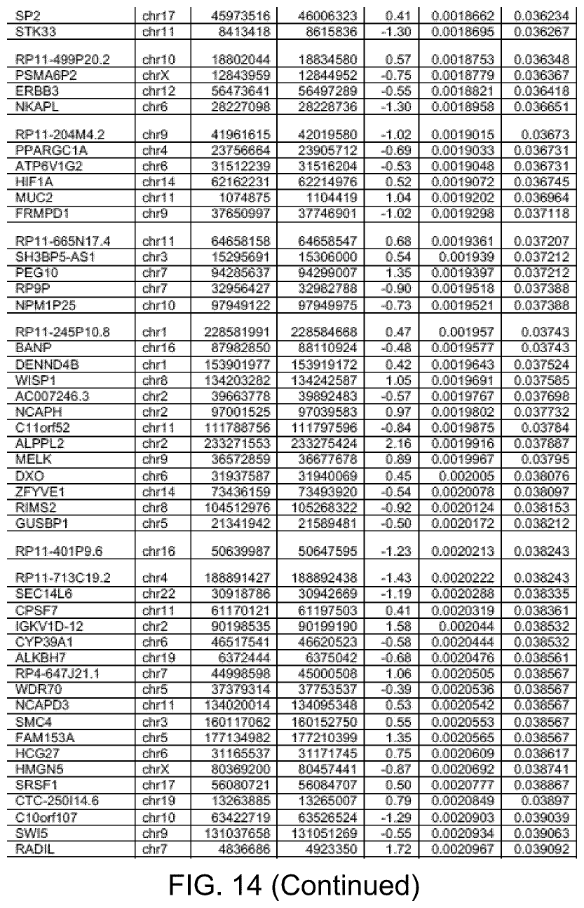

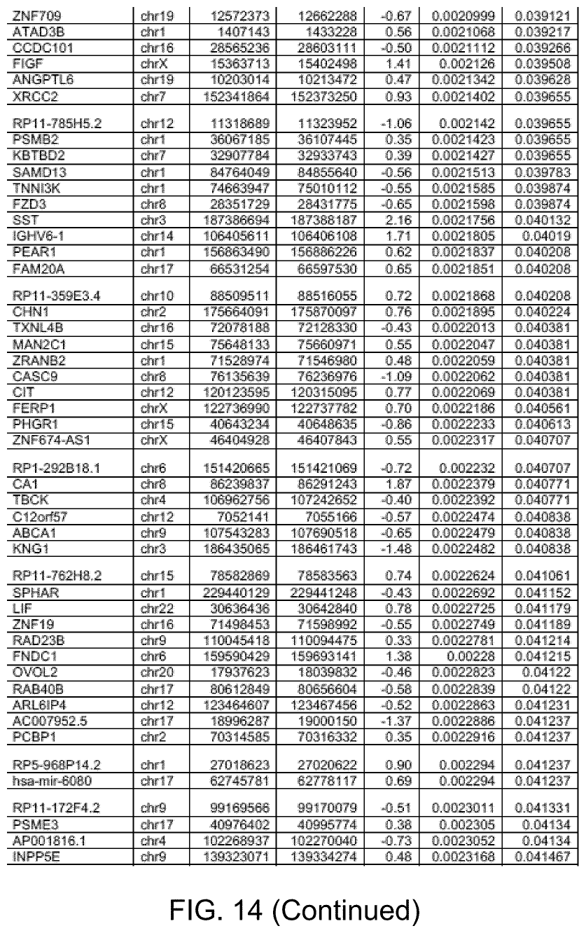

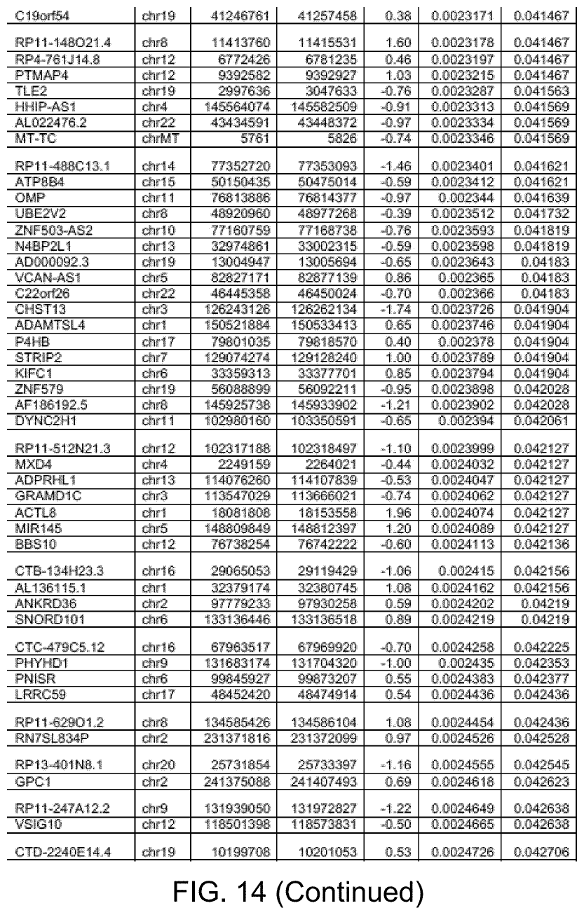

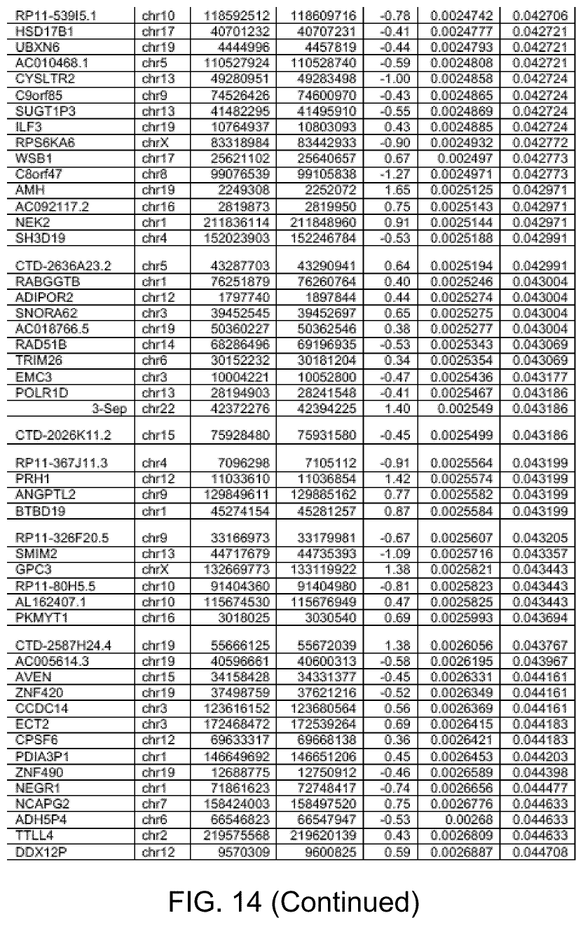

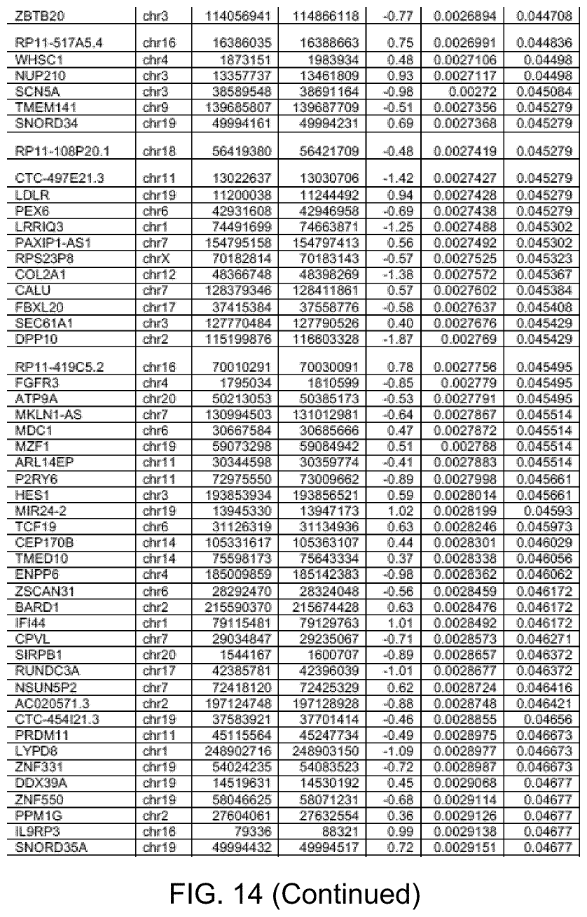

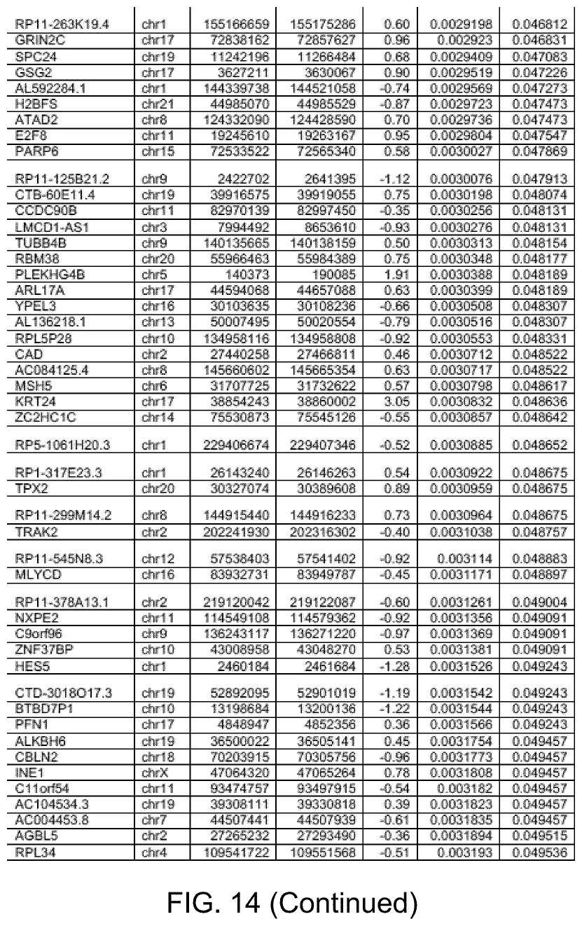

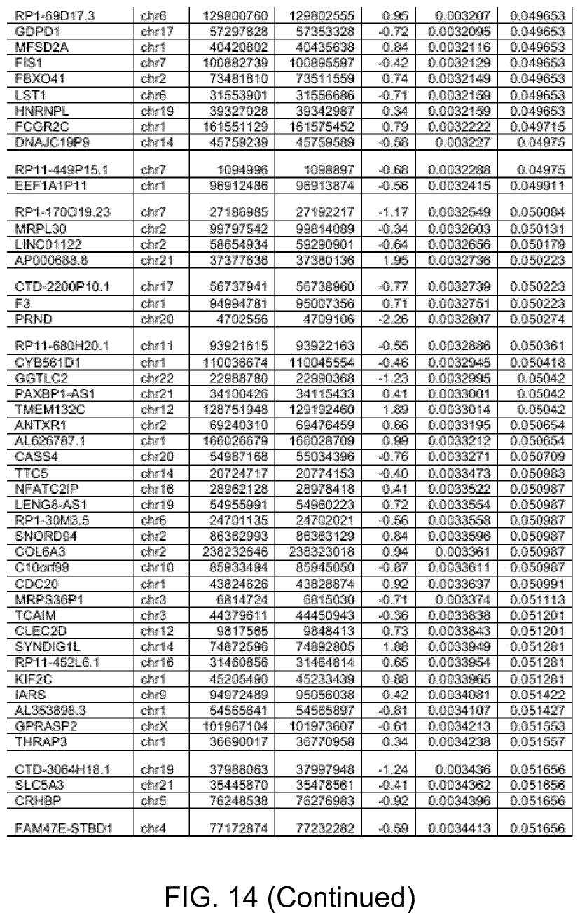

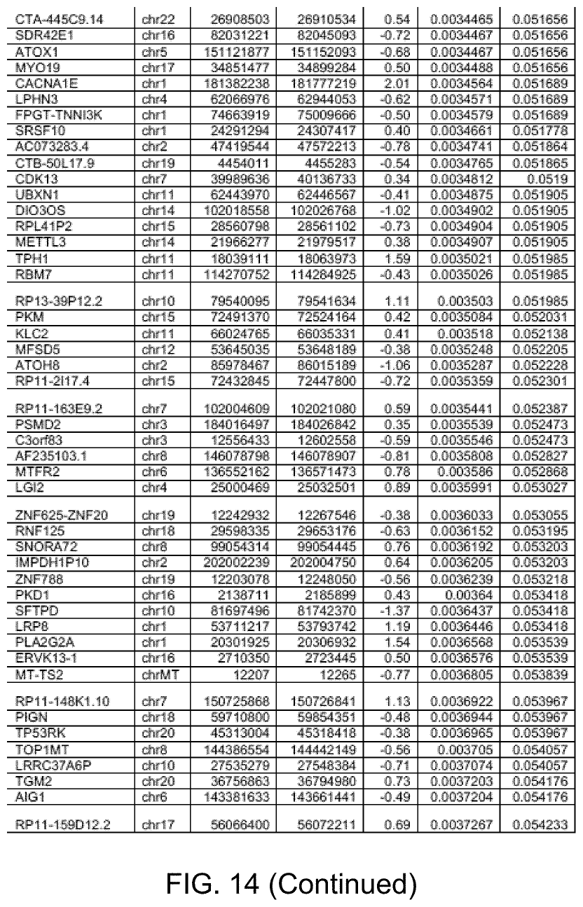

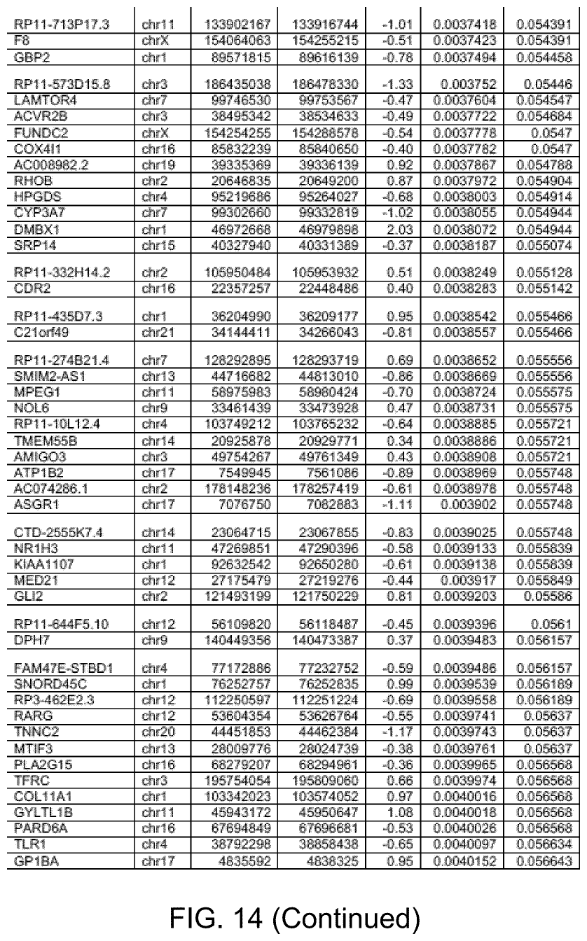

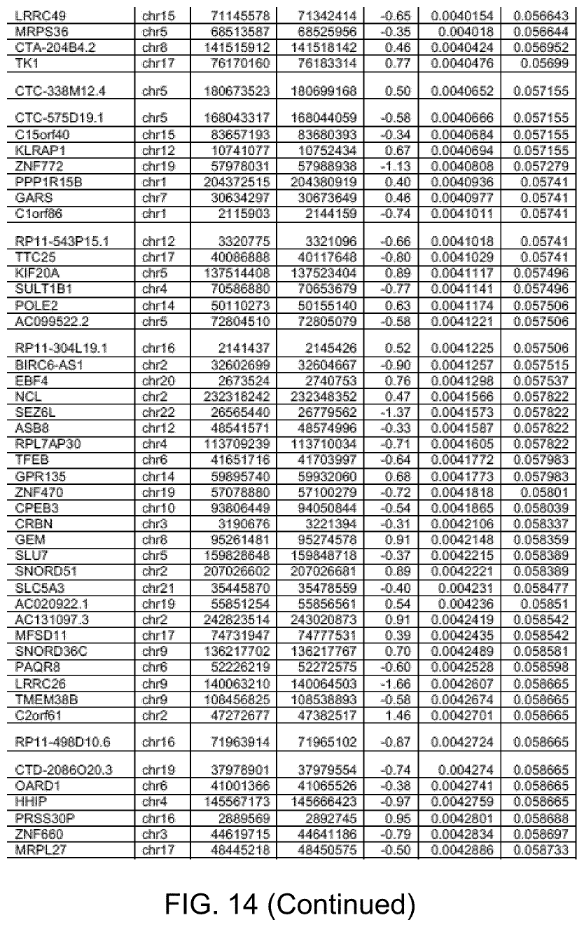

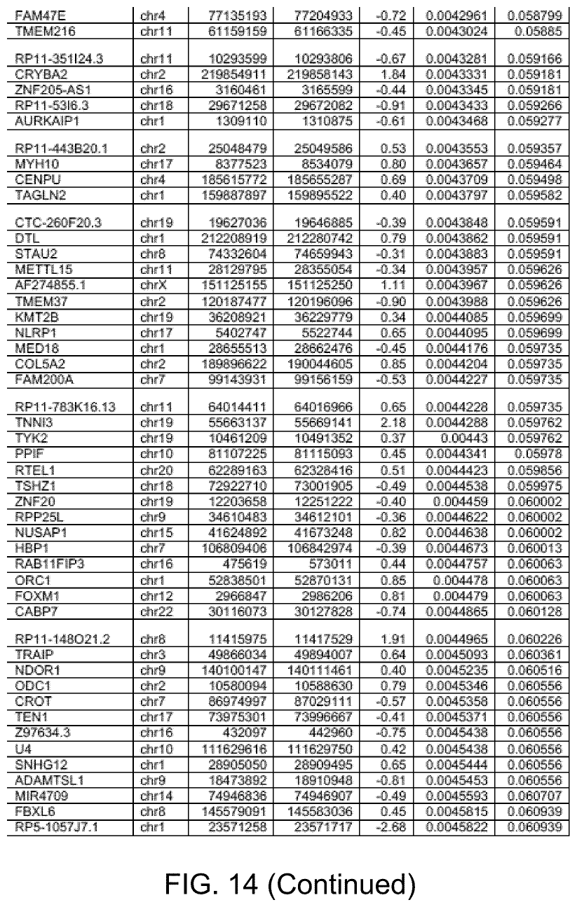

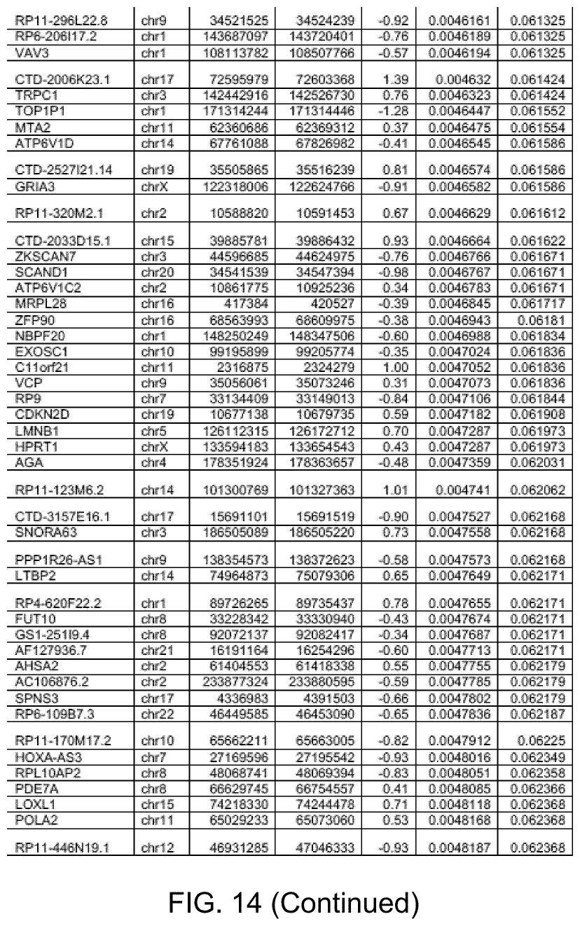

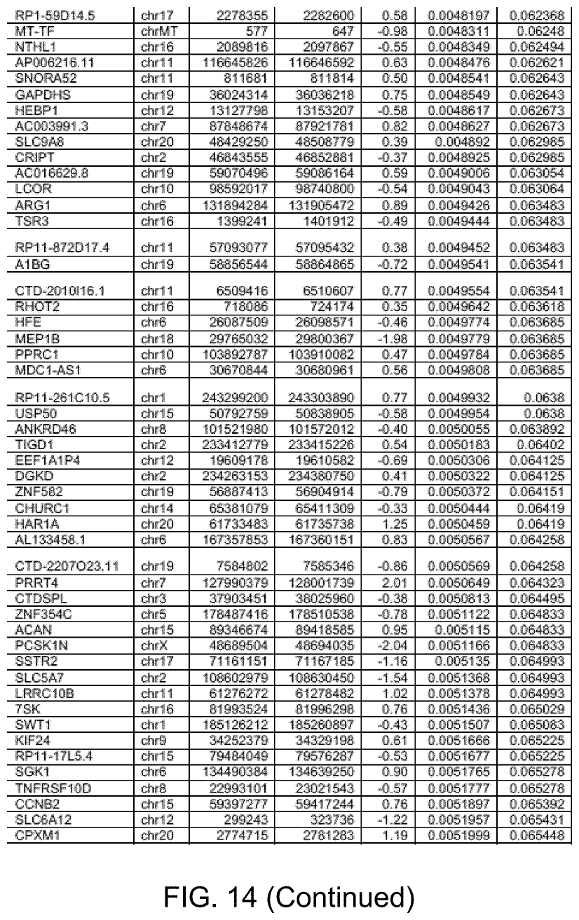

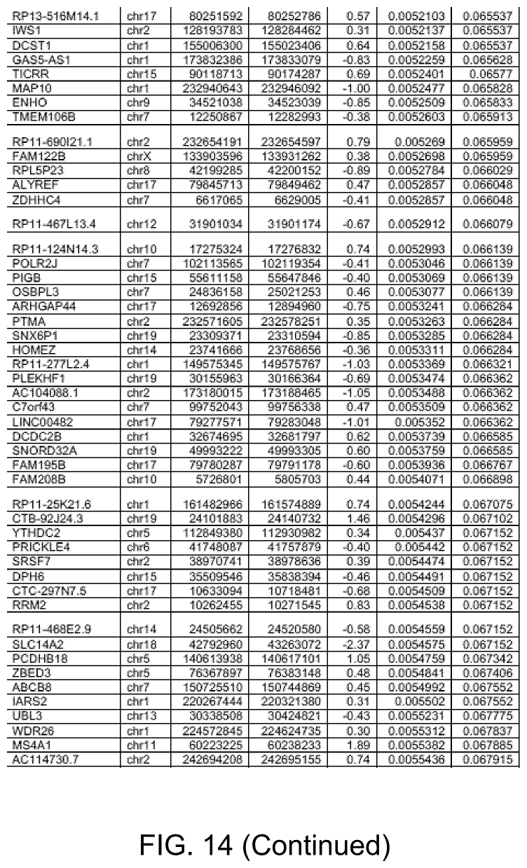

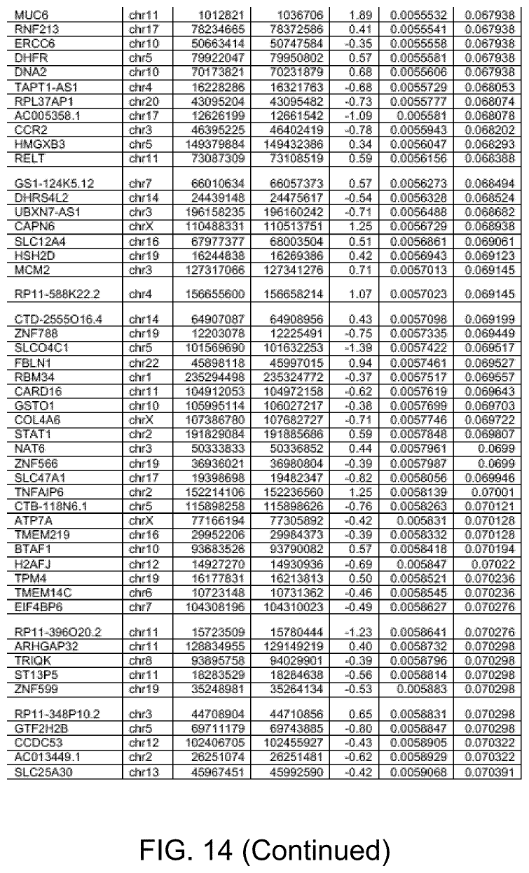

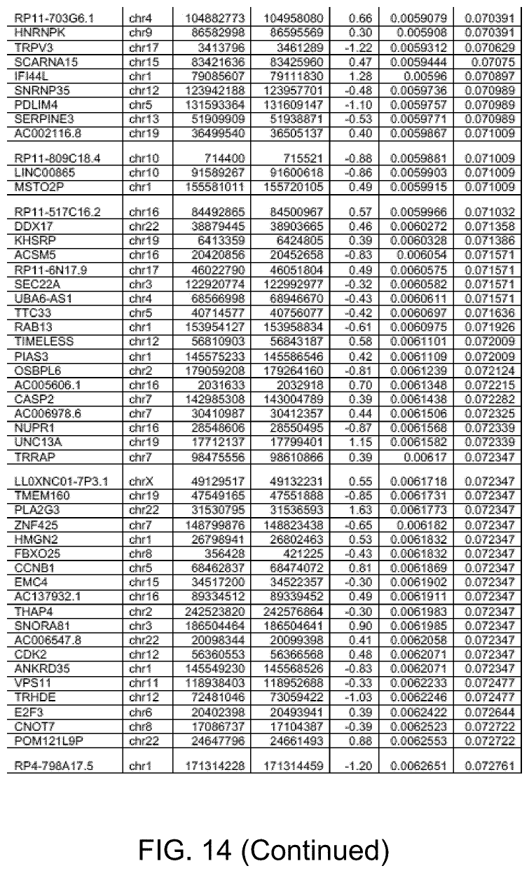

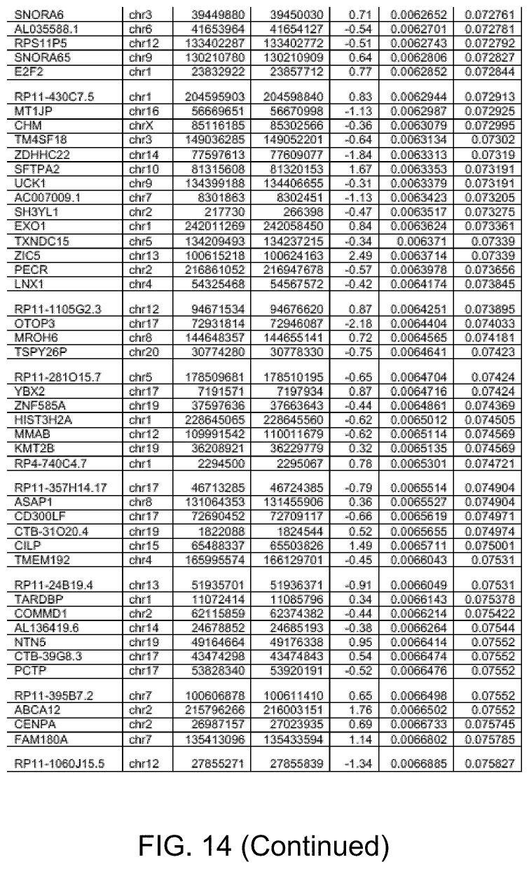

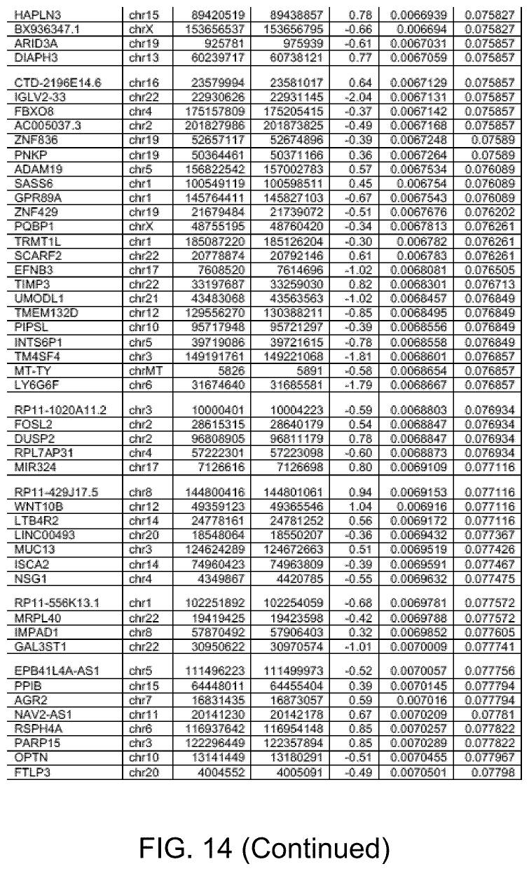

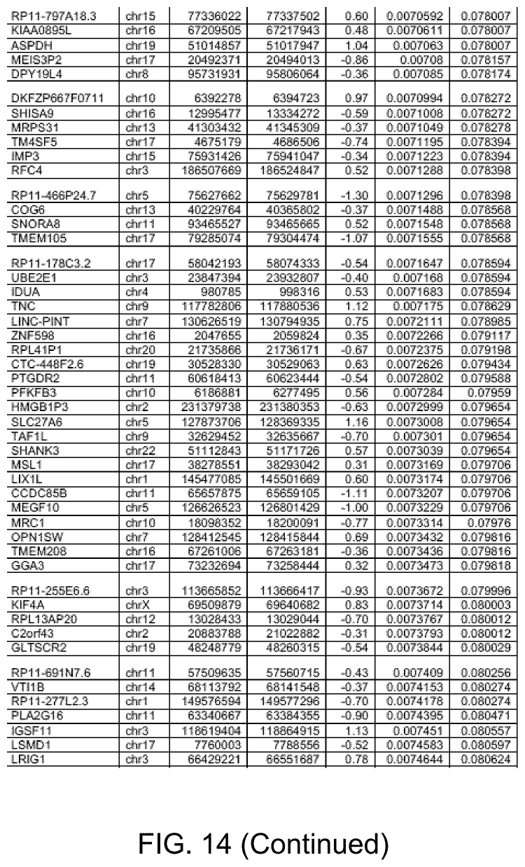

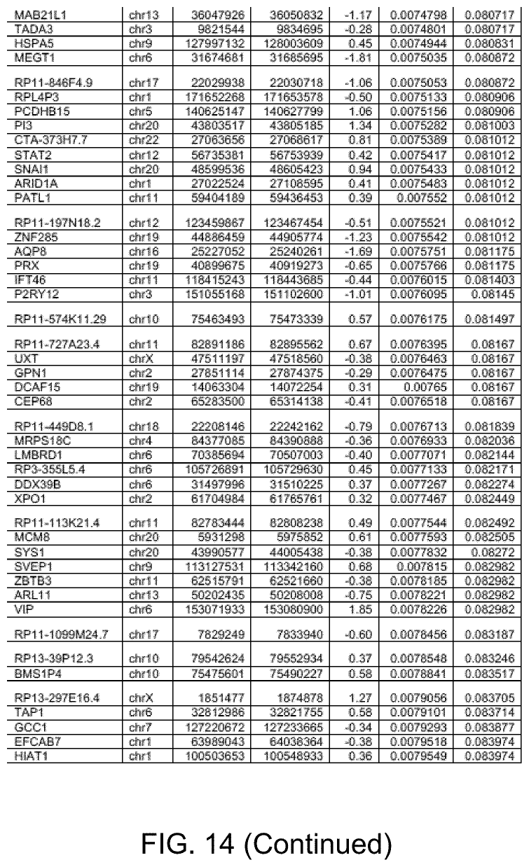

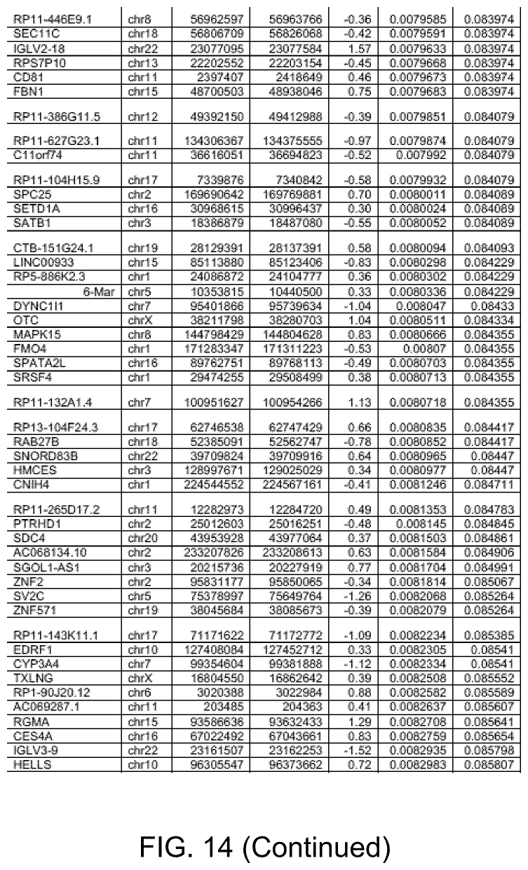

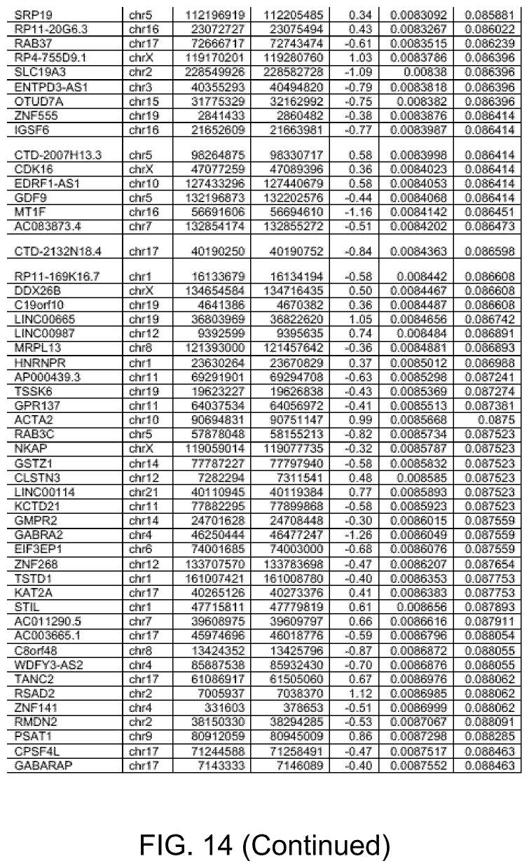

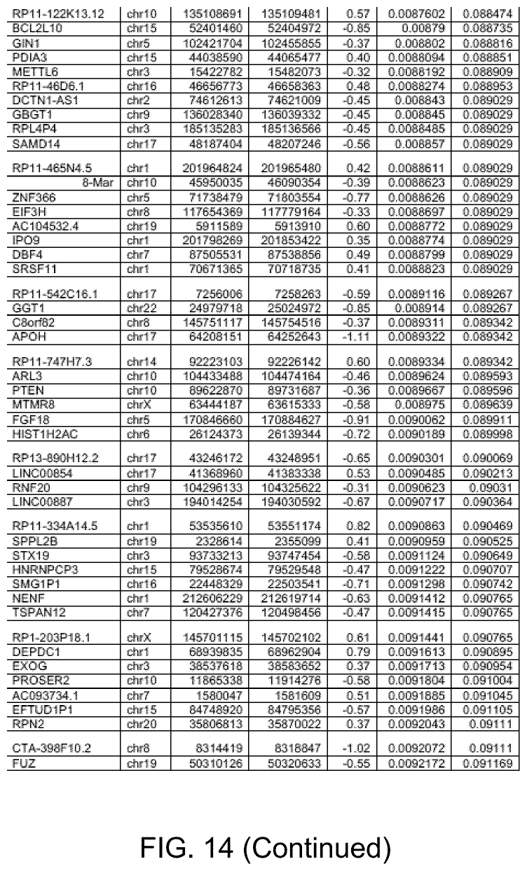

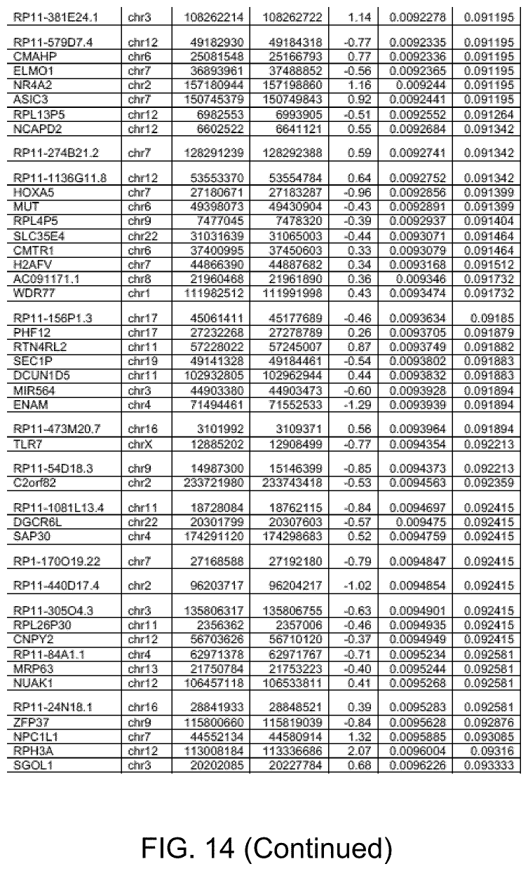

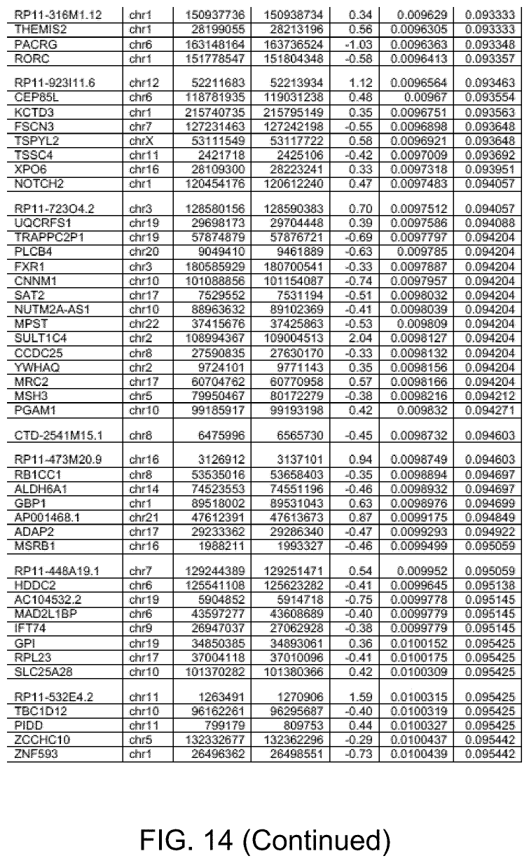

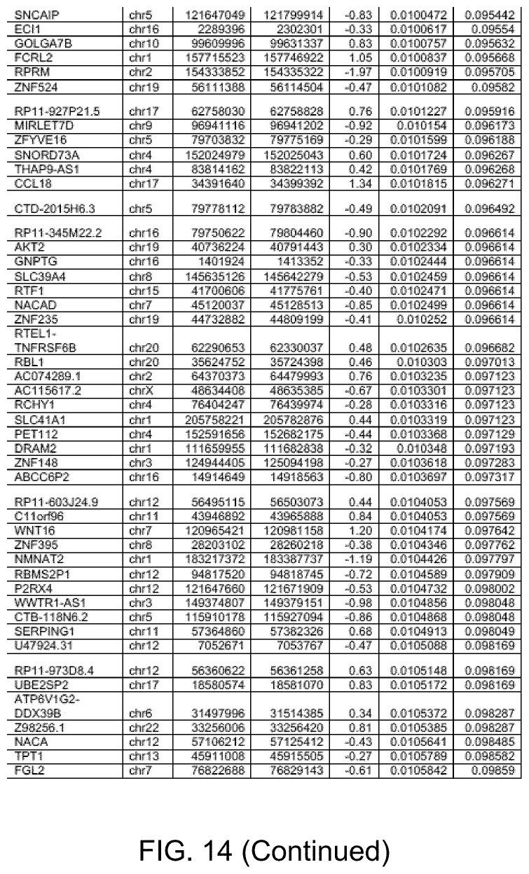

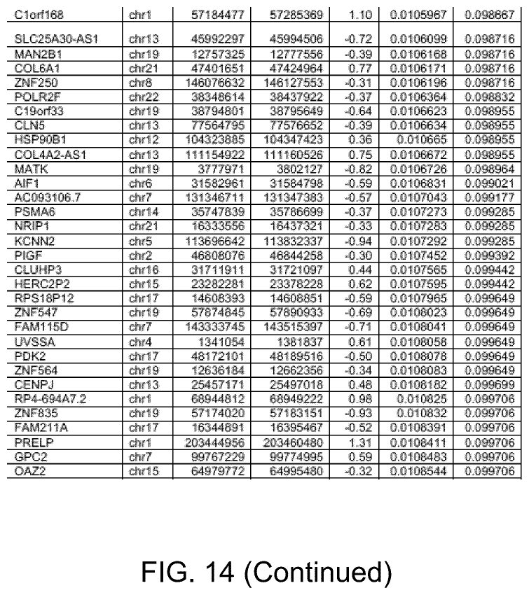

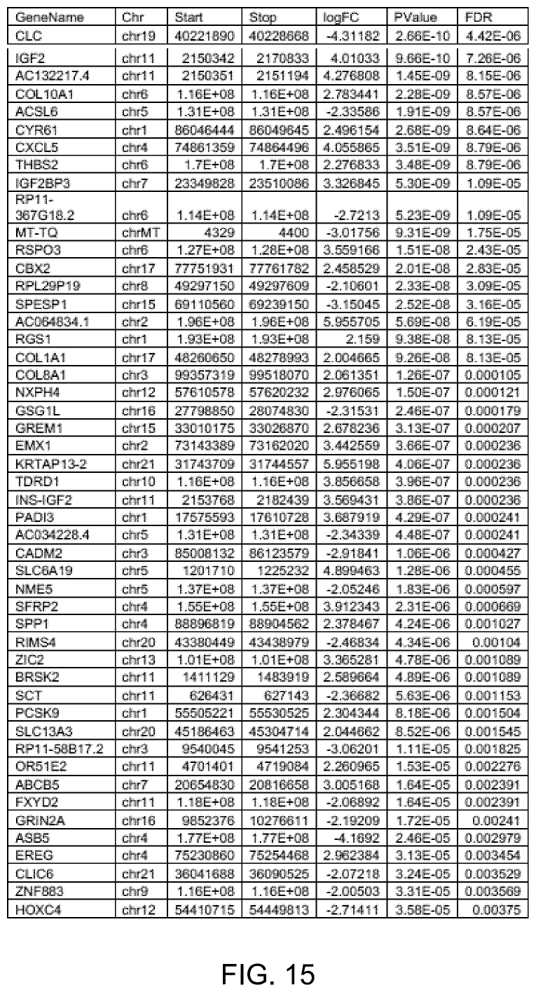

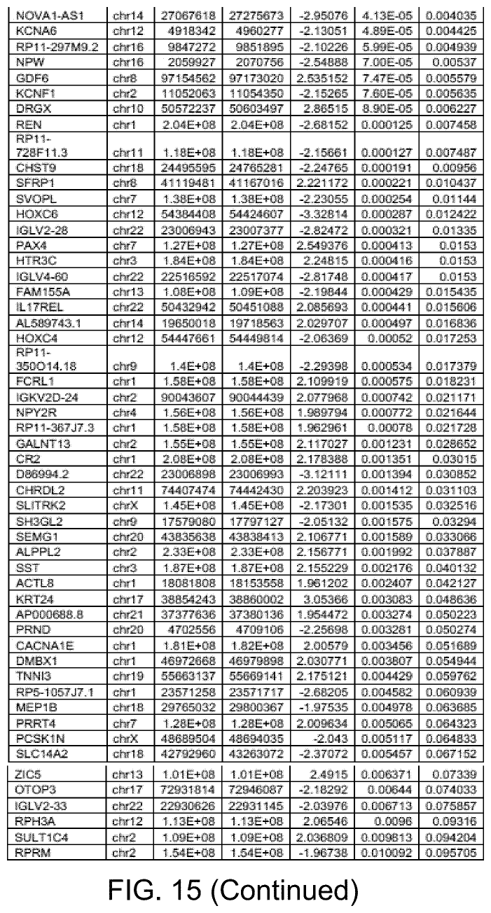

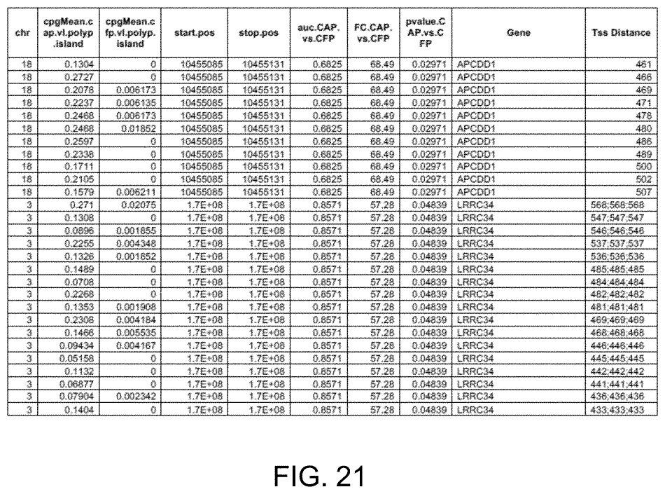

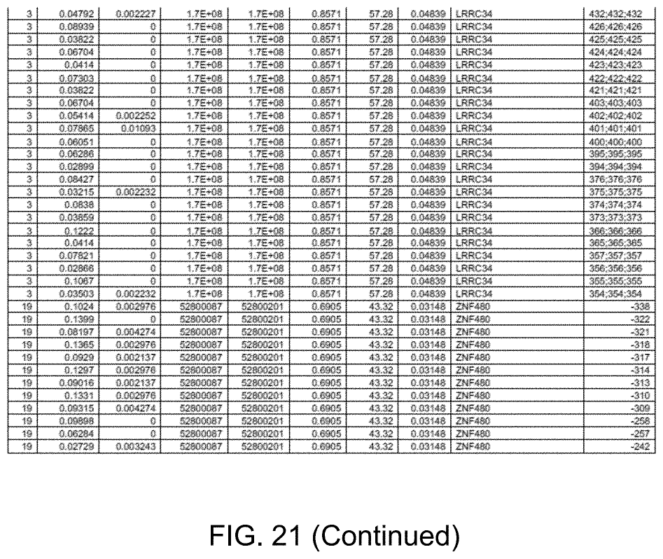

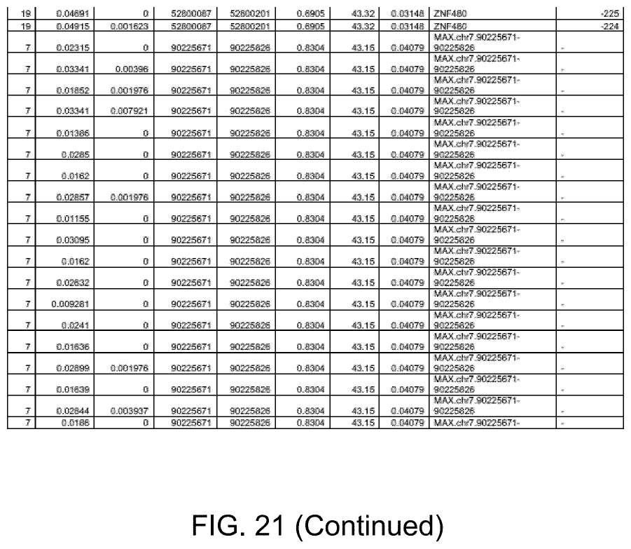

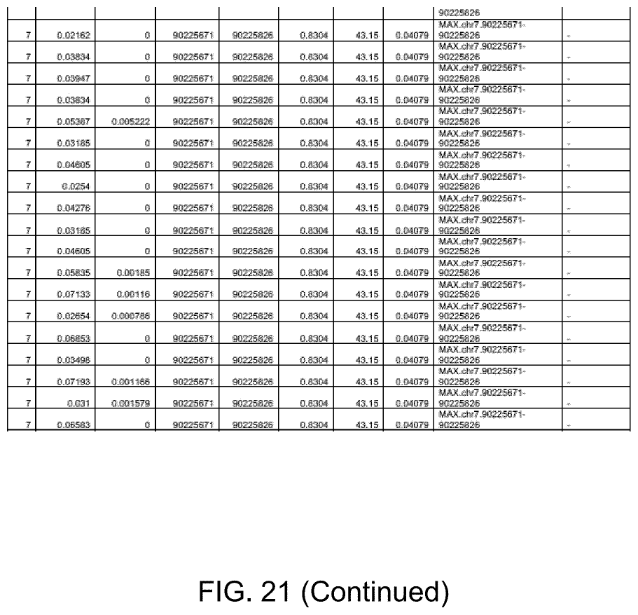

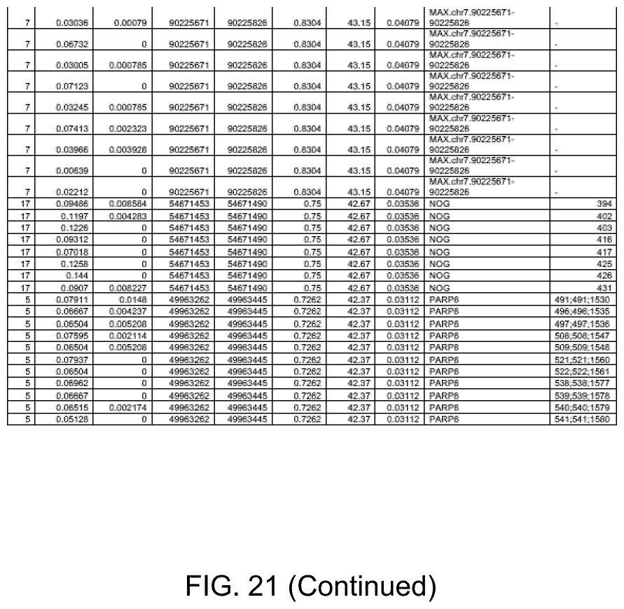

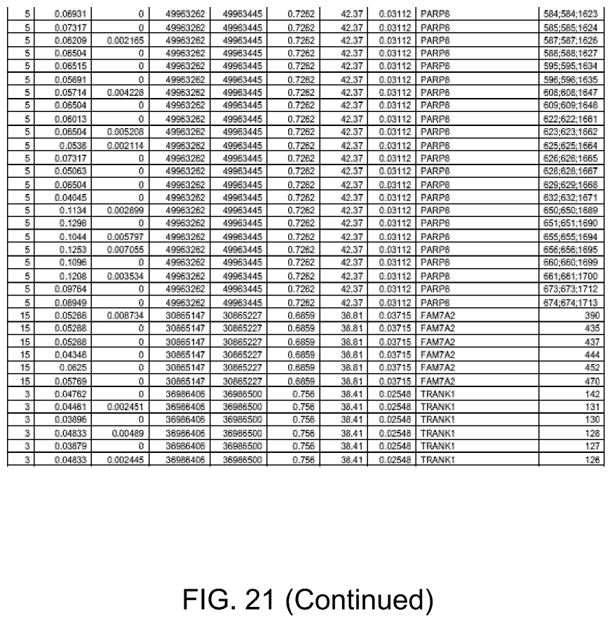

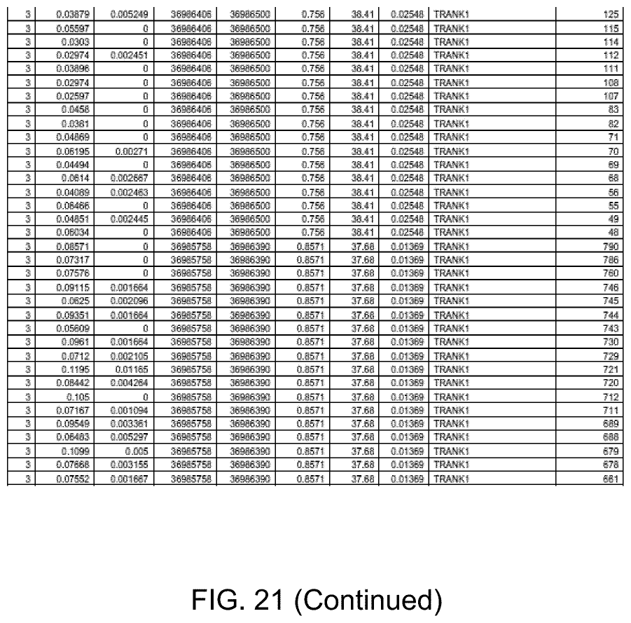

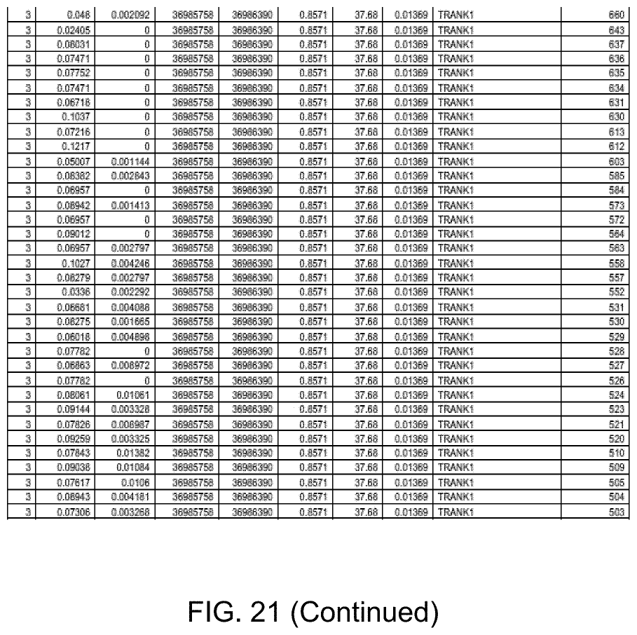

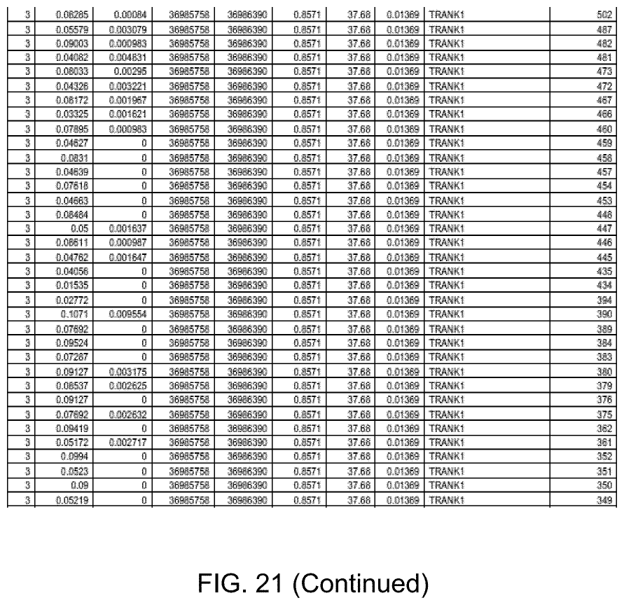

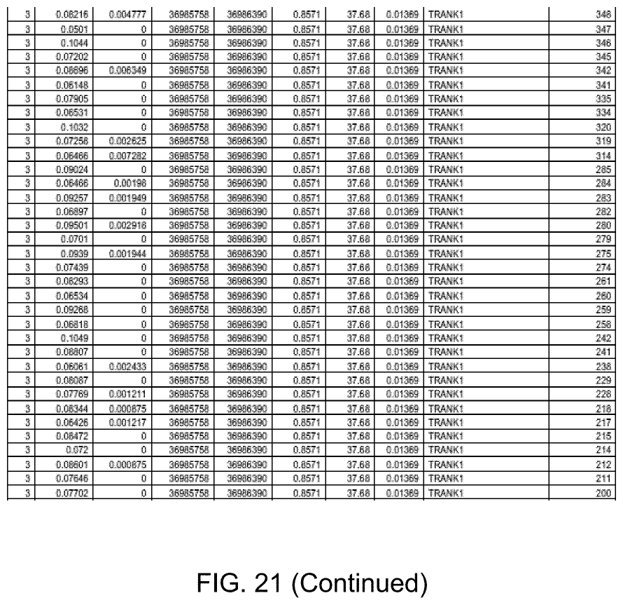

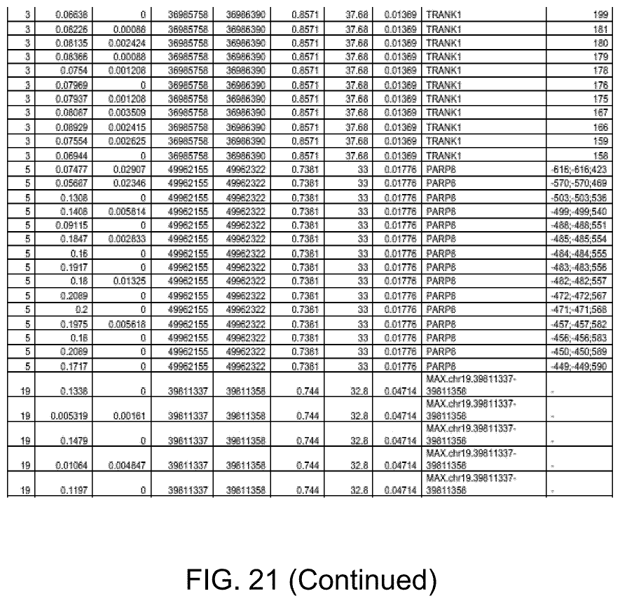

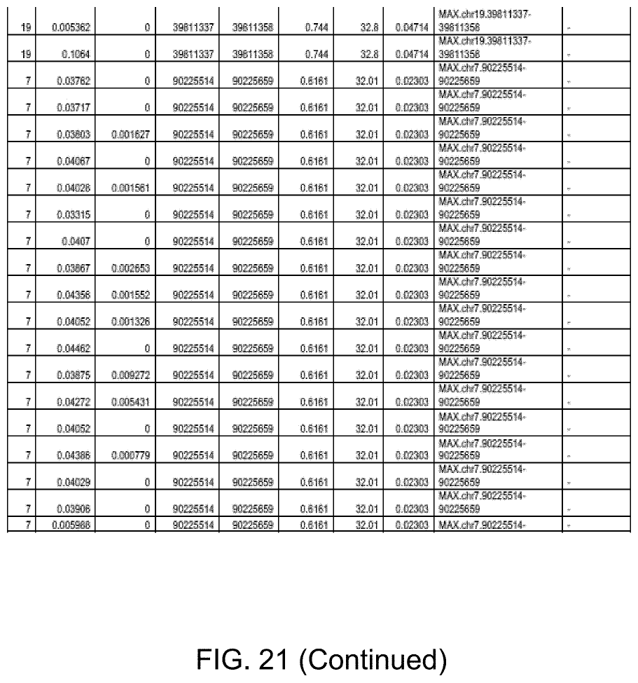

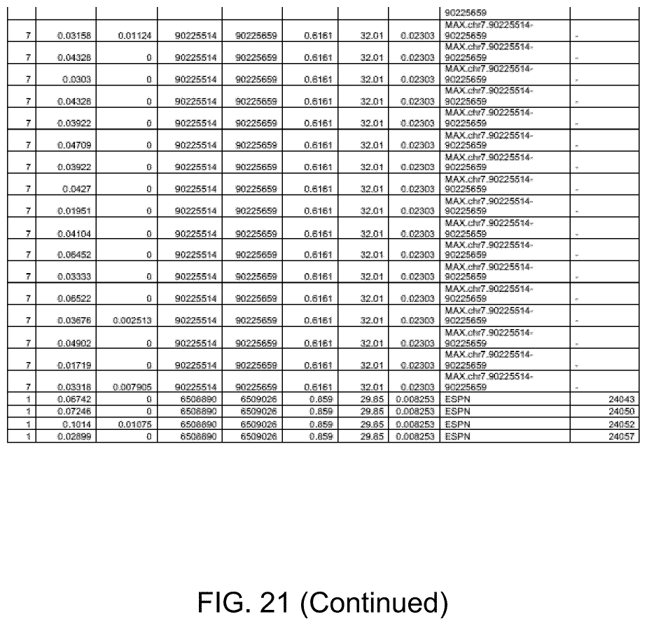

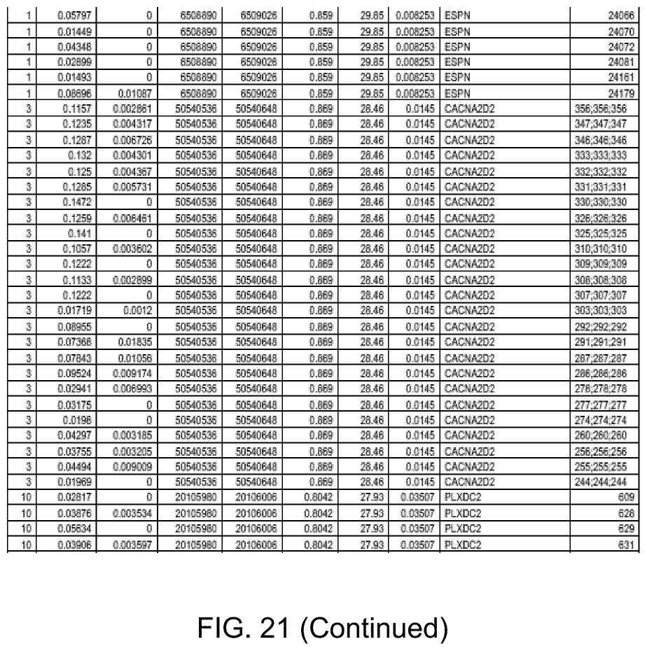

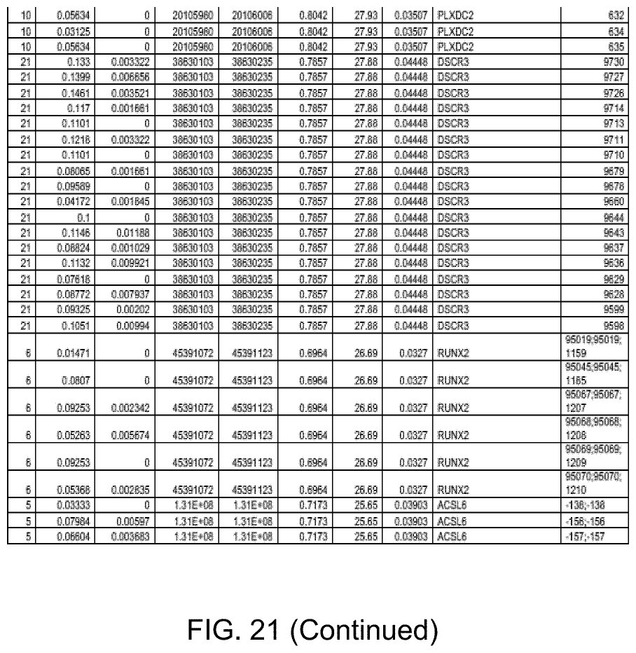

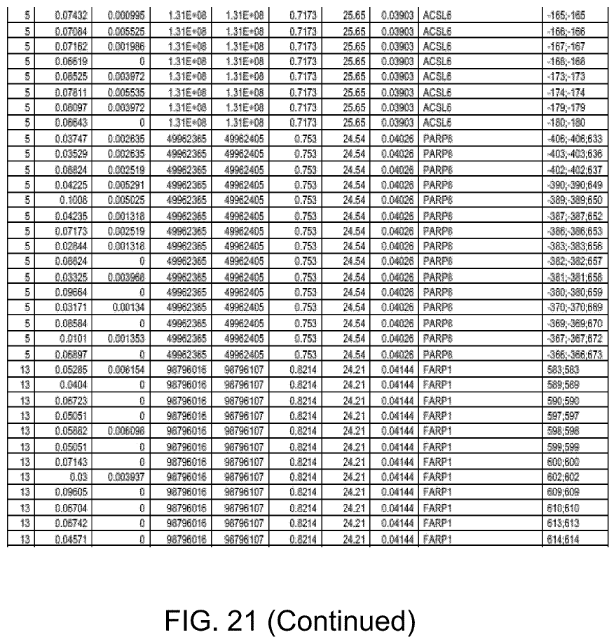

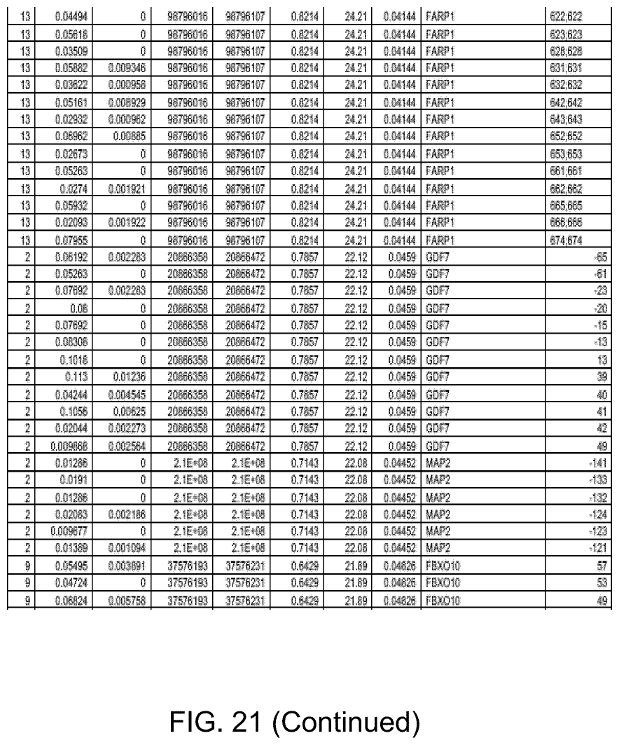

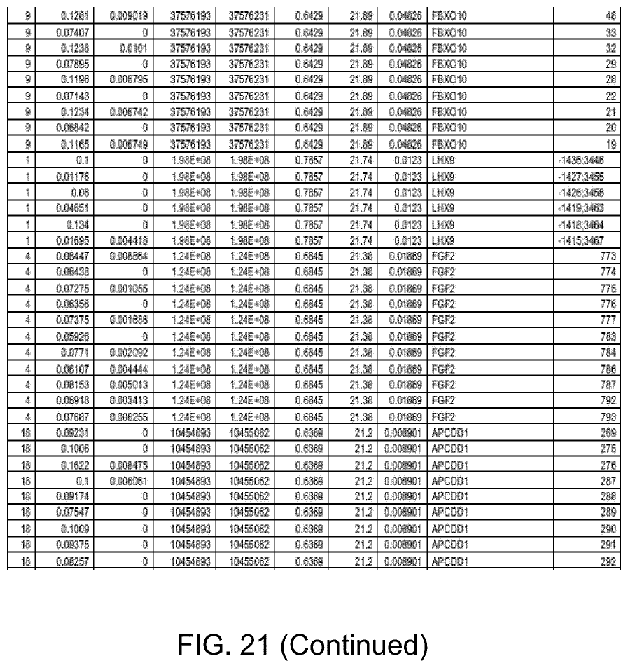

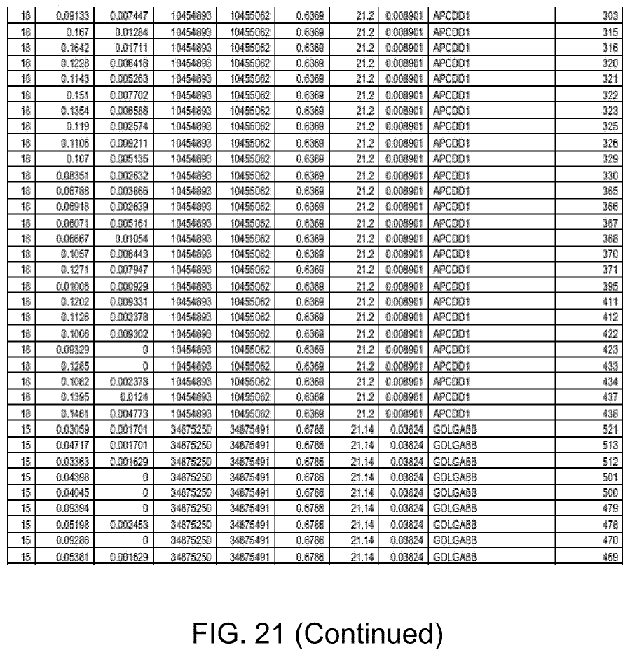

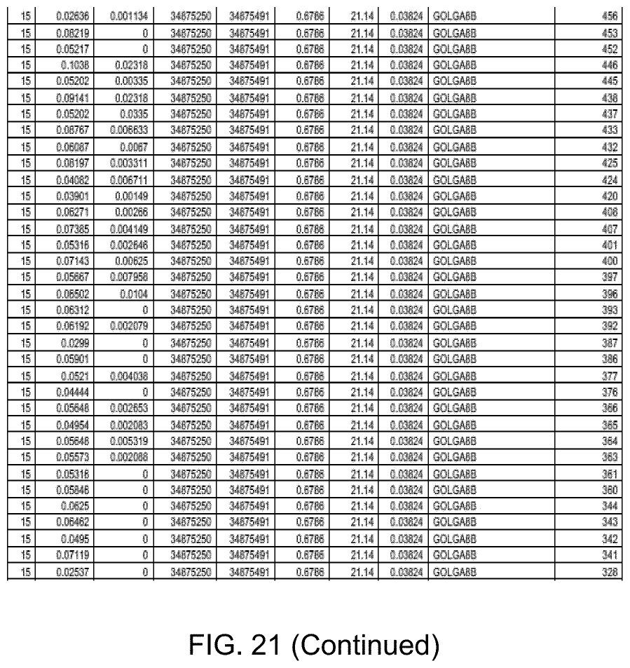

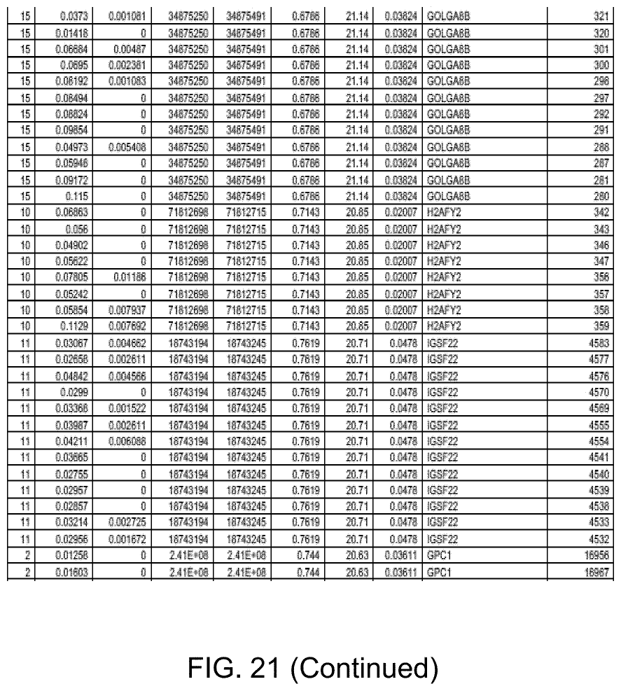

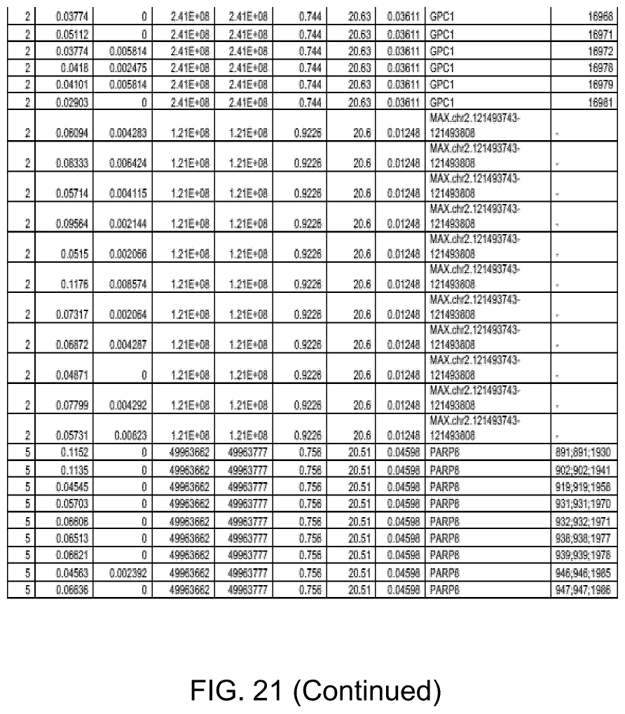

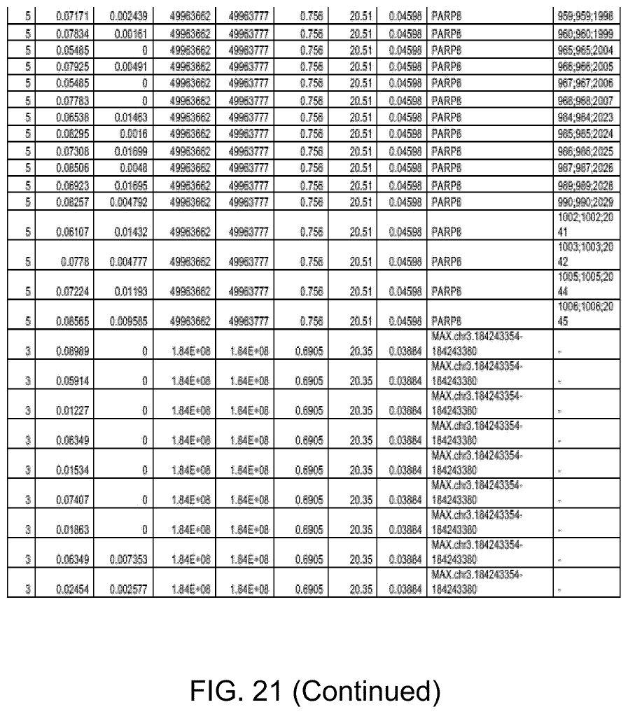

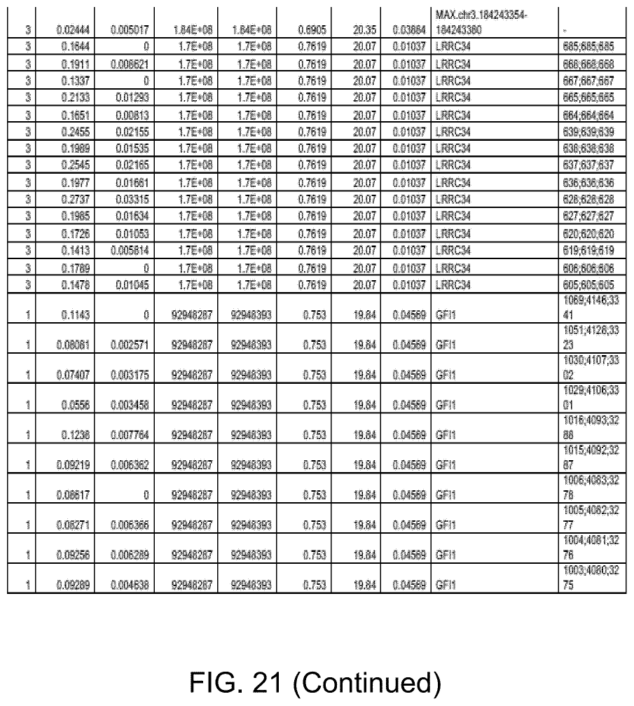

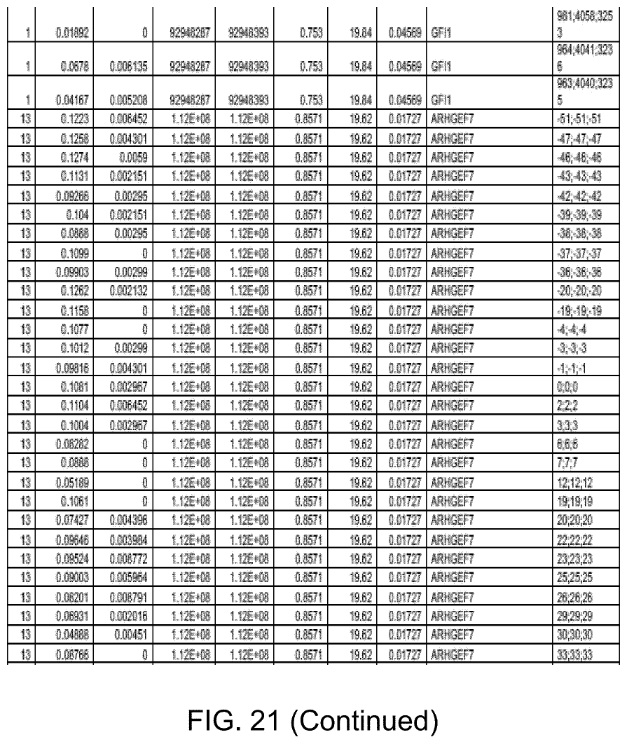

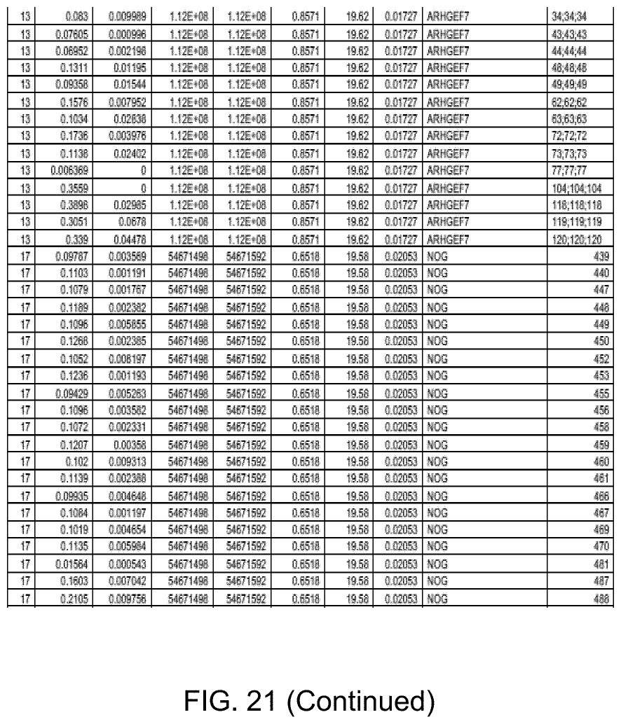

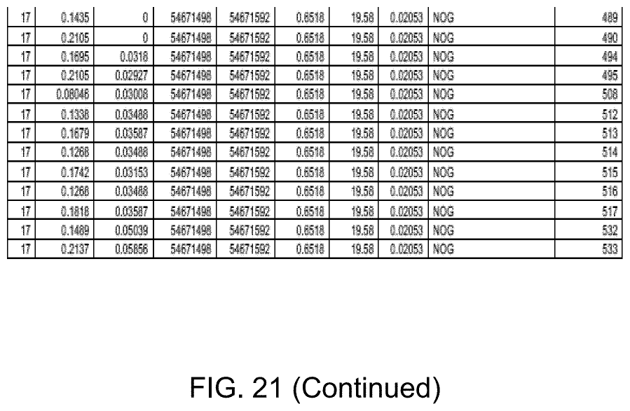

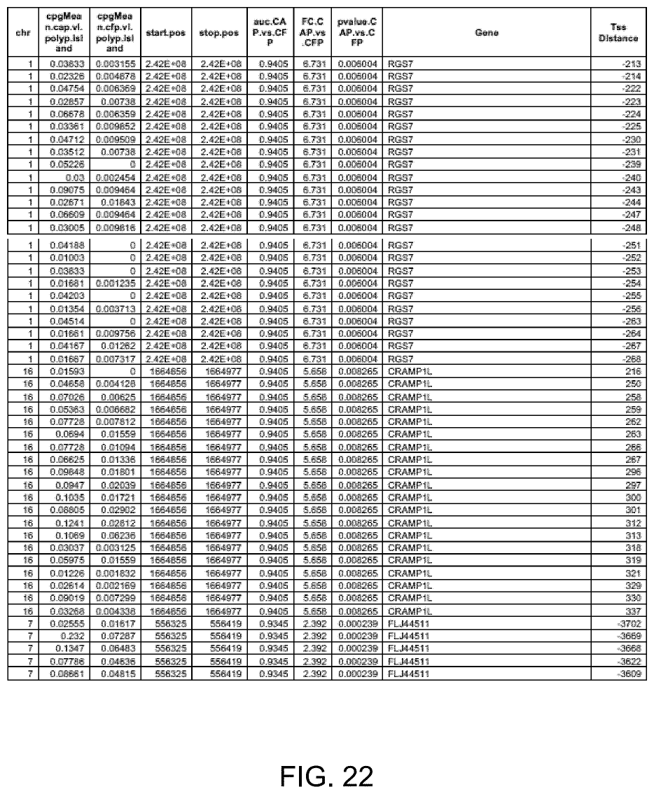

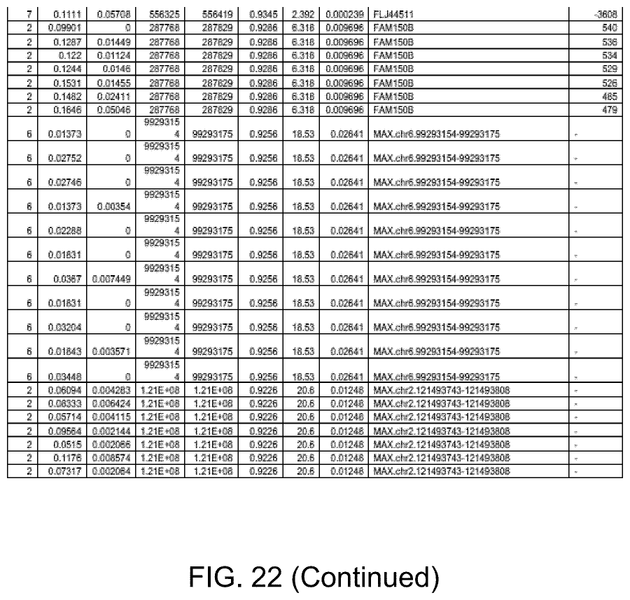

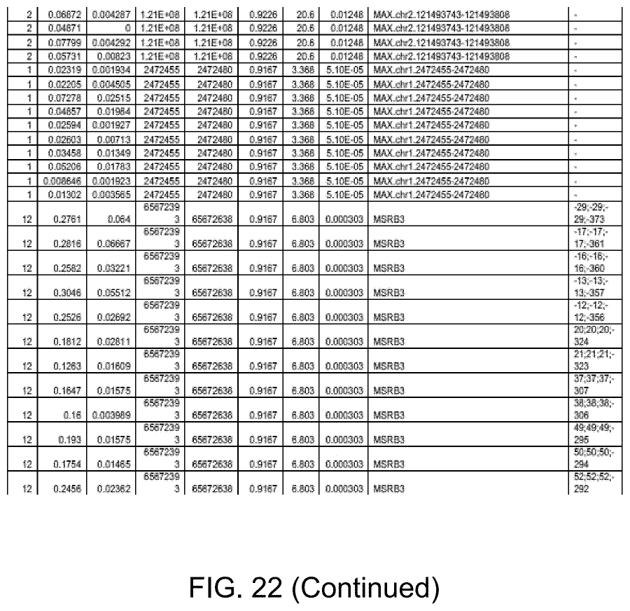

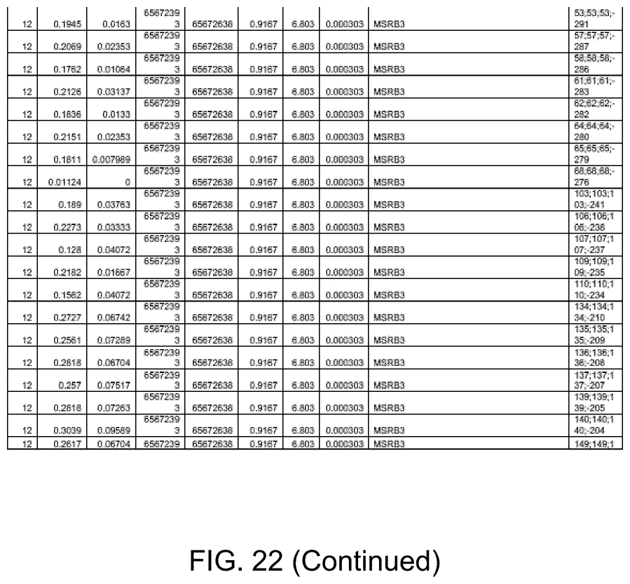

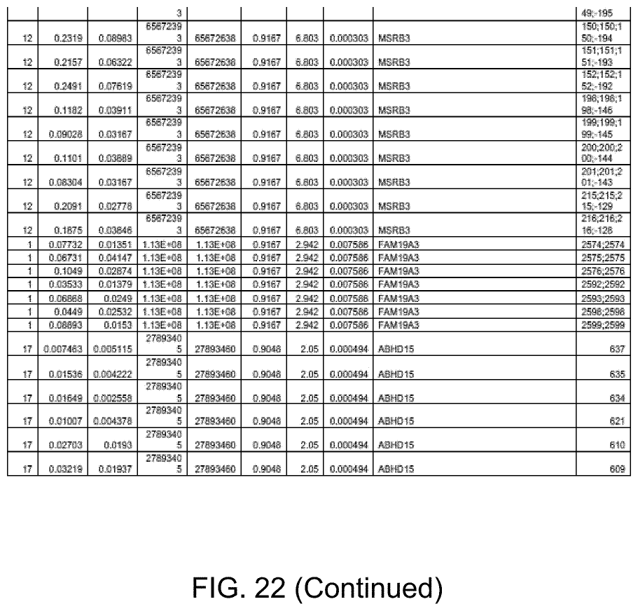

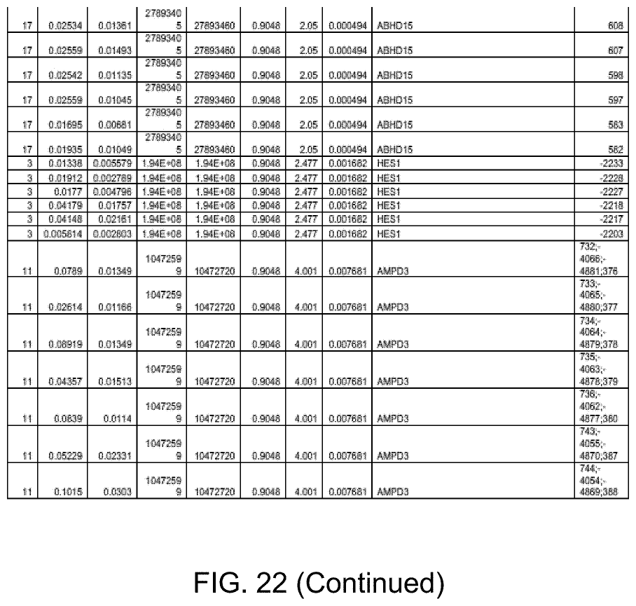

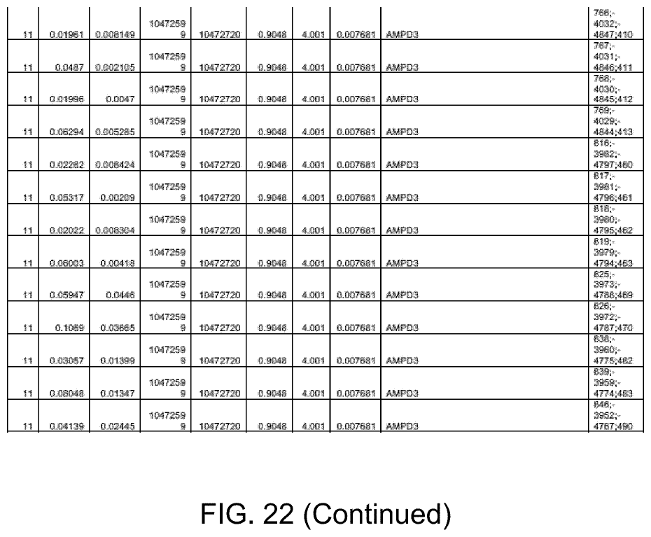

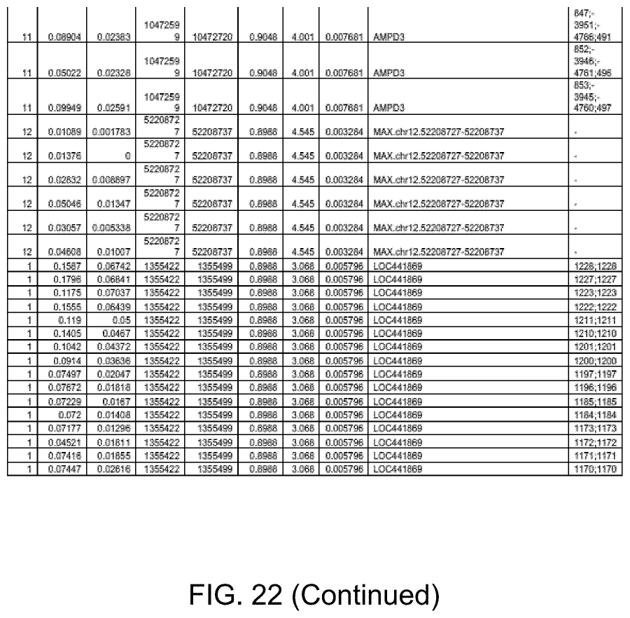

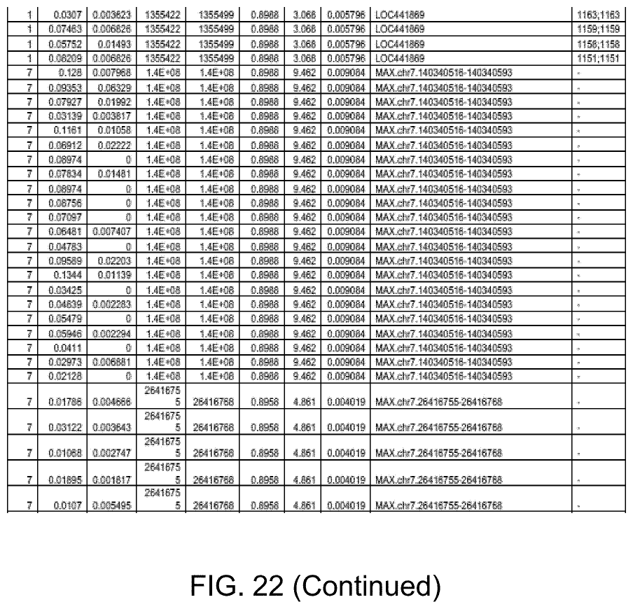

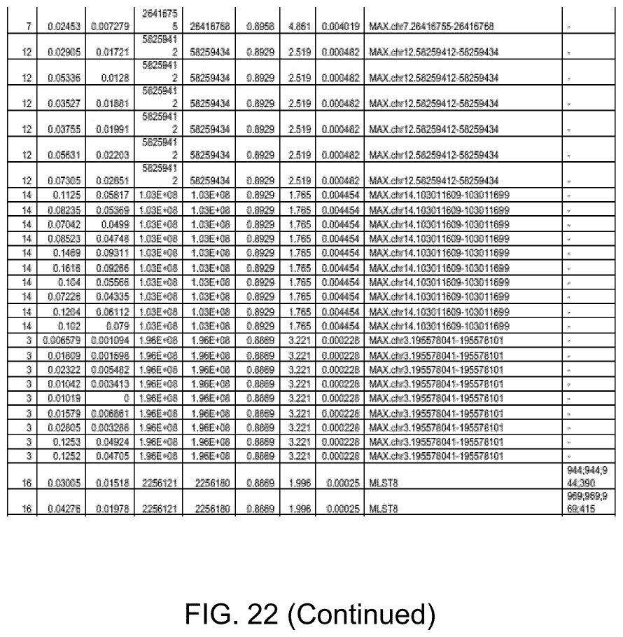

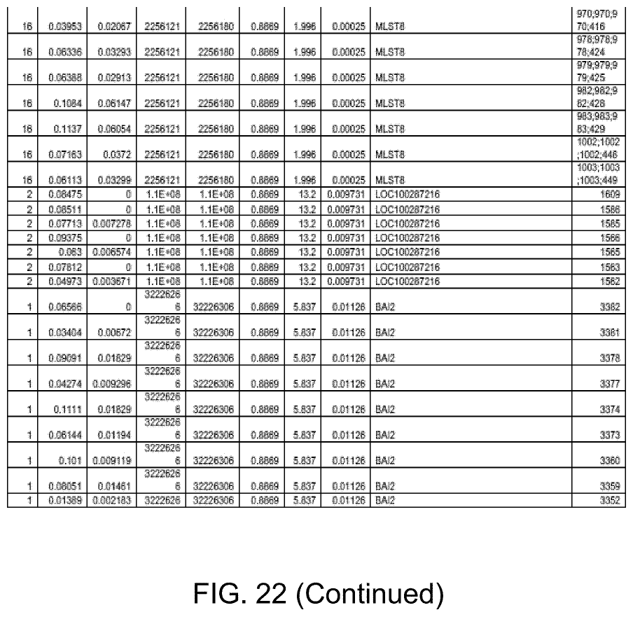

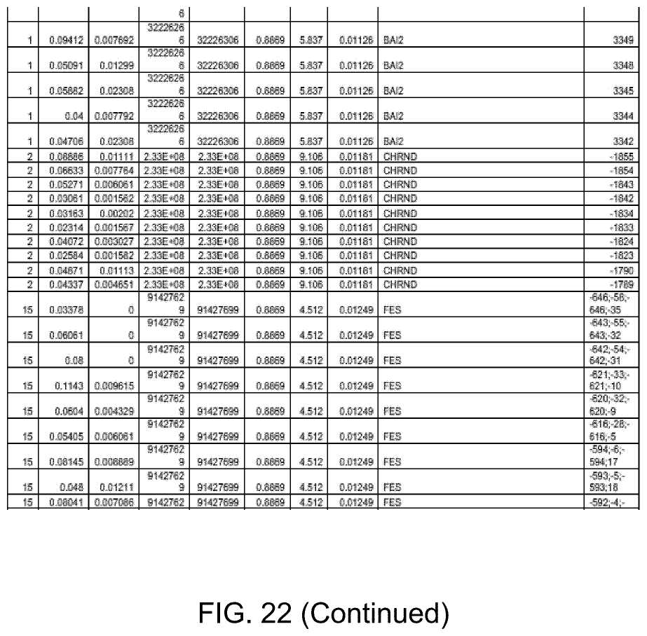

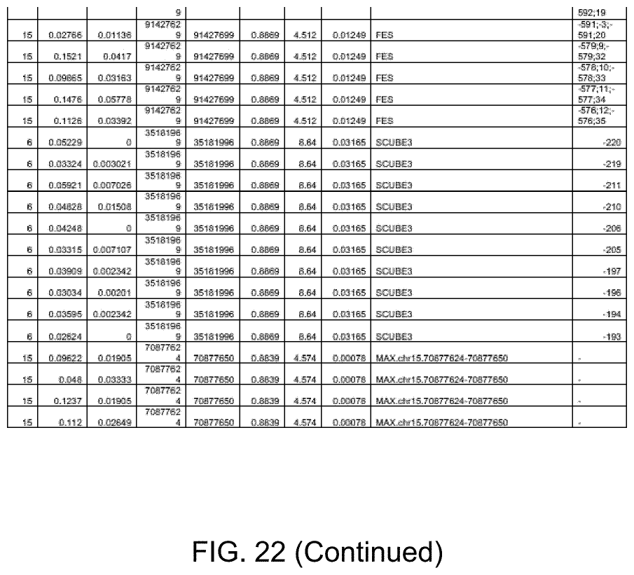

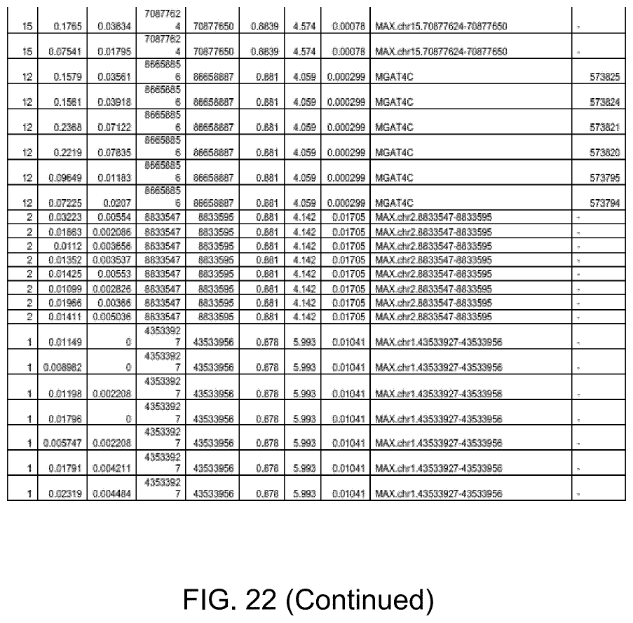

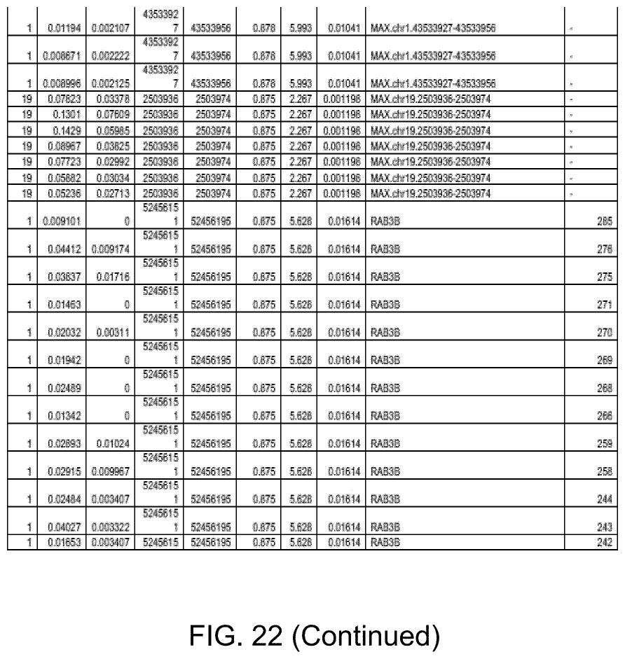

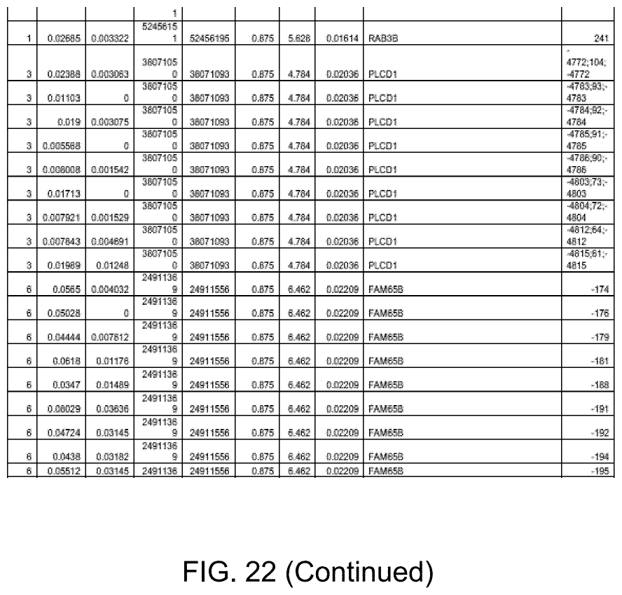

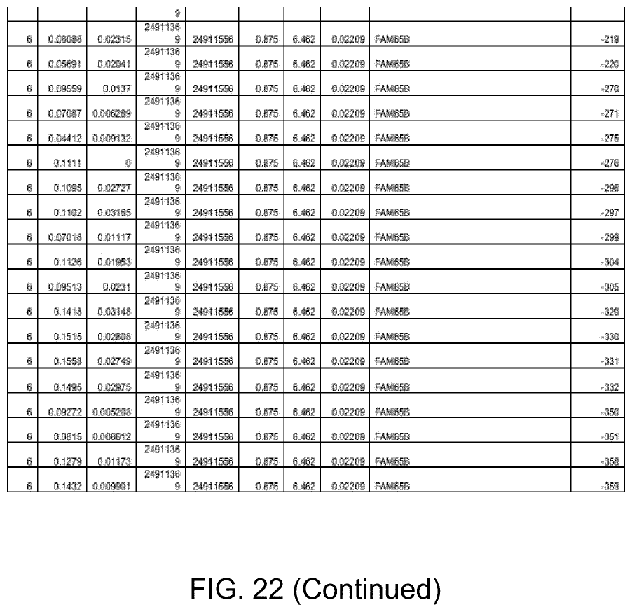

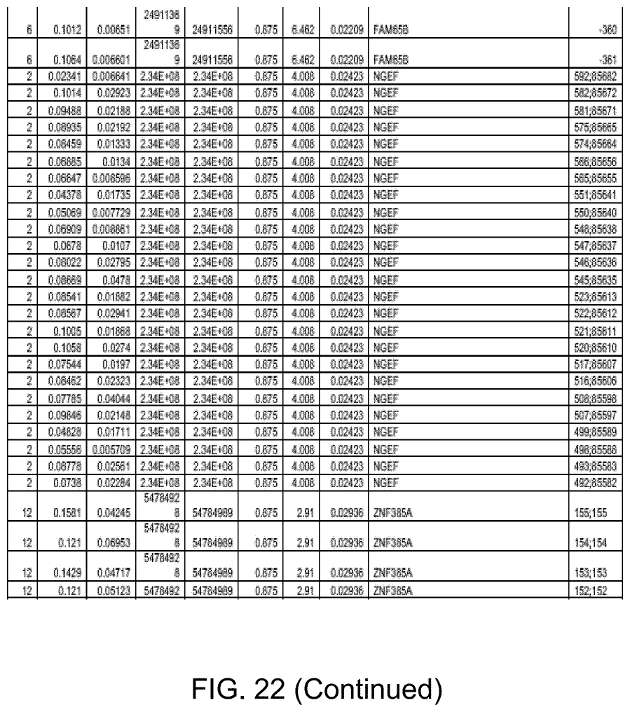

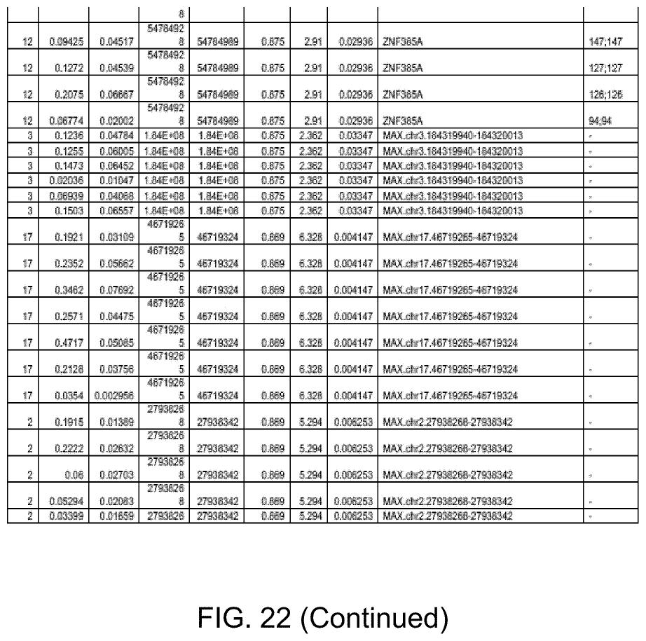

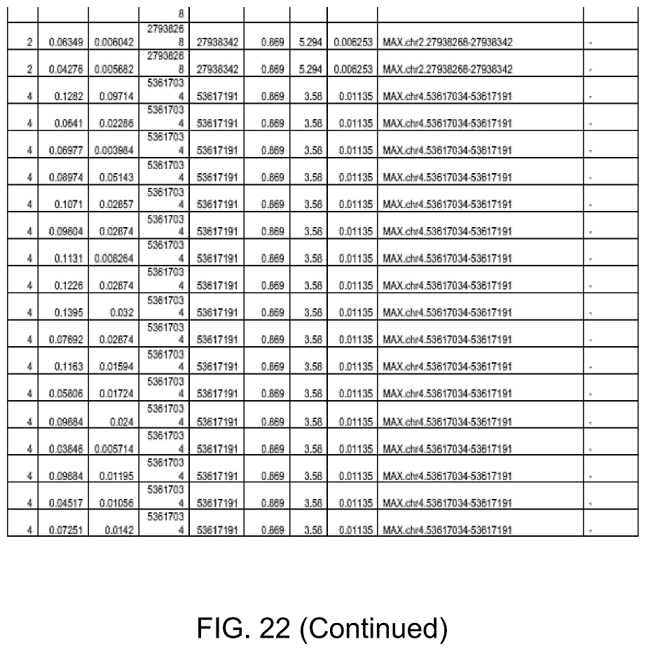

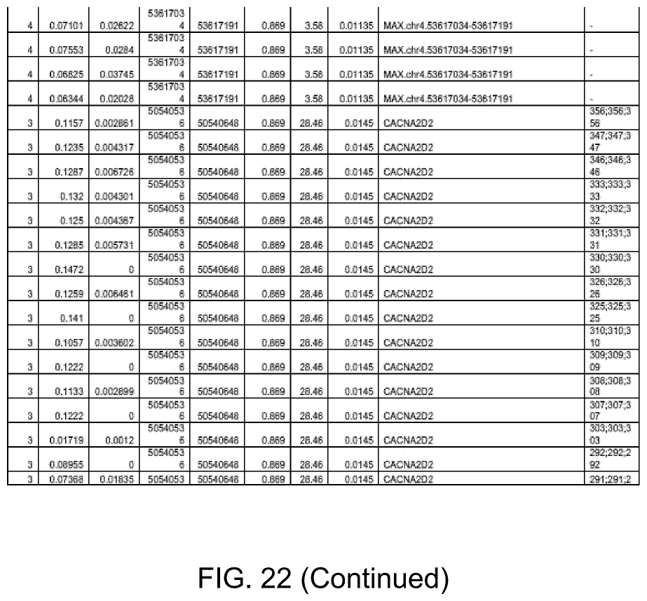

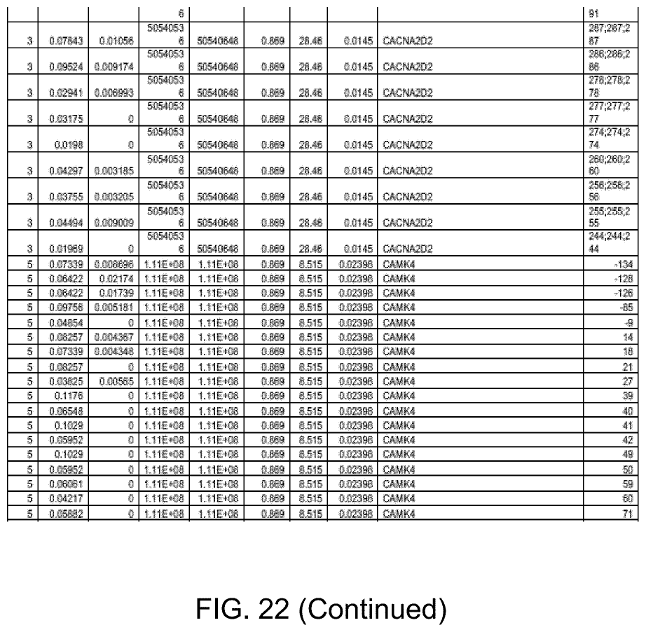

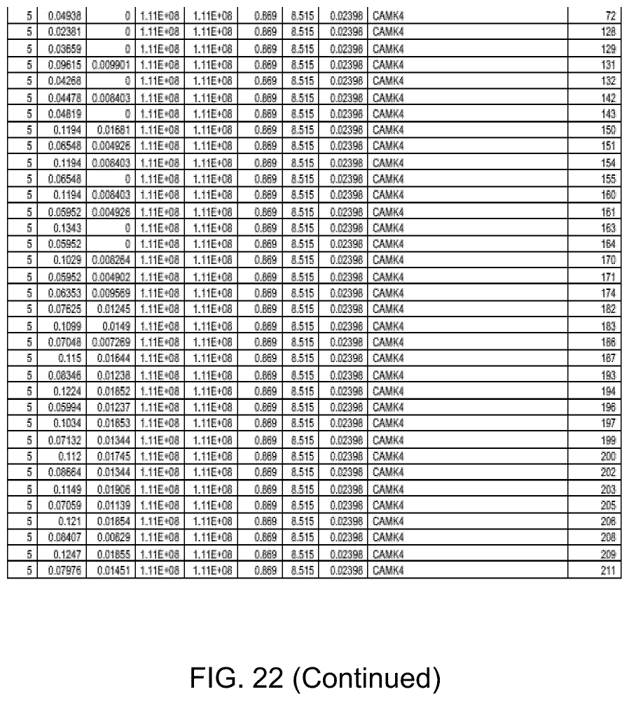

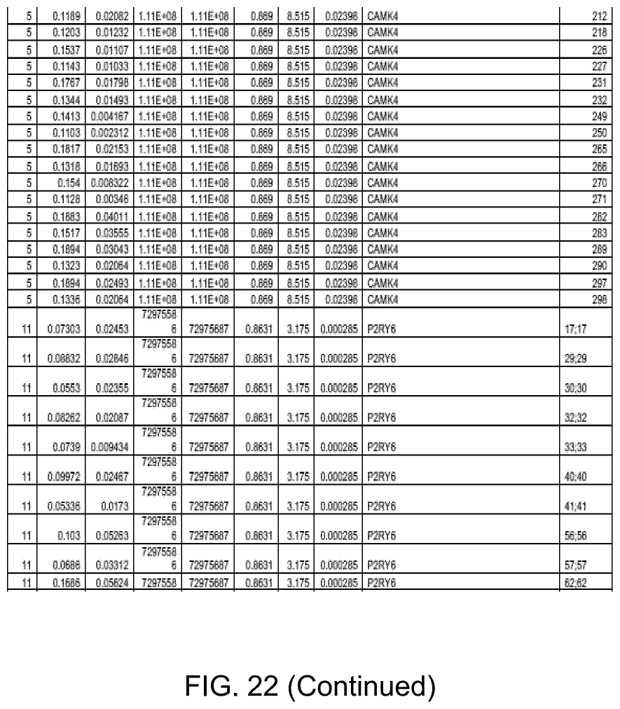

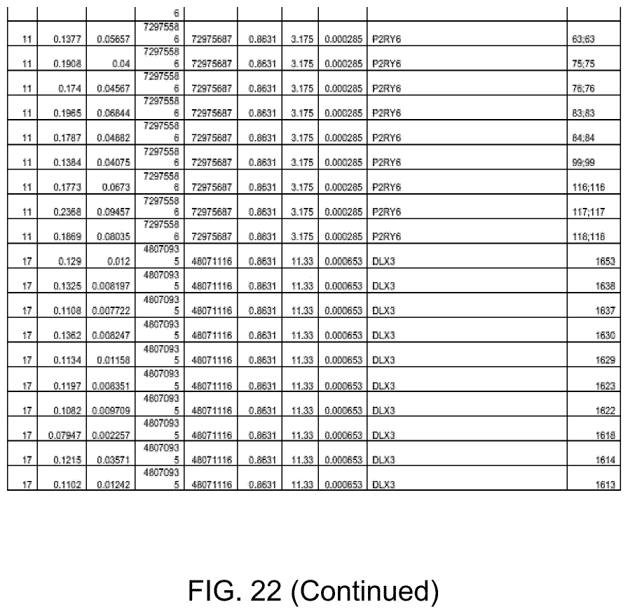

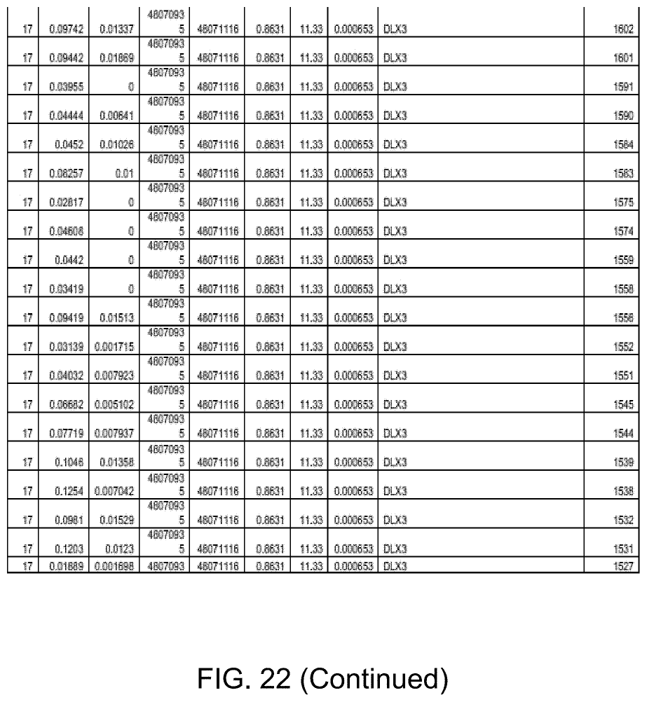

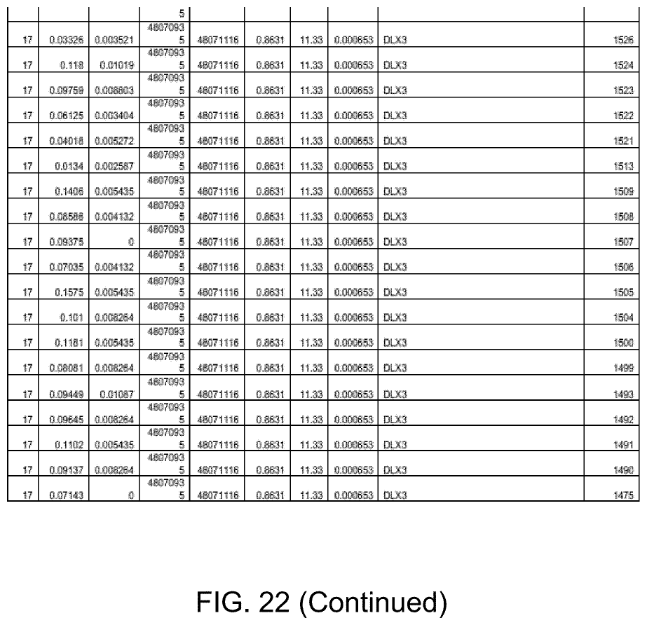

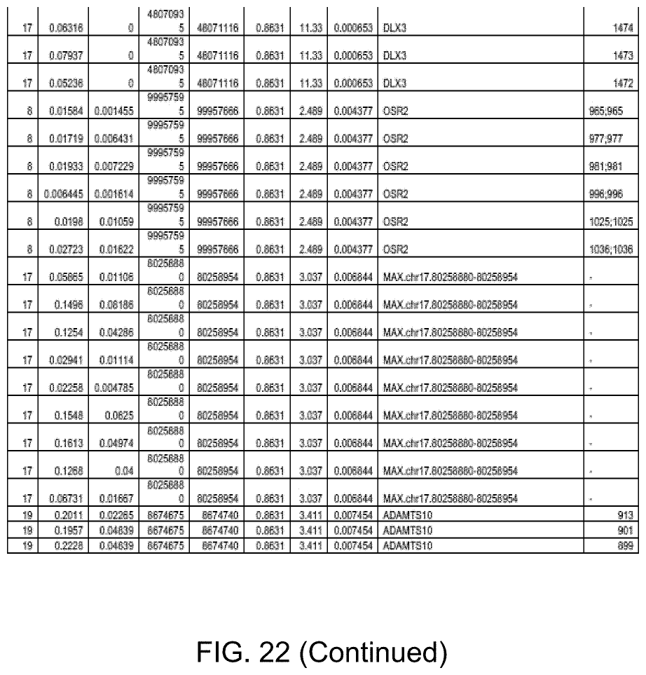

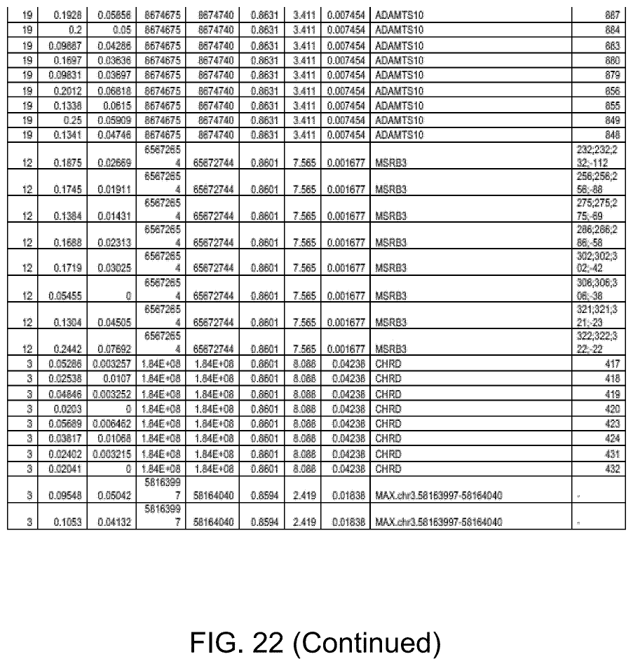

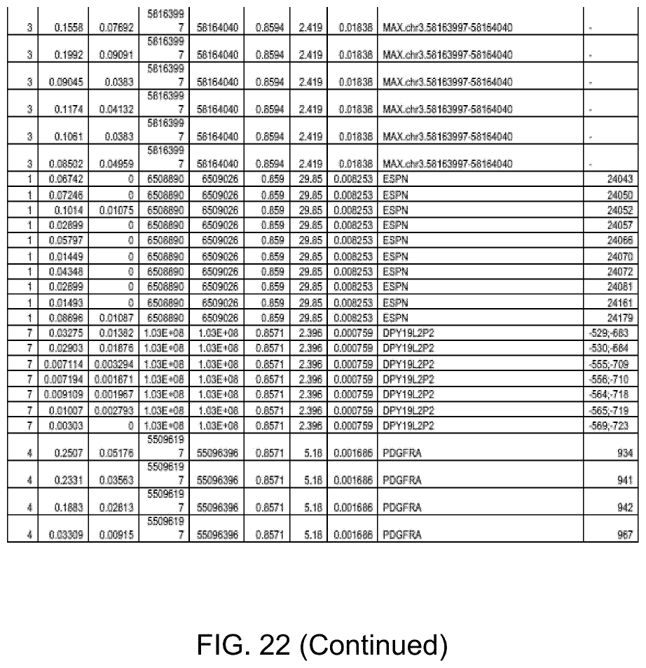

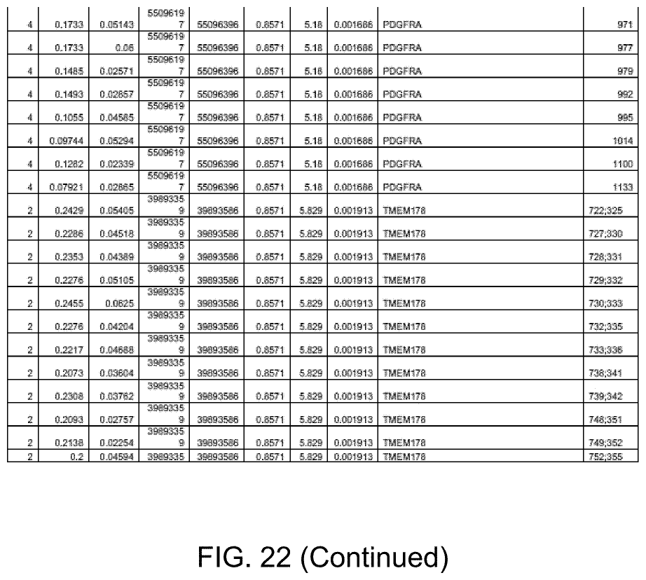

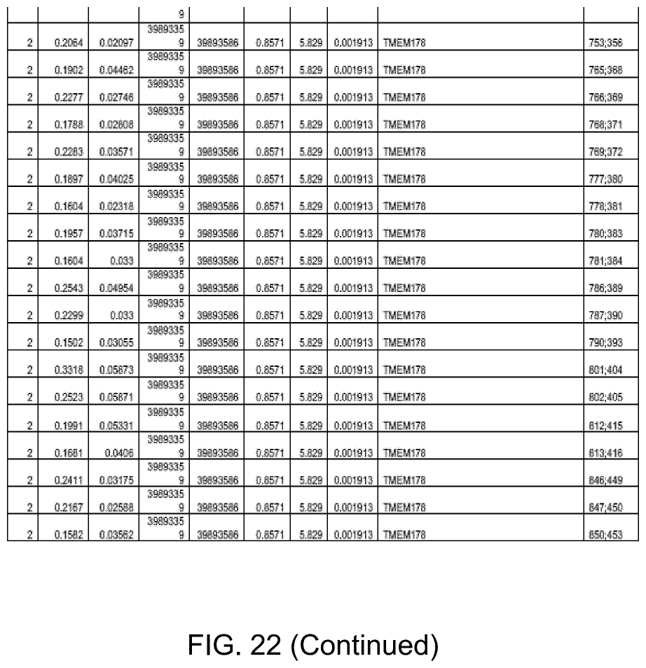

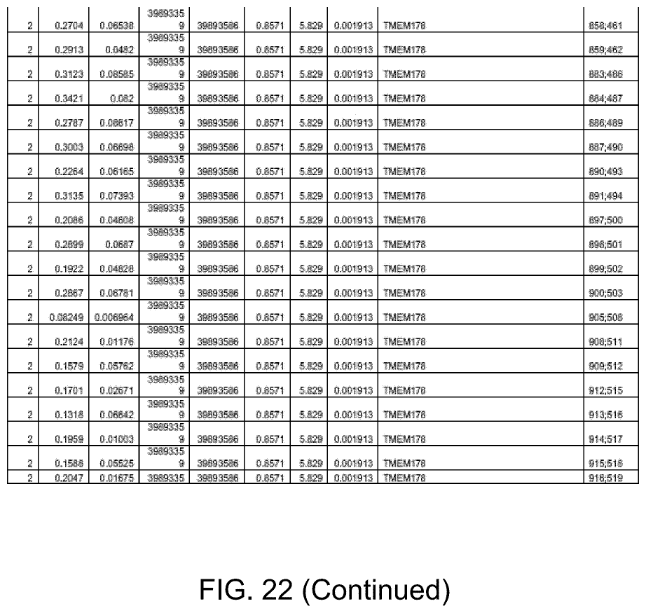

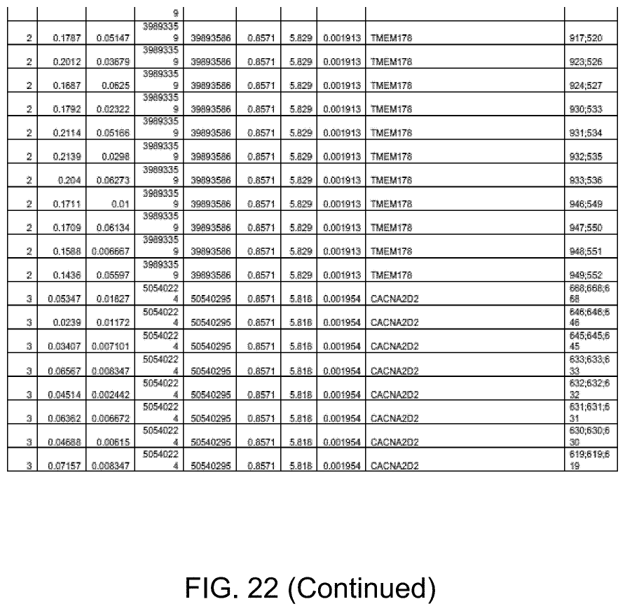

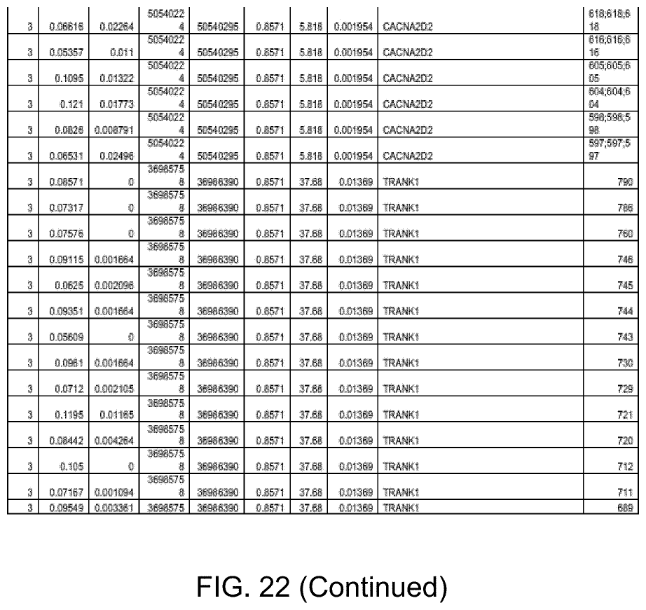

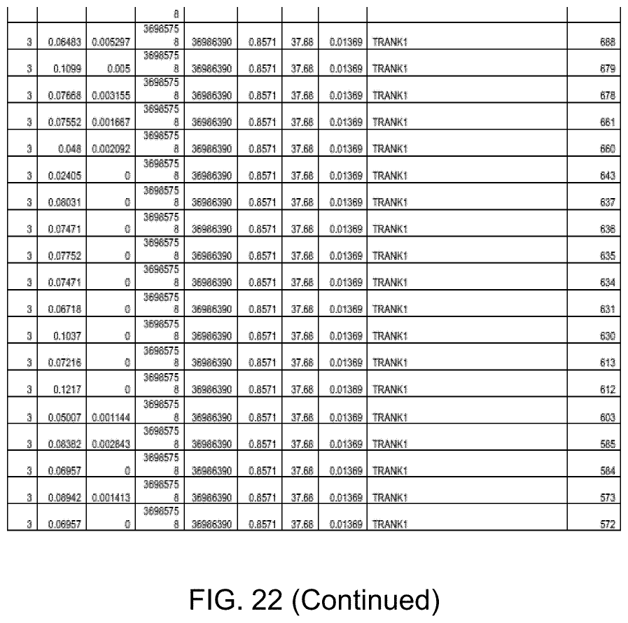

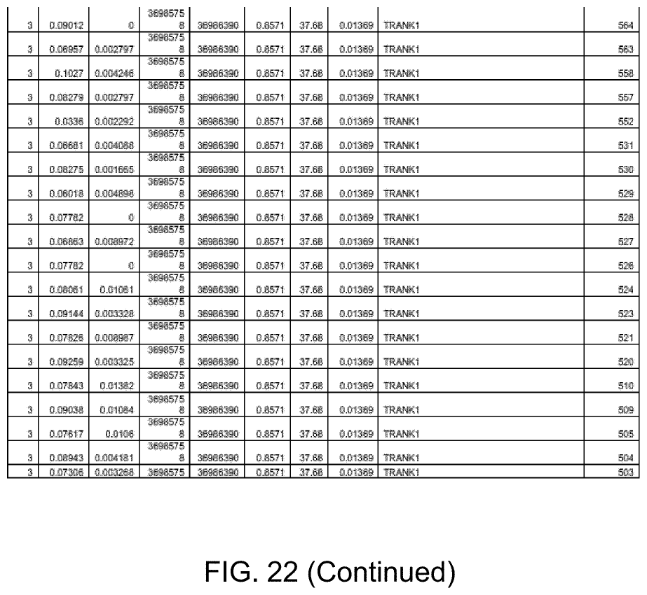

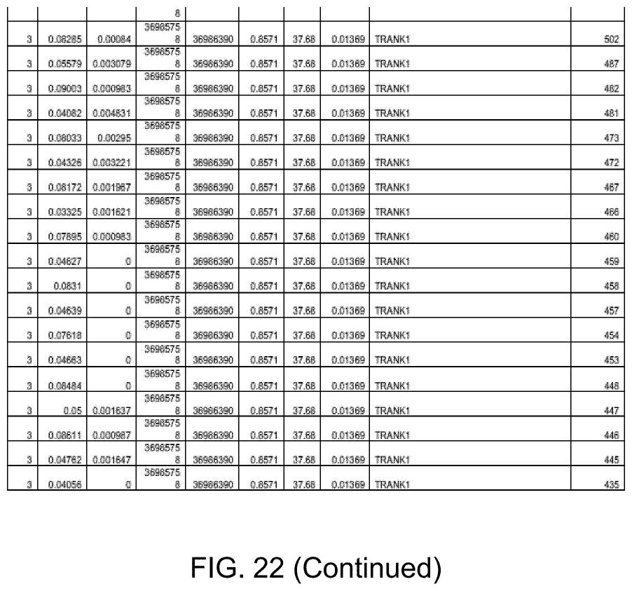

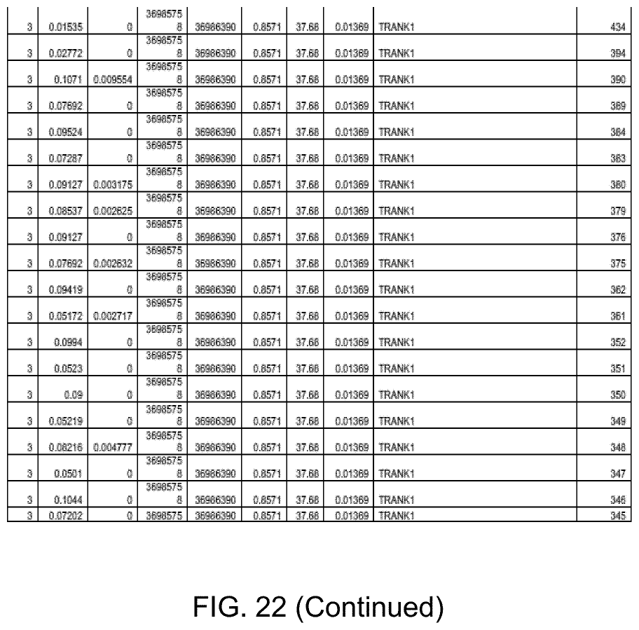

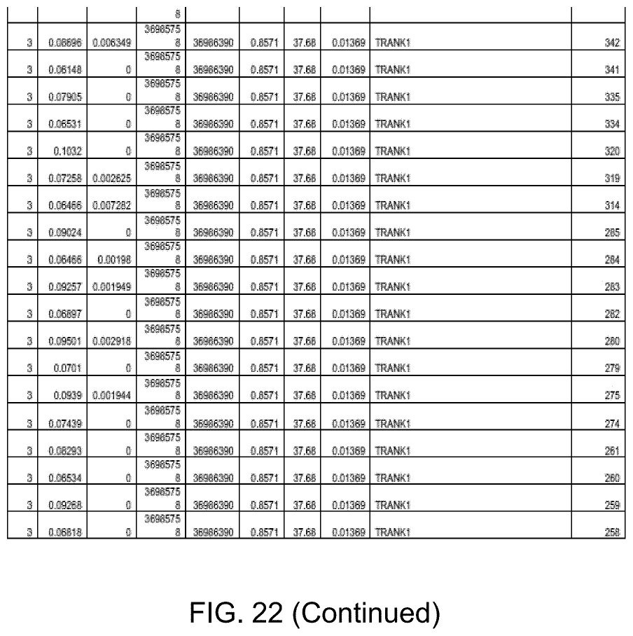

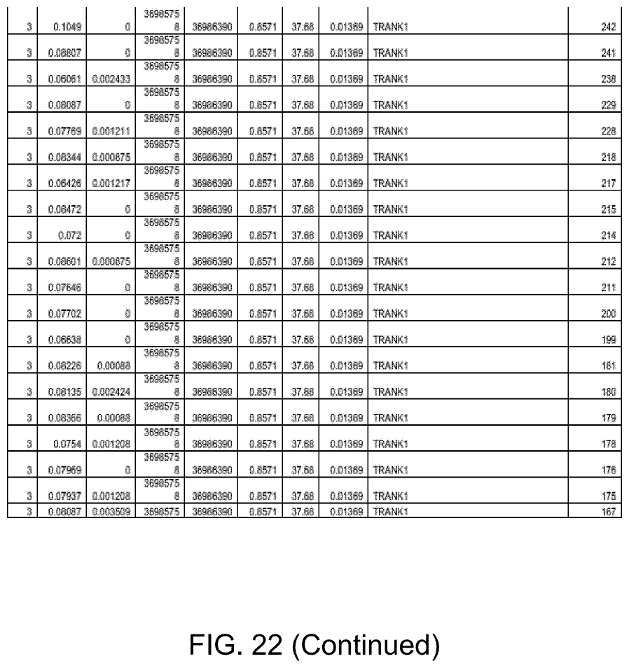

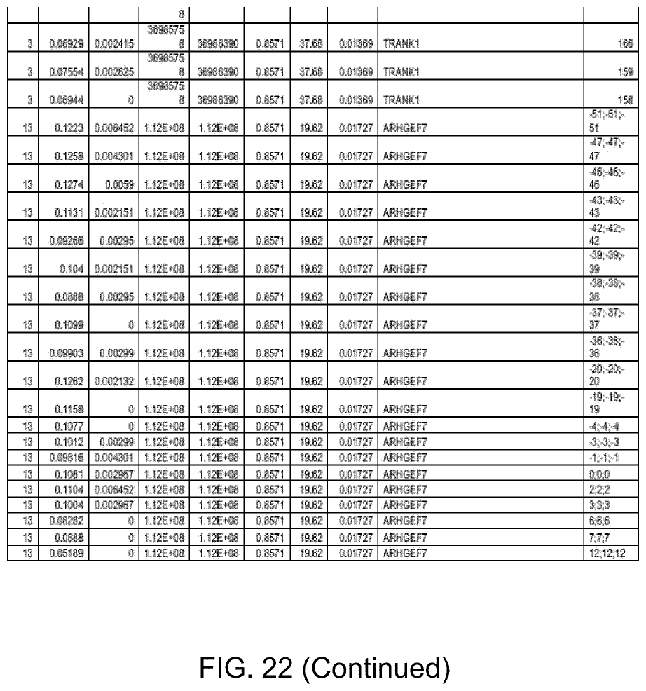

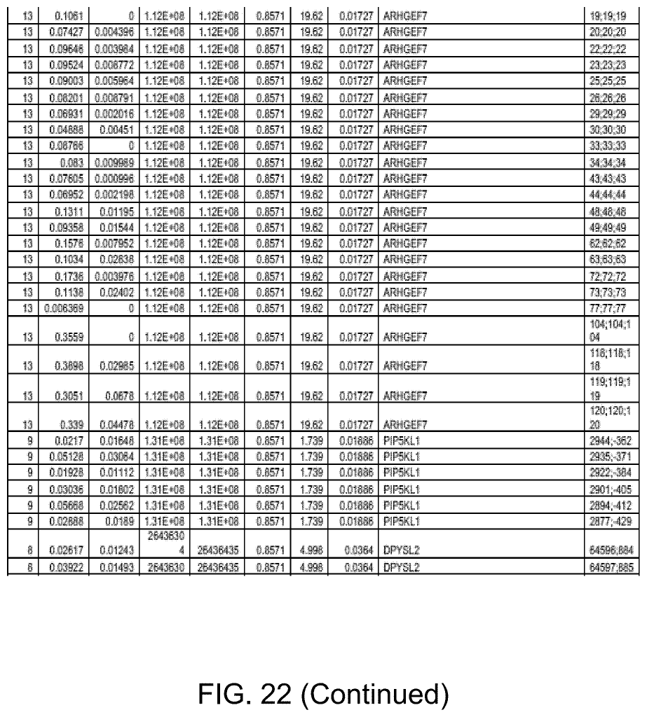

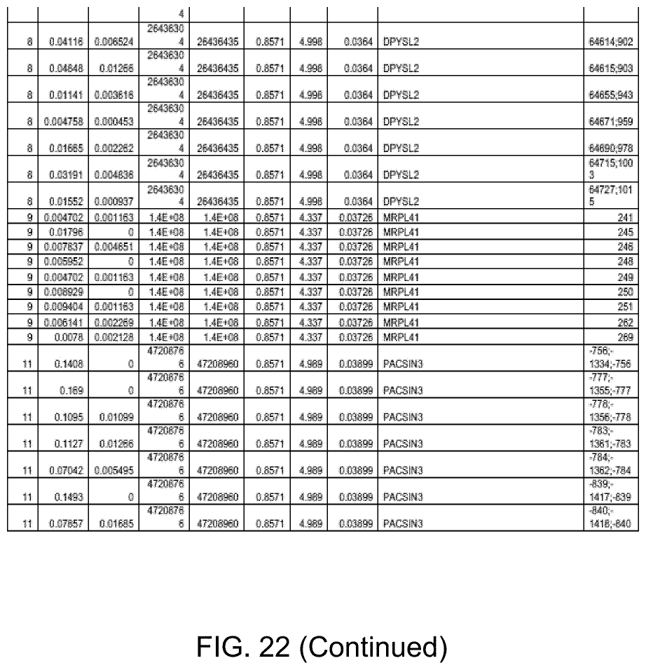

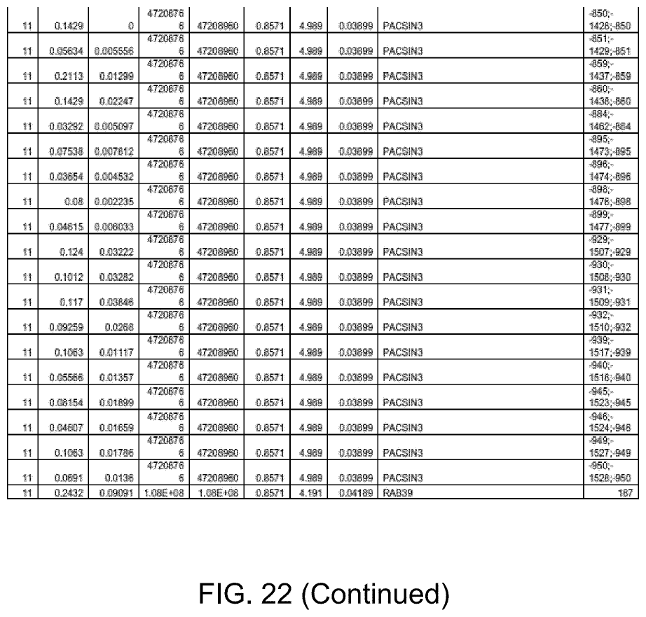

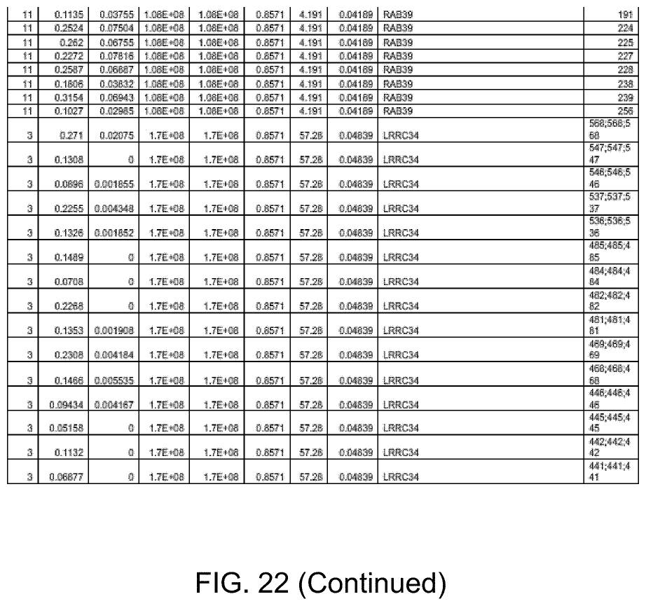

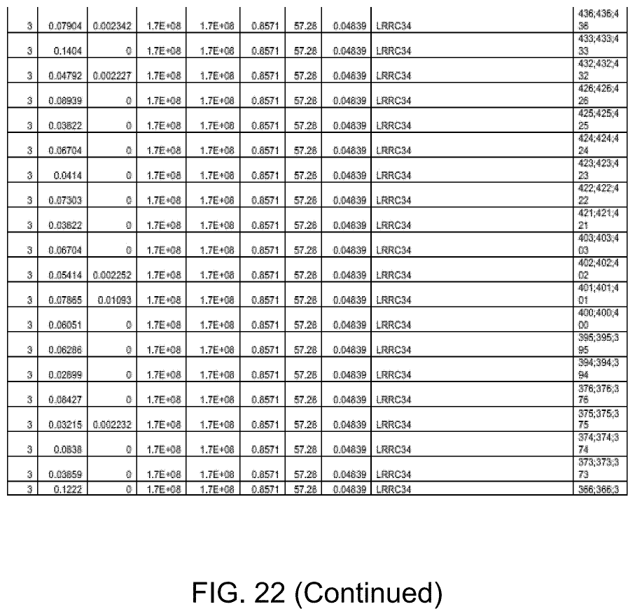

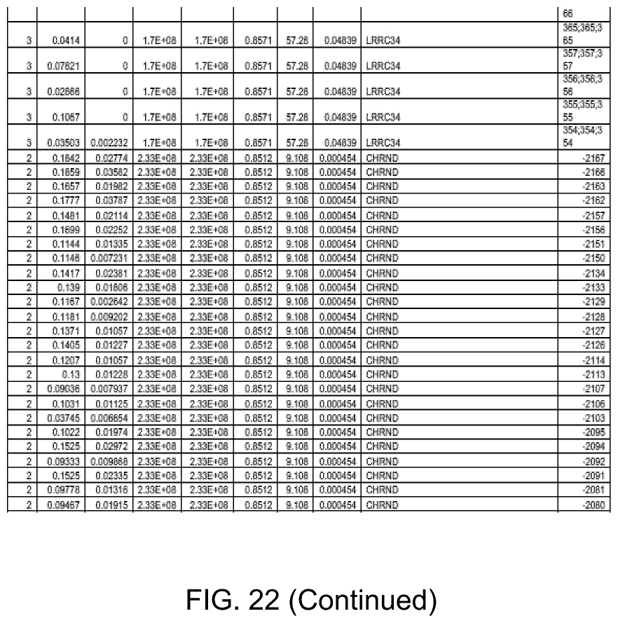

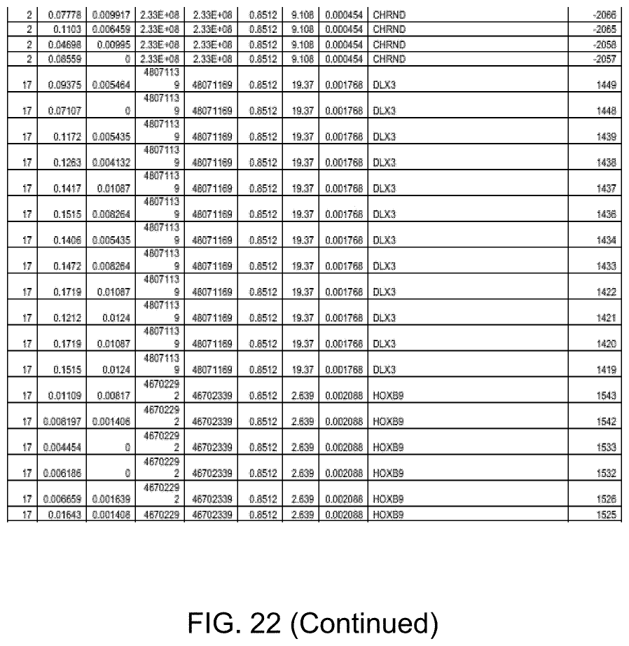

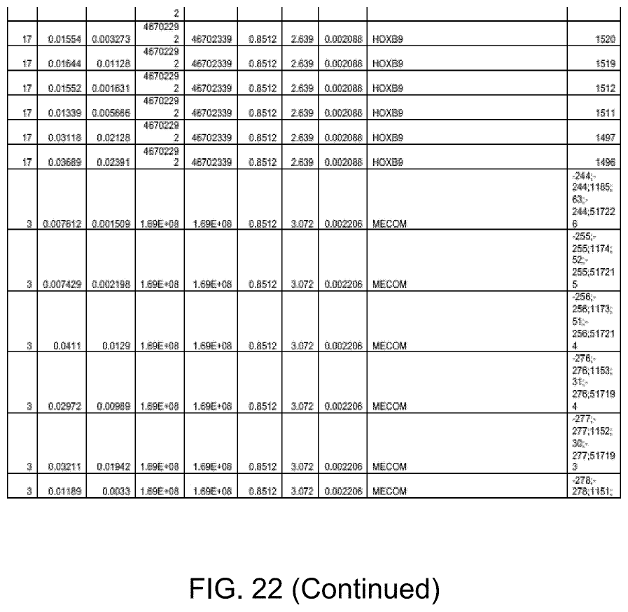

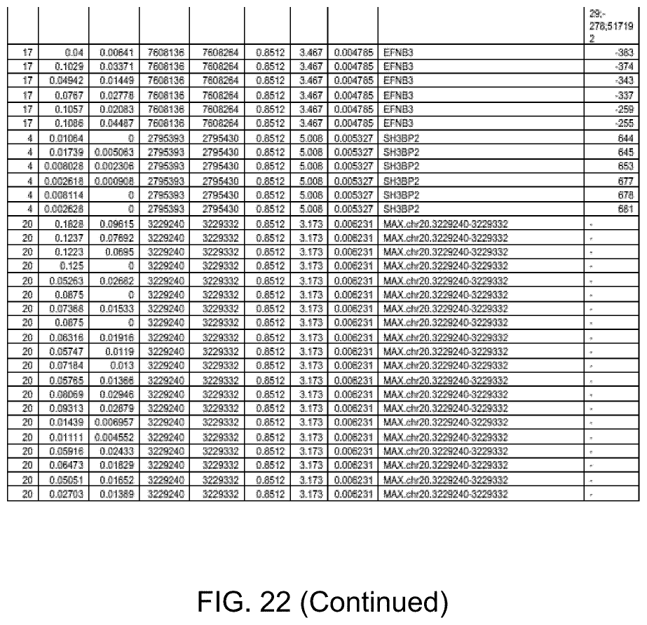

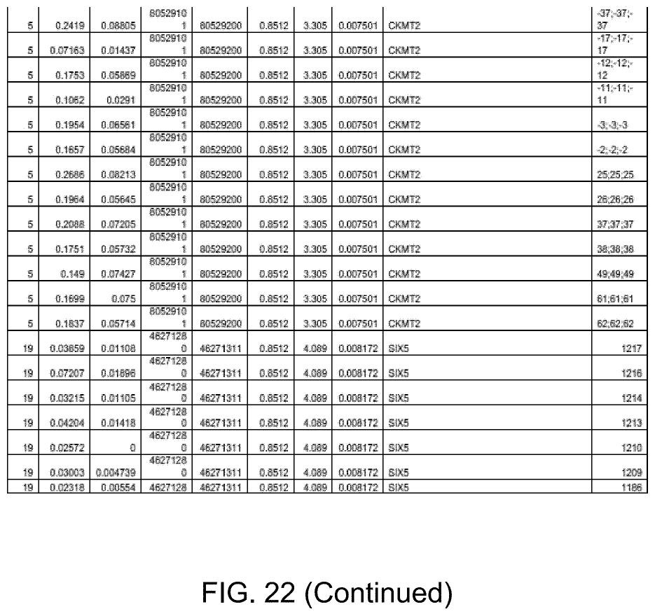

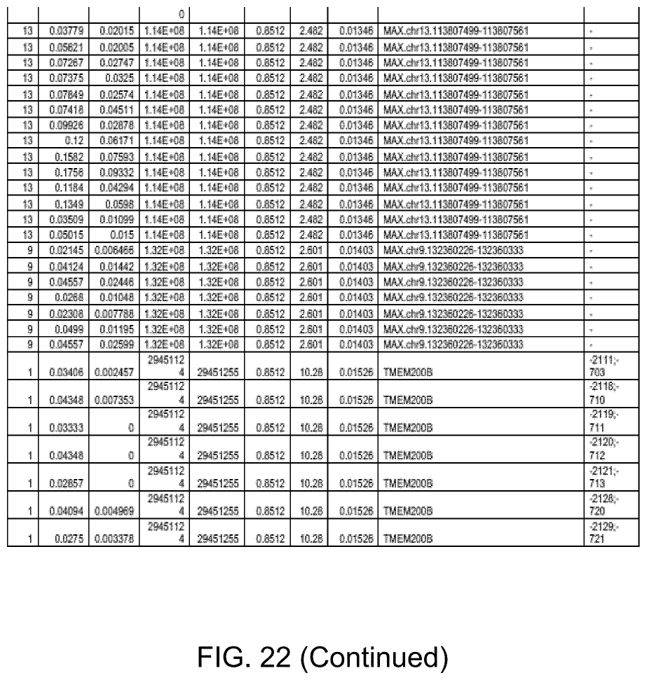

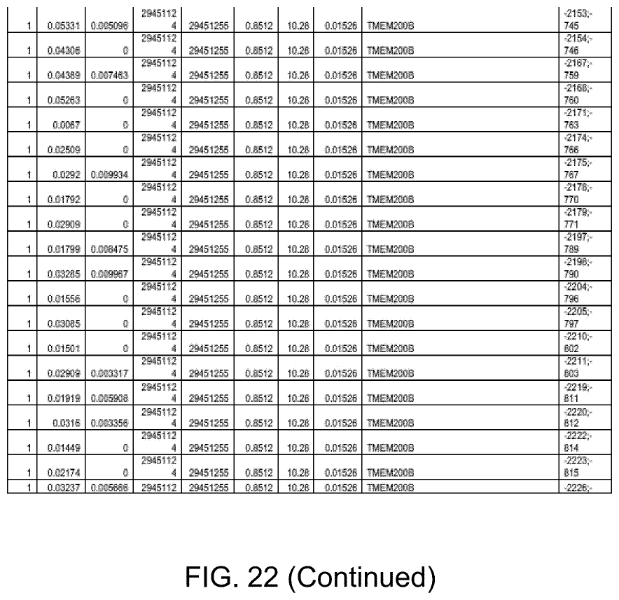

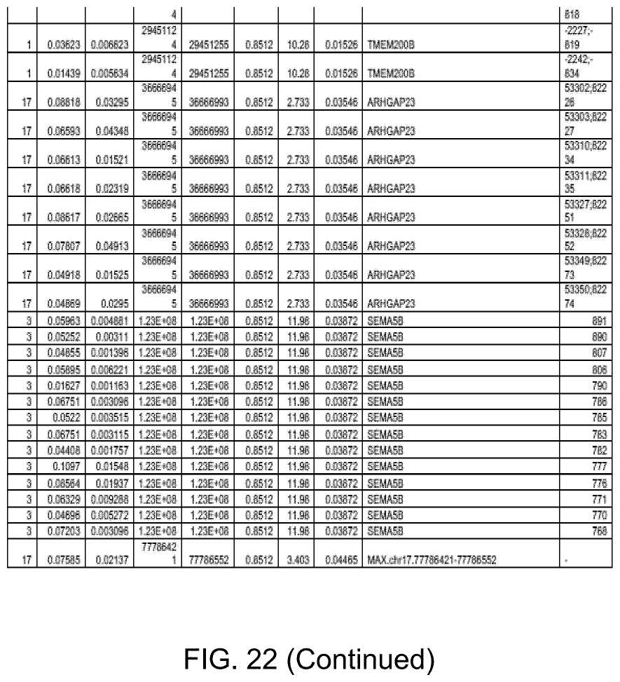

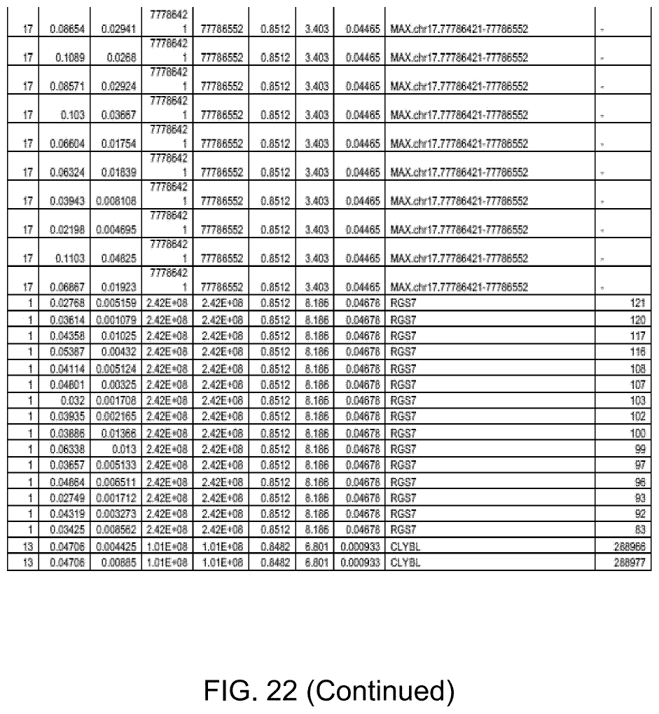

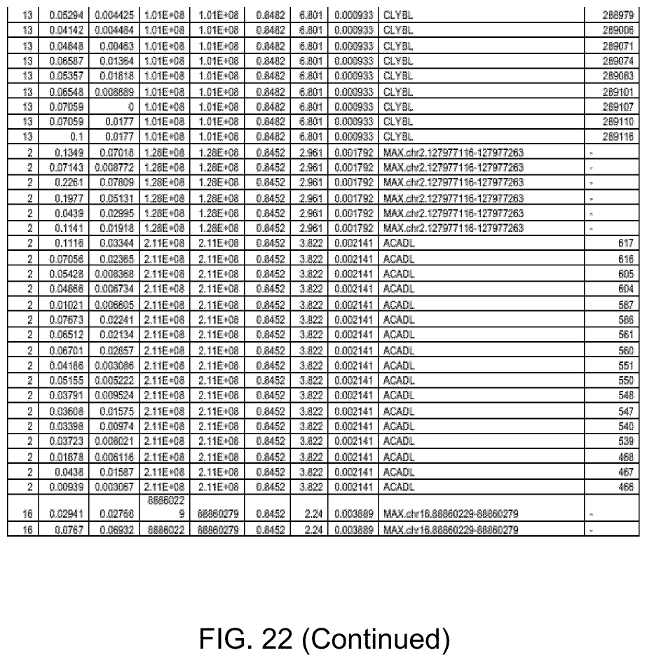

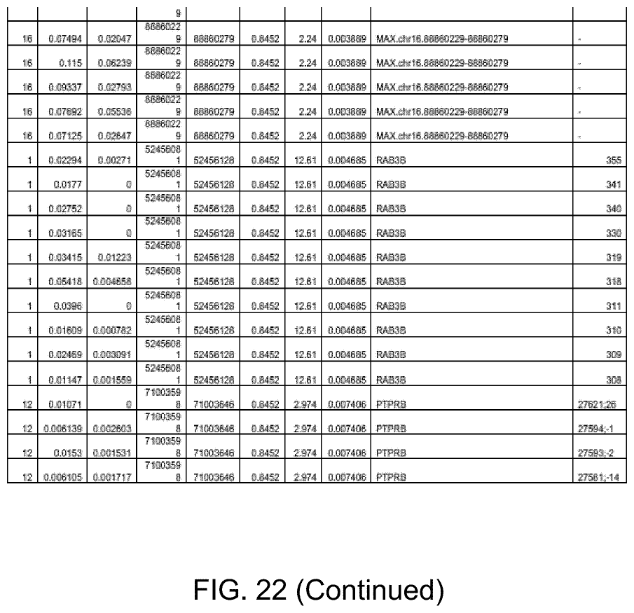

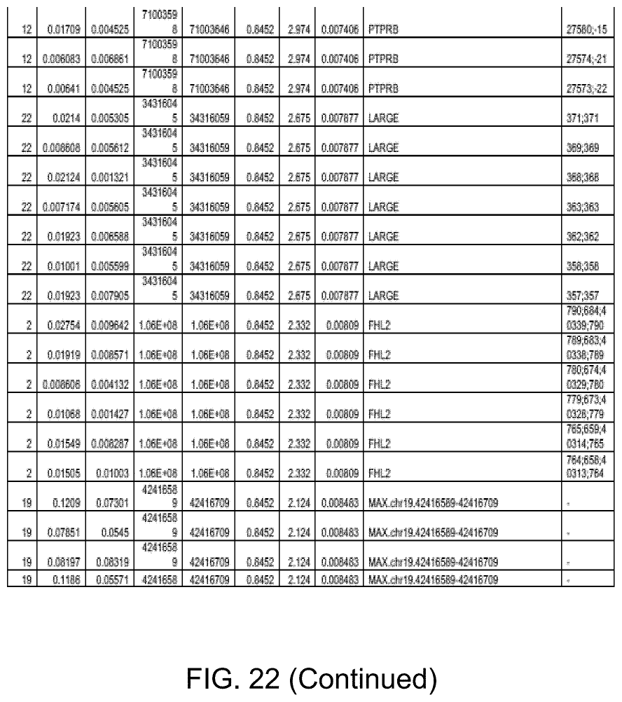

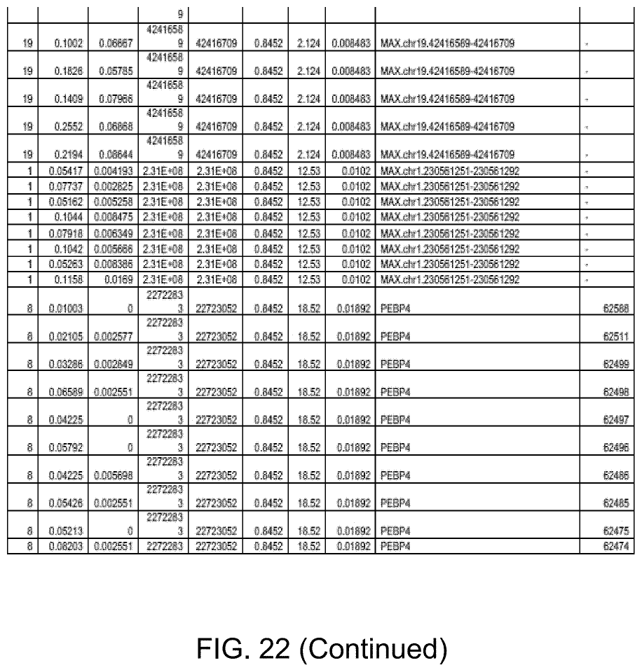

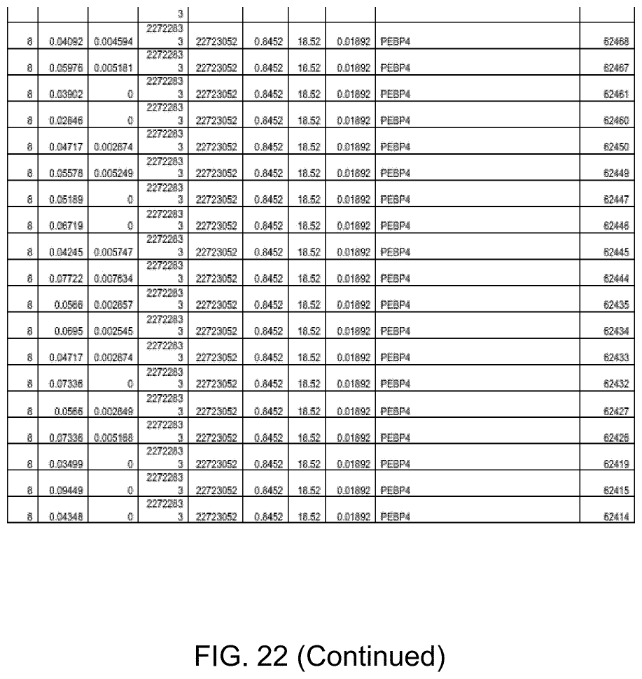

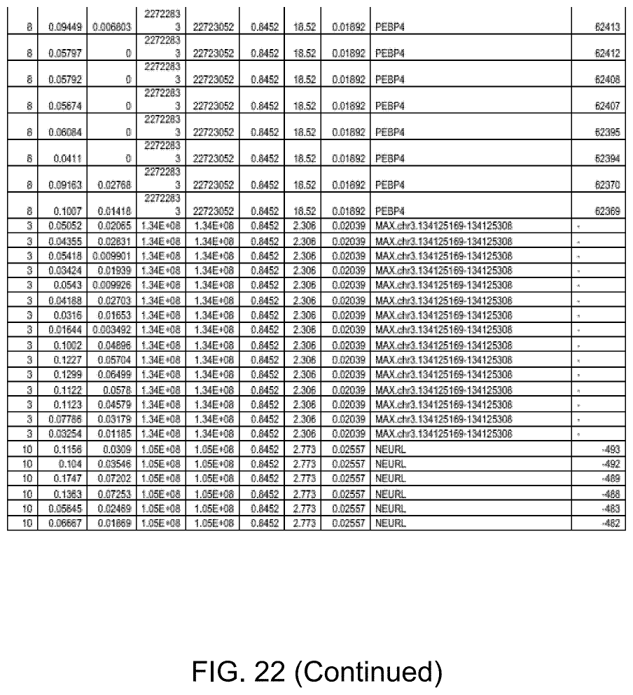

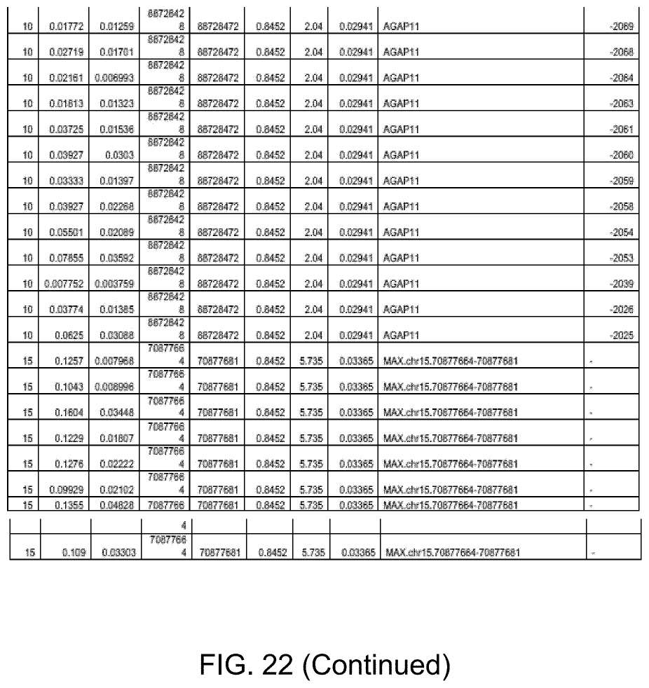

In some cases, a molecular profile that can be used as described herein to determine whether a polyp is likely to recur and/or likely to progress to a cancer can be a molecular profile that includes (a) one or more somatic variations in one or more (e.g., at least one, two, three, four, five, six, seven, eight, nine, ten, eleven, twelve, 13, 14, 15, 20, 25, 30, 35, 40, 45, 50, 60, 70, 80, 90, 100, 125, 150, 175, or 200) of the nucleic acids of Group A, (b) increased expression of one or more (e.g., at least one, two, three, four, five, six, seven, eight, nine, ten, eleven, twelve, 13, 14, 15, 20, 25, 30, 35, 40, 45, 50, 60, 70, 80, 90, 100, 125, 150, 175, or 200) of the nucleic acids of Group B, (c) reduced expression of one or more (e.g., at least one, two, three, four, five, six, seven, eight, nine, ten, eleven, twelve, 13, 14, 15, 20, 25, 30, 35, 40, 45, 50, 60, 70, 80, 90, 100, 125, 150, 175, or 200) of the nucleic acids of Group C, and/or (d) hypermethylation of one or more (e.g., at least one, two, three, four, five, six, seven, eight, nine, ten, eleven, twelve, 13, 14, 15, 20, 25, 30, 35, 40, 45, 50, 60, 70, 80, 90, or 100) of the nucleic acids of Group D. The nucleic acids of Group A are as set forth in Table 3 and FIG. 13. The nucleic acids of Group B are as set forth in FIG. 14, FIG. 15, Table 4, Table 6, and Table 12. The nucleic acids of Group C are as set forth in FIG. 14, FIG. 15, Table 4, Table 6, and Table 12. The nucleic acids of Group D are as set forth in FIG. 21, FIG. 22, Table 12, and Table 13. For example, a molecular profile that can be used as described herein to determine whether a polyp is likely to recur and/or likely to progress to a cancer can be a molecular profile that includes one or more somatic variations in at least 6 of the nucleic acids of Group A, (b) increased expression of at least 5 of the nucleic acids of Group B, reduced expression of at least 1 of the nucleic acids of Group C, and hypermethylation of at least 4 of the nucleic acids of Group D.

In some cases, when a mammal (e.g., a human) is identified as having a polyp that is likely to recur and/or likely to progress to a cancer based, at least in part, on the molecular profile of the polyp as described herein, the presence of a polyp that is likely to recur and/or likely to progress to a cancer can be confirmed using one or more additional diagnostic techniques. Examples of diagnostic techniques that can be used to identify the presence of a polyp that is likely to recur and/or likely to progress to a cancer can include, without limitation, an analysis of histology and degree of dysplasia in the polyp (e.g., via hematoxylin and eosin staining of tissue from a polyp), and flow cytometry (e.g., to assess ploidy).

A mammal (e.g., a human) having one or more polyps (e.g., one or more colon polyps) can be administered, or instructed to self-administer, any one or more (e.g., 1, 2, 3, 4, 5, 6, or more) polyp treatments and/or interventions. Examples of treatments and/or interventions that can be used to treat a mammal having one or more polyps can include, without limitation, removal of the polyp(s) (e.g., by polypectomy (e.g., polypectomy with or without injection of a liquid to lift and isolate the polyp from surrounding tissue), colectomy, laparoscopy, and total proctocolectomy). For example, when a polyp sample from a mammal (e.g., a human) having one or more polyps is used to identify a mammal as having a polyp that is likely to recur and/or likely to progress to a cancer, a treatment can include removing one or more additional polyps (e.g., one or more polyps in addition to any polyp(s) used in the sample) from the mammal. When a treatment includes the removal of colon polyp(s) from a mammal, the treatment can remove at least about 50 percent (e.g., about 50 percent, about 55 percent, about 60 percent, about 70 percent, about 75 percent, about 80 percent, about 85 percent, about 90 percent, about 95 percent, or more).

In cases where a mammal (e.g., a human) is identified as having a polyp that is likely to recur and/or likely to progress to a cancer based, at least in part, on the molecular profile of the polyp as described herein, the mammal also can be selected for more frequent (e.g., additional and/or increased) screenings (e.g., more frequent cancer screening than was performed previously on the mammal). In some cases, a mammal identified as having a polyp that is likely to recur and/or likely to progress to a cancer can be selected for more frequent screenings for the presence or absence of polyps. For example, a mammal identified as having a polyp that is likely to recur and/or likely to progress to a cancer can be selected for more frequent imaging techniques such as using a flexible tube with a light and camera attached to it to visualize internal organs (e.g., colonoscopy, endoscopy, and sigmoidoscopy), computerized tomography (CT) scanning (e.g., CT colonography), x-ray techniques (e.g., barium enema x-ray techniques), more frequent laboratory tests such as stool-based tests (e.g., fecal occult tests and/or assessing stool DNA), and/or more frequent physical examinations (e.g., digital rectal examinations).

In cases where a mammal (e.g., a human) is identified as having a polyp that is likely to recur and/or likely progress to a cancer based, at least in part, on the molecular profile of the polyp as described herein, the mammal also can be administered any one or more (e.g., 1, 2, 3, 4, 5, 6, or more) cancer treatments. A cancer treatment can include any appropriate cancer treatment. In some cases, a cancer treatment can include administering one or more cancer drugs (e.g., chemotherapeutic agents and/or targeted cancer drugs) to a mammal in need thereof. Examples of chemotherapeutic agents that can be administered to a mammal having one or more polyps (e.g., colorectal polyps) include, without limitation, capecitabine, fluorouracil, oxaliplatin, leucovorin, avastin, cetuximab, pembrolizumab, and combinations thereof. In some cases, a cancer treatment can include surgery (e.g., colectomy and/or lymph node removal). In some cases, a cancer treatment can include radiation treatment.

The invention will be further described in the following examples, which do not limit the scope of the invention described in the claims.

### Examples

### Example 1: Molecular Characterization of Colorectal Adenomas with and without Malignancy Reveals Distinguishing Genome, Transcriptome and Methylome Alterations

In order to investigate differences between polyps with and without cancer, CAP and CFP tissues were characterized based on their genetic, expression and methylation patterns. A key element in our approach is the comparison of polyp tissue with and without cancer based on over five years of follow up.

The identification of the molecular profiles that differentiate CAPs from CFPs has the potential to lead to tailored colonoscopy surveillance intervals. Adding defined molecular features to assess a polyp's risk for malignancy will improve the impact of surveillance on CRC prevention. Ultimately, definition of molecular alterations linked with progression of polyps to cancer could lead to modifiable targets for chemoprevention or other preventive interventions, and may also serve up candidate markers for screening.

### Results

Time Lapse Model: Colorectal Polyps with and without Cancer

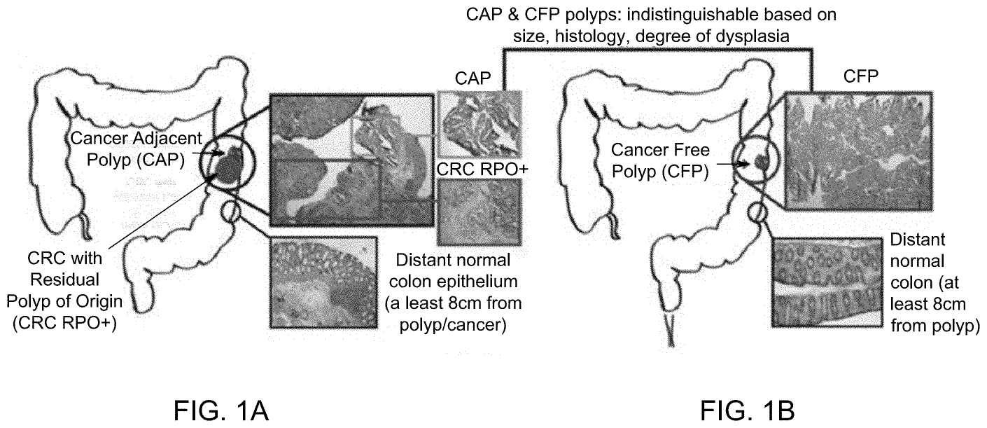

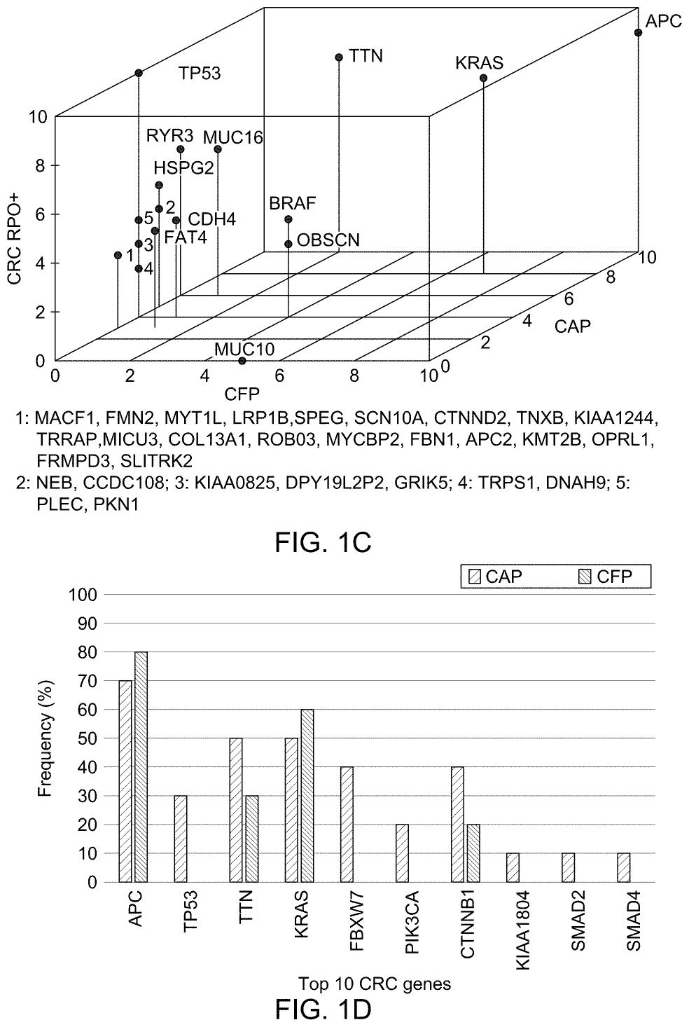

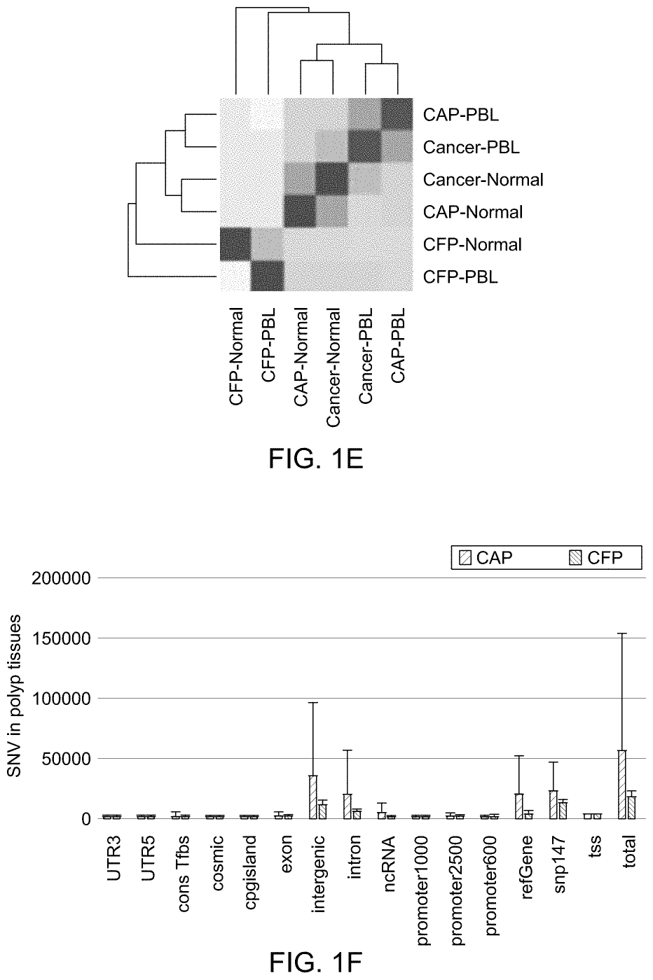

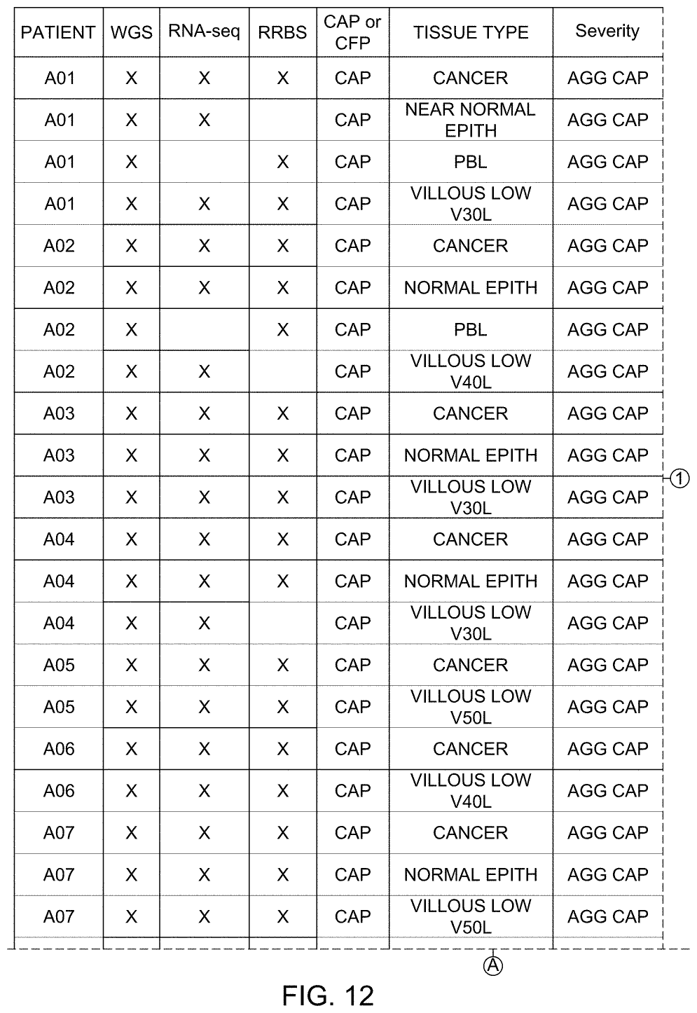

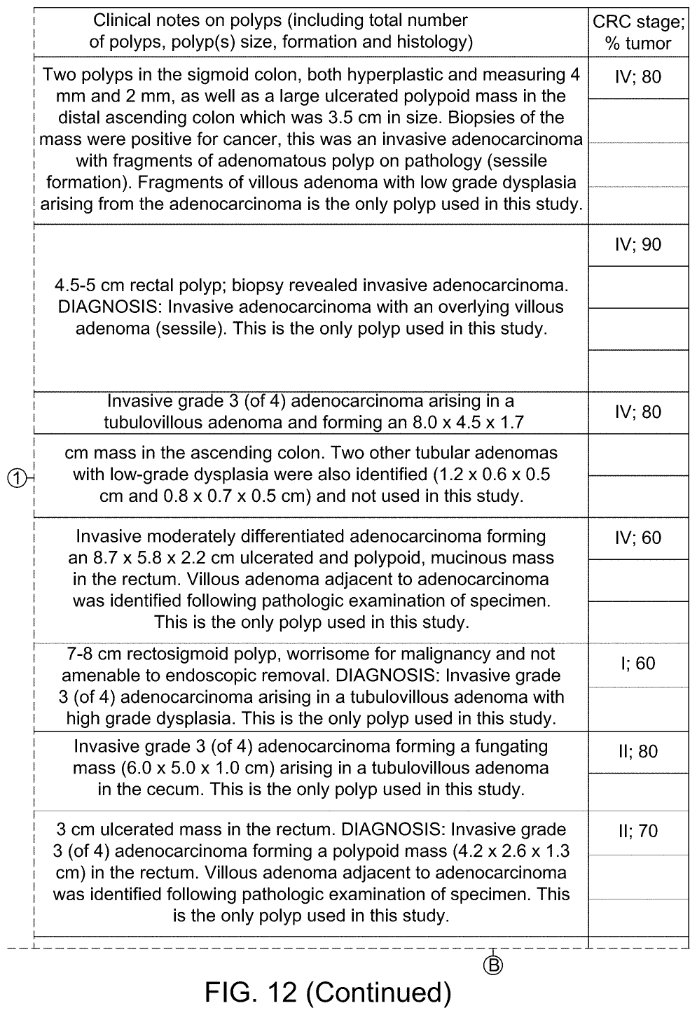

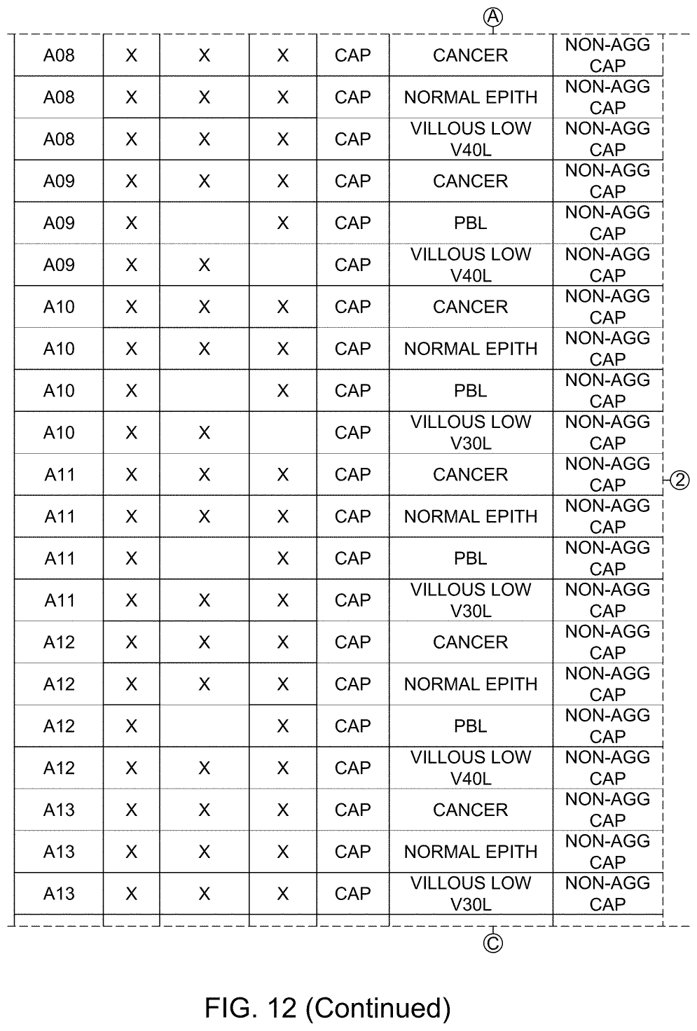

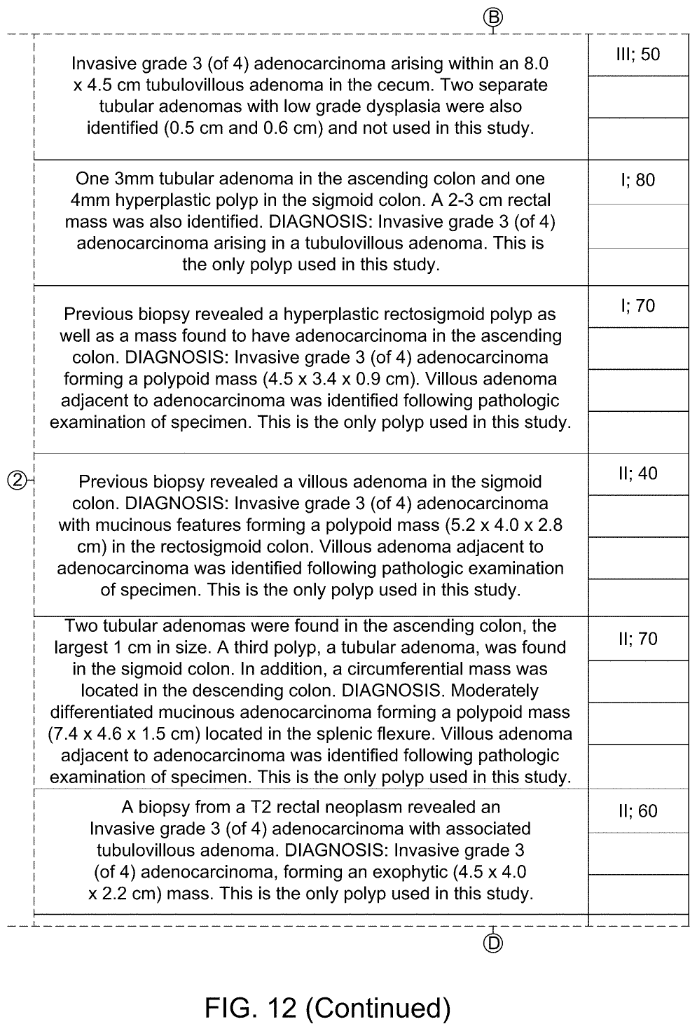

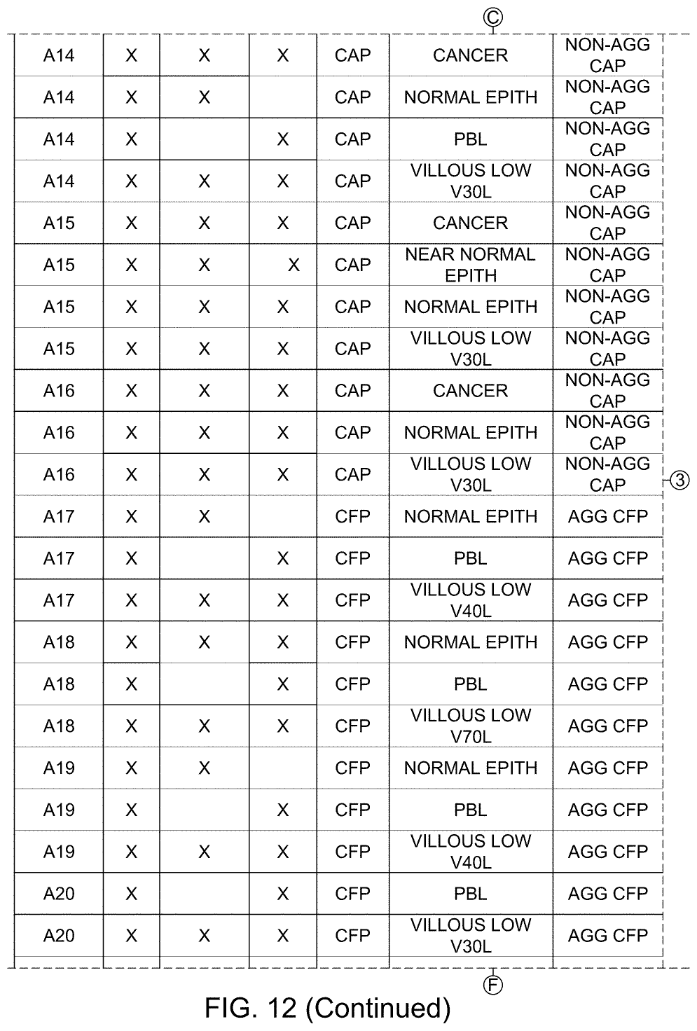

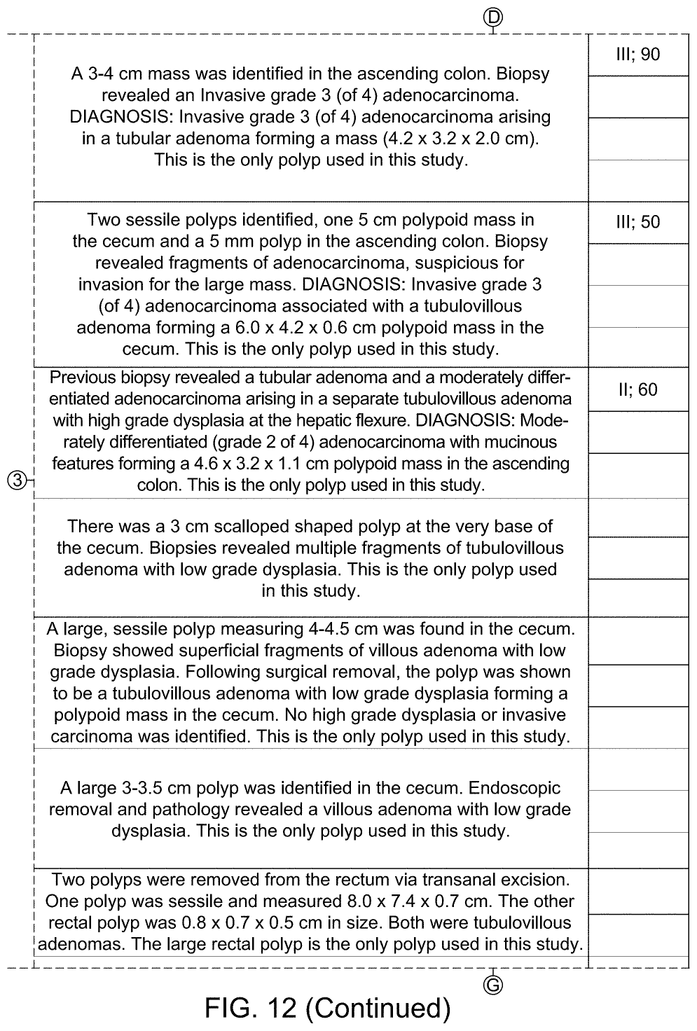

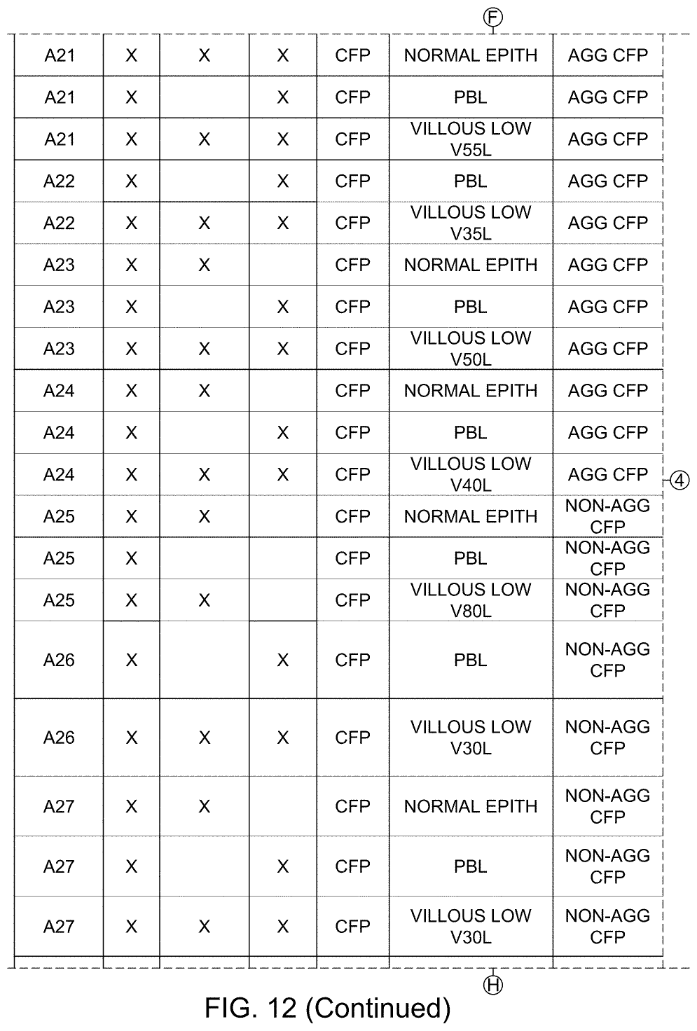

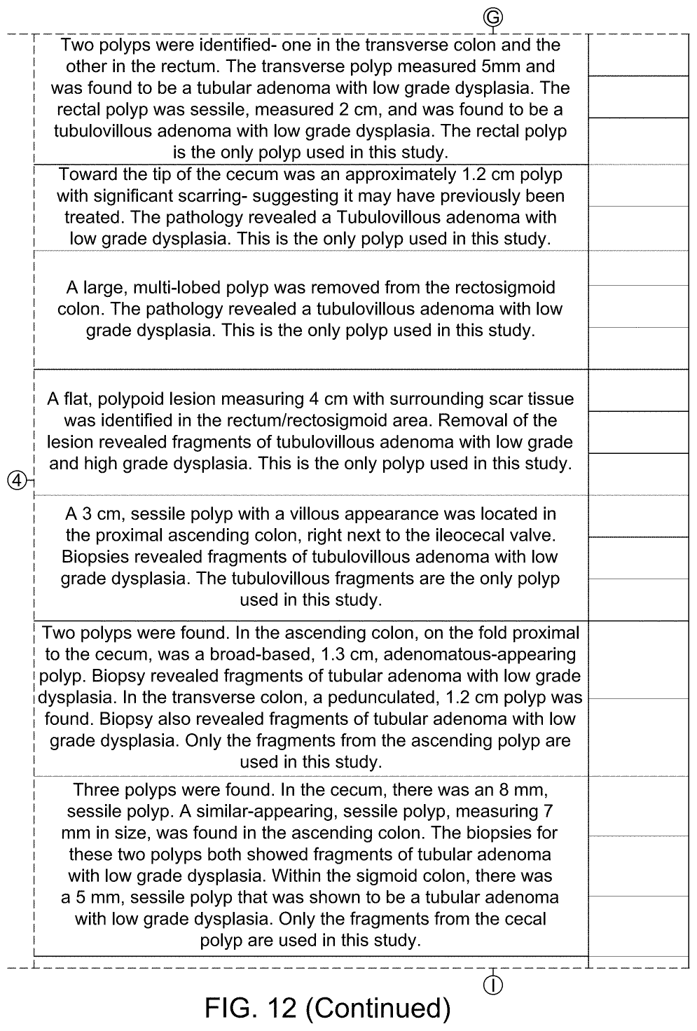

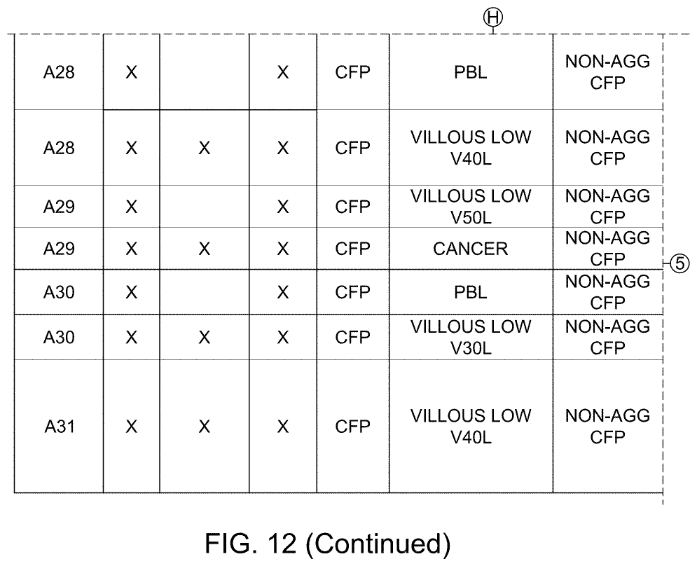

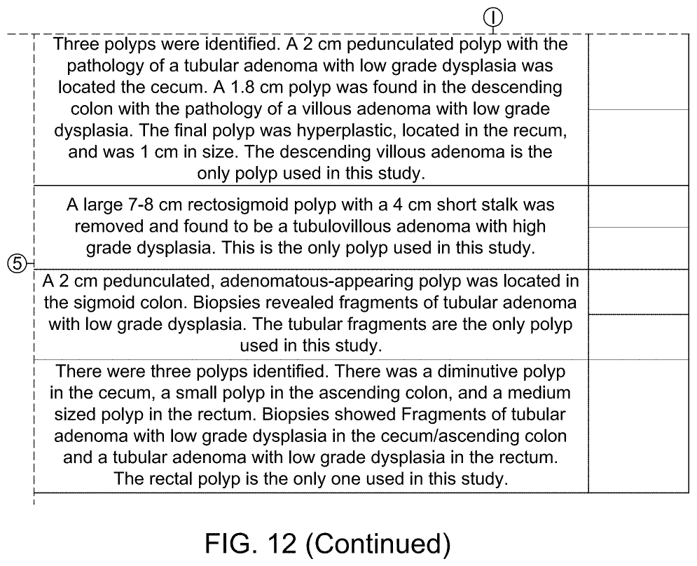

A model of the adenoma to carcinoma transition in a human tissue cases that were classified as Cancer Adjacent Polyp (CAP) and Cancer Free Polyp (CFP) patients was employed. The CAP cases capture the peripheral blood leukocytes (PBL) and/or normal colon epithelium, premalignant adenoma and the cancer tissue adjacent to the polyp (FIG. 1A). The CFP cases include the PBL and/or normal colon epithelium, and the premalignant adenoma that is not associated with cancer (FIG. 1B). The CAP and CFP polyp tissues were indistinguishable based on the polyp's size, histology and degree of dysplasia. Whole Genome Sequencing, RNA-sequencing, and methylation analysis (by Reduced Representation Bisulfite Sequencing) were performed on 16 CAP and 15 CFP cases, which included multiple tissues per case. This included 90 tissues by WGS, 69 by RNA-seq, and 76 by RRBS (Table 1; FIG. 12).

### Whole Genome Sequencing (WGS) Analysis

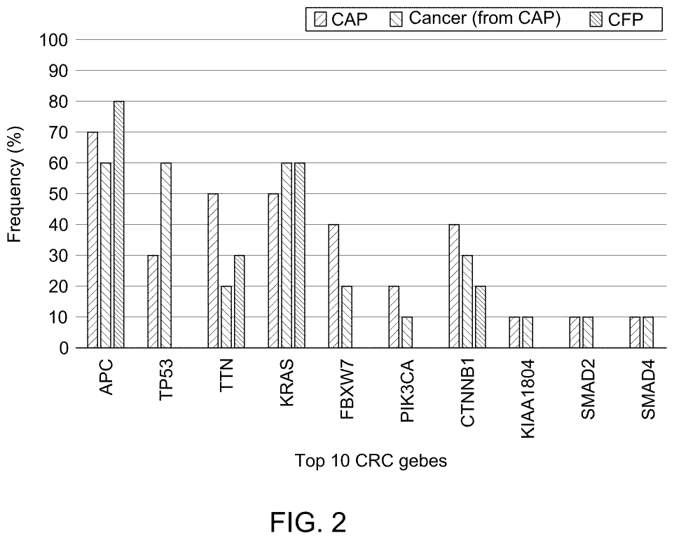

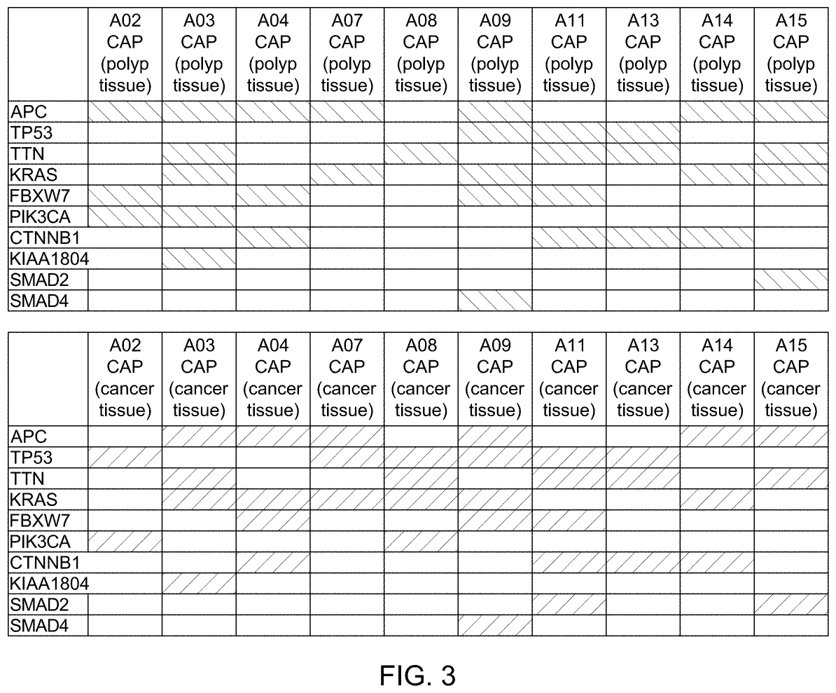

Genes with single nucleotide variants (SNVs) were determined that were distinct between CAP and CFP tissues by k-nearest neighbors algorithm (FIG. 1C). SNVs in APC were found at a high frequency in the CFP and CAP tissues (70% and 80%, respectively, FIG. 1D) as well as the CRC tissue (60%, FIG. 2). This was also the case for KRAS and BRAF. There were 38 genes with SNVs that were uniquely found in the CAP and adjacent CRC tissue, but not in the CFP tissue, including TP53 (FIGS. 1C and 1D; FIG. 2). There was only one gene, MUC19, which was unique to CFPs and was not found in CAPs or CRC tissues. For CAPs, in the majority of the genes and patients the mutation was first observed in the polyp tissue and persisted in the matched cancer tissues (FIG. 3). The cancer tissues tended to acquire mutations in these genes even if they weren't first observed in the polyp tissue. The exceptions of APC (in A02) and FBXW7 (in A02) were observed in the polyp tissue, but the not corresponding CRC tissue.

The Cancer Genome Atlas (TCGA) Network performed a study that identified consistently mutated somatic genes in non-hypermutated CRC (Cancer Genome Atlas, Nature 487:330-337 (2012)). The 10 most frequently mutated genes were APC, TP53, TTN, KRAS, FBXW7, PIK3CA, CTNNB1, KIAA1804, SMAD2, and SMAD4. The somatic mutation frequency of these 10 genes between the CAP and CFP tissues was compared and found that with the exception of APC and KRAS, the CAPs exhibited a higher frequency of mutations than the CFPs (FIG. 1D). For TP53, FBXW7, PIK3CA, KIAA1804, SMAD2 and SMAD4 the mutations were exclusively in CAP patients.

The most significantly mutated genes for CAPs, corresponding CAP cancer, and CFPs (as compared to either PBL or normal) were determined using the MutSig algorithm as described elsewhere (see, e.g., Lawrence et al., Nature 499:214-218 (2013)). A heatmap was drawn based on the Spearman's rank correlation of significantly mutated genes between each group (e.g., between CAP and normal, etc.). The mutation significance for each gene was identified by MutSig according to the mutation profiles of samples from the same group. The genes were then ranked by the p-value reported by MutSig and only genes with p-value <0.05 were involved in the Spearman's rank correlation calculation (FIG. 1E). It was clear from the heatmap that the CFP-normal or CFP-PBL comparison is the least correlated with the CAPs or the corresponding CAP cancer tissues. When comparing Pearson correlations between cases on the basis of their SNVs, the CFP tissues have a very low, negative correlation with CAPs or cancer tissues (r=−0.23 and −0.26, respectively). The CAPs and cancer tissues have a high correlation (r=0.79; Table 2).

TABLE 2
Pearson correlations of SNVs between each tissue
type: CFP polyp, CAP polyp, CAP cancer.

| | CFP | CAP | Cancer |
|---|---|---|---|
| CFP | 1 | −0.226556 | −0.255643 |
| CAP | −0.226556 | 1 | 0.7908353 |
| Cancer | −0.255643 | 0.7908353 | 1 |

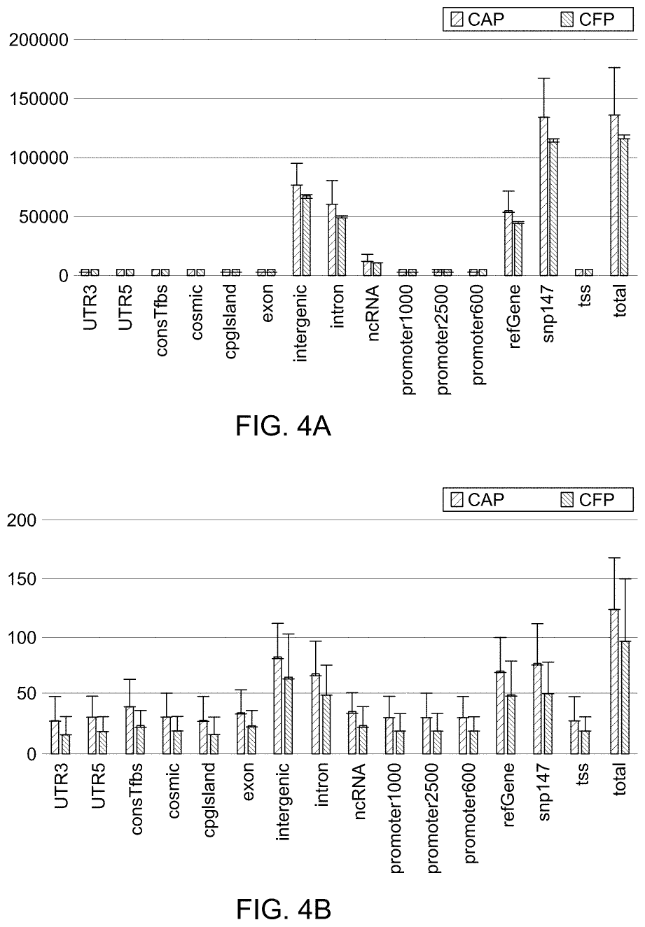

The mean distribution of somatic single nucleotide variants (SNV), INDELs, copy number variation (CNV) and structural variants (SVs) between the CAP and CFP tissues was next examined. There were overall more SNVs called for the CAPs than the CFPs (vs. Normal: p=0.03 Paired t-test on mean, vs. PBL: p=0.02; pooled: p=0.03; FIG. 1F). SNVs between individuals with CFPs were more homogeneous, meaning less heterogeneity of the SNVs than in the CAP tissues (vs. Normal: p=0.03 Paired t-test on stdev; vs. PBL: p=0.02; pooled: p=0.02). There were also more somatic INDELS (pooled: p=0.02) and SVs (pooled: p=2.39×10⁻⁸) with more heterogeneity in the CAPs as compared to CFPs (FIG. 4). Analysis using Kyoto Encyclopedia of Genes and Genomes (KEGG) of the somatic mutations that differed between the CAPs and CFPs indicated enrichment for genes in “Pathways in cancer” (58/397 genes, p=0.0001) among others (Table 3; FIG. 13).

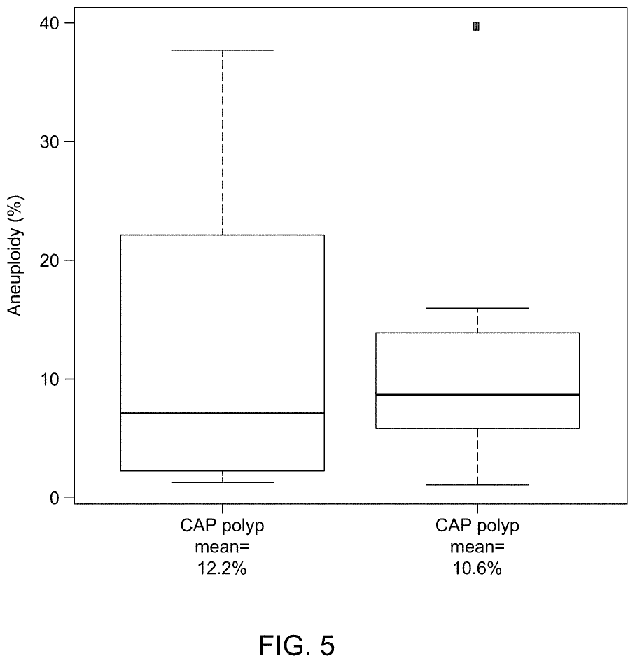

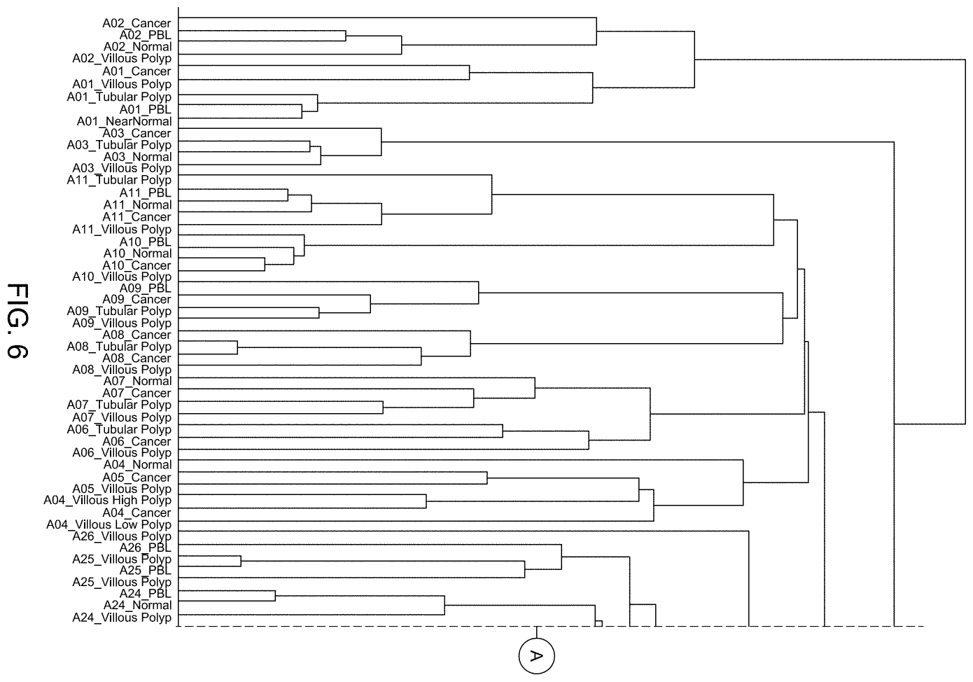

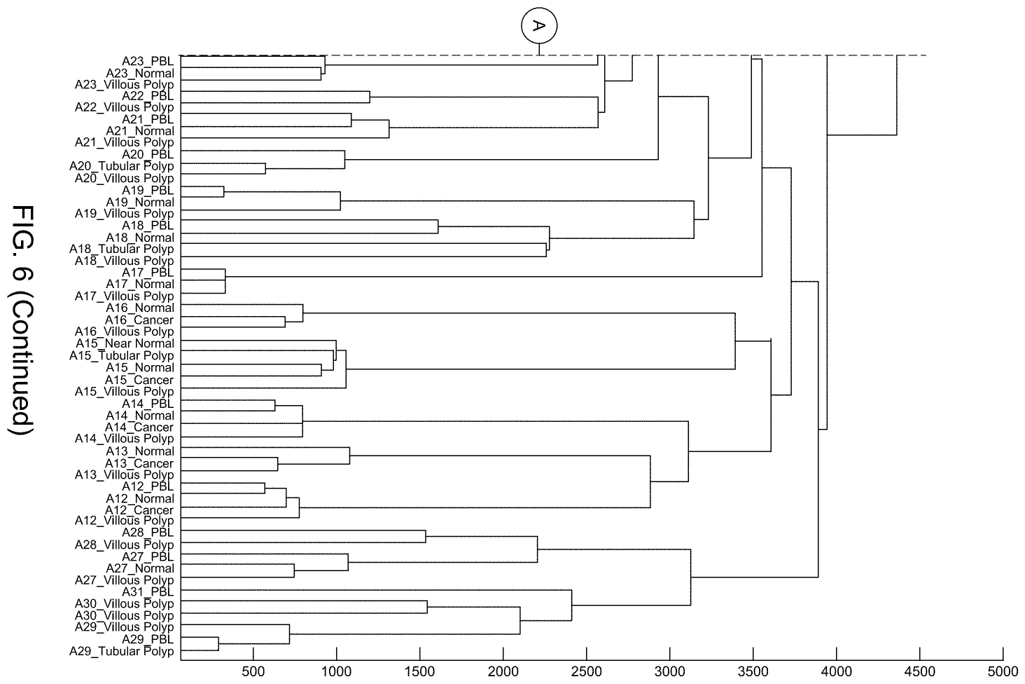

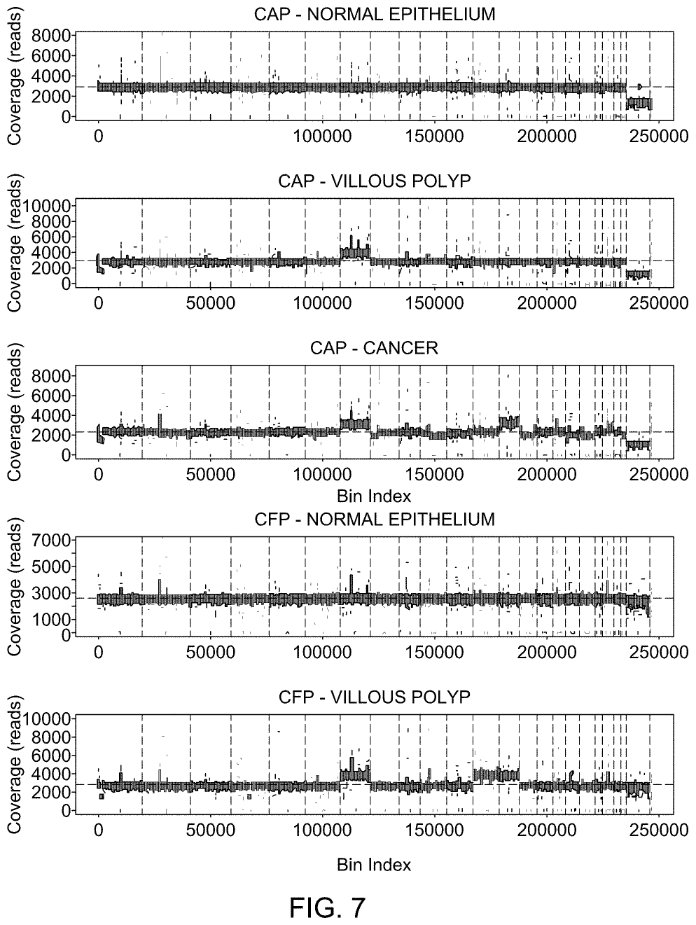

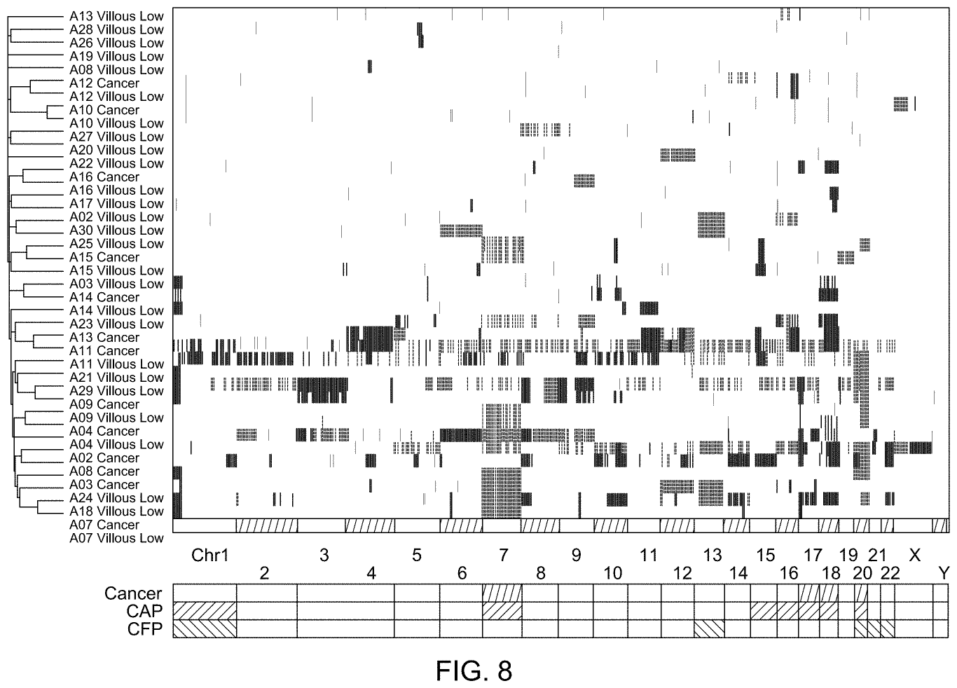

The CAPs showed a higher amount of CNV and percentage of aneuploidy than CFPs (FIG. 5). The CNV in each tissue compartment from the same patient tended to cluster together, and most CNVs were shared from different tissues of the same patient even with common CNVs across all patients/samples removed (FIG. 6). There was large scale aneuploidy in both the CAP and CFP cases, beginning in the polyp compartment (FIG. 7). The aneuploidy observed in the CAP had both overlap with the cancer compartment as well as unique regions of aneuploidy. There were both specific and unique regions of CNV on a per-chromosome basis for CAPs, corresponding cancer, and CFPs (FIG. 8). To compare regions of CNV on a per-chromosome basis, a pairwise similarity metric was utilized that characterizes duplications or deletions on a chromosome that is present in both samples. The similarity metric produces a score between 0 and 1 for each chromosome and a higher score indicates that more samples had overlapping CNV. This analysis identified chromosomes with more CNVs compared to other chromosomes for each CAP, cancer and CFP tissue type. Chromosomes 1, 7, 15, 16, 17, 18 and 20 had the most recurrent CNV across CAPs, chromosomes 7, 17, 18, and 20 across cancers, and chromosomes 1, 13, 20, 21, and 22 were most recurrent across CFPs.

### RNA-Seq Analysis

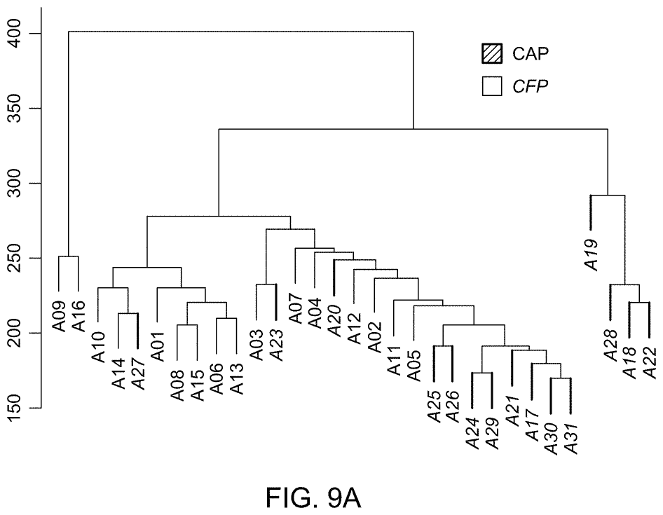

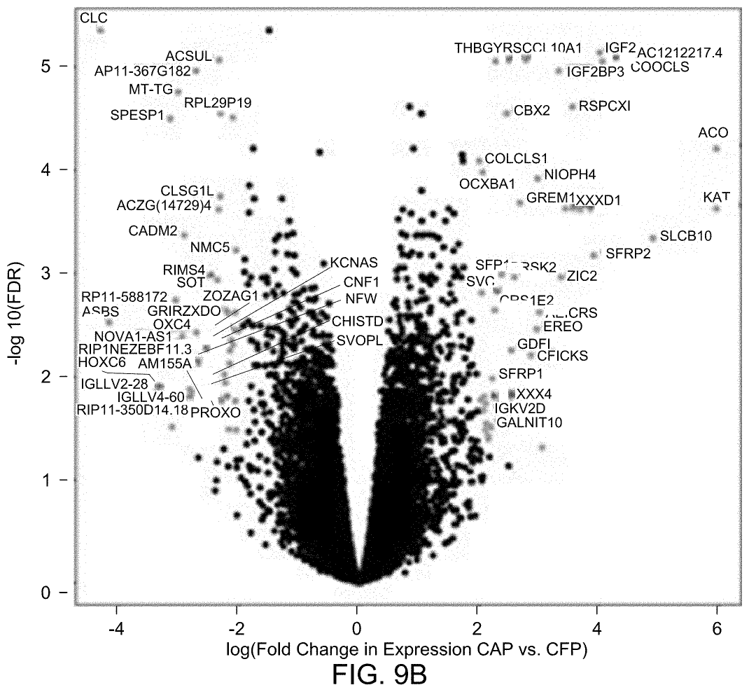

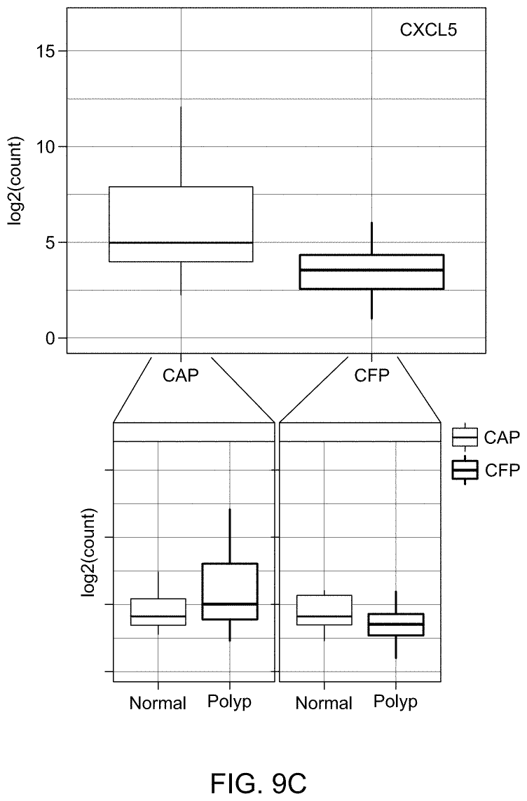

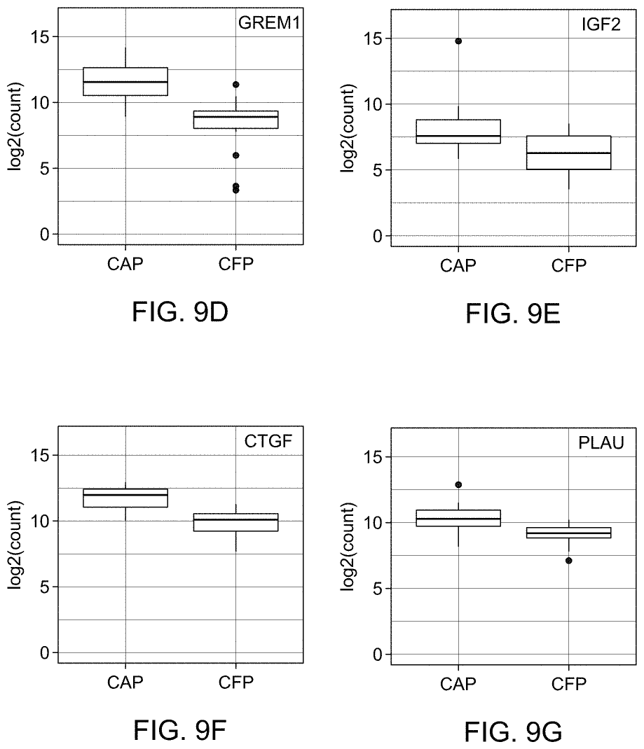

The analysis of genes differentially expressed between the CAP and CFP tissues identified 2,452 genes that were significantly different between the groups (Table 4; FIG. 14). When all the cases were clustered based on their average distance, where samples that are most similar occupy closer locations on the dendrogram, the majority of the CAPs and CFPs cluster distinctly from one another (FIG. 9A). Since it was observed from the WGS data that the mutational profiles of CAPs correlated higher with the cancer tissue than did the CFPs, if this relationship was similar for RNA-seq data was determined. The expression of CAPs and CFPs to the cancer tissue was compared and it was found that there are fewer genes with differential expression above the FDR and fold change cut-off when the CAP and cancer tissues are compared than when the CFP and cancer tissues are compared indicating the CAPs are more similar to the corresponding cancer tissue than are the CFPs (Table 5).

TABLE 5
Number of genes with differential expression based on FDR and
fold change between CAPs and cancer and CFPs and cancer tissue.

| | # of genes with | # of genes with |
|---|---|---|
| | FDR < 0.1 | fold change > 2 |
| CFP vs cancer | 8370 | 640 |
| CAP vs cancer | 3135 | 258 |

Specific genes of interest that were differentially expressed between CAPs and CFPs by plotting those with the lowest False Discovery Rate (FDR) (<0.1) and highest fold change (higher than 2, in either direction) (Table 6; FIG. 15) were next identified. This represented ˜100 genes, and there was no trend in whether there was increased or decreased expression changes overall between CAPs and CFPs (FIG. 9B). This enabled examination of genes on an individual basis, and many genes important in the development of CRC, and cancer in general, were upregulated in the CAP tissues relative to the CFPs, including CXCL5 (FIG. 9C), GREM1 (FIG. 9D), IGF2 (FIG. 9E), CTGF (FIG. 9F) and PLAU (FIG. 9G).

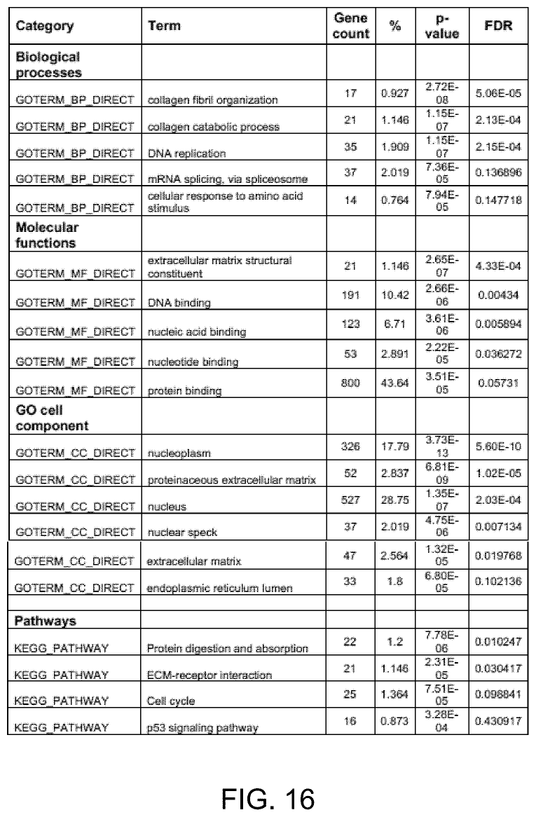

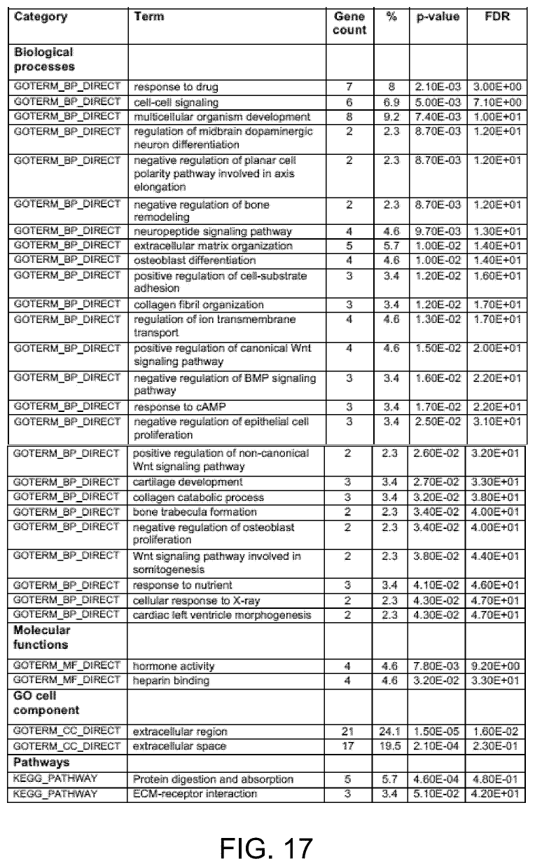

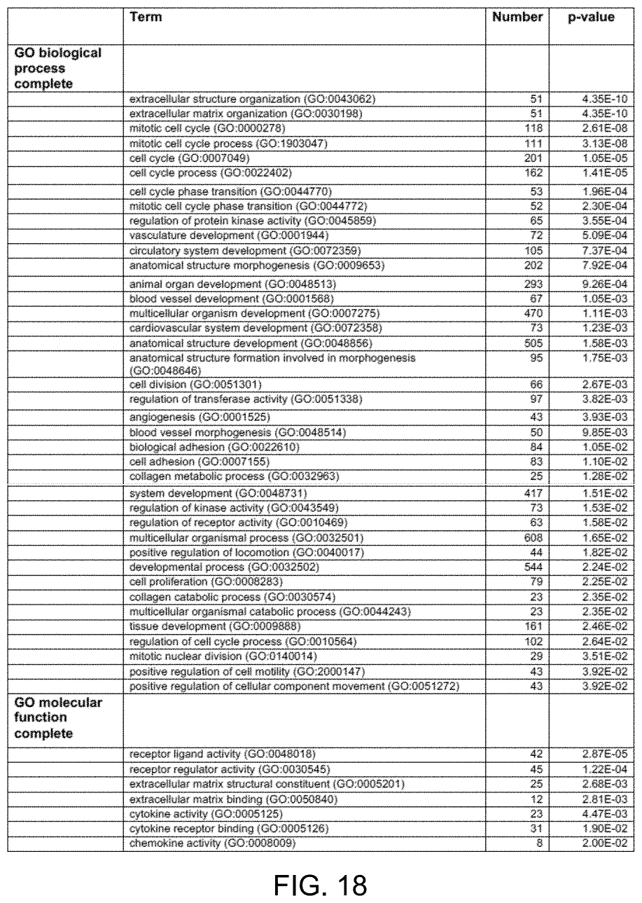

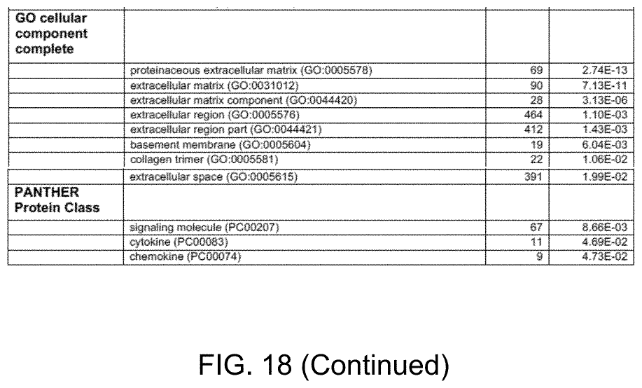

Enrichment analysis of the 2,452 differentially expressed genes and the 102 genes with the lowest FDR and highest fold change between CAPs and CFPs was performed using DAVID (Tables 7 and 8; FIGS. 16 and 17) as described elsewhere (see, e.g., Huang et al., Nat. Protoc. 4:44-57 (2009); Huang et al., Nucleic Acids Res. 37:1-13 (2009); and Mi et al., Nucleic Acids Res. 45:D183-D189 (2017)). The gene ontology of biological processes, molecular functions, and cell component were analyzed as well as pathway analysis by KEGG for both gene sets. The 2,452 differentially expressed genes were enriched in the KEGG pathways involved in protein digestion and absorption (1.2%, p=7.8×10⁻⁶), ECM-receptor interaction (1.1%, p=2.3×10⁻⁵), cell cycle (1.4%, p=7.5×10⁻⁵) and p53 signaling pathway (0.87%, p=3.3×10⁻⁵). The 102 genes with lowest FDR and highest fold change were also enriched in the KEGG pathways involved in protein digestion and absorption (5.7%, p=4.6×10⁻⁴) and ECM-receptor interaction (3.4%, p=5.1×10⁻²). In PANTHER a list of gene-value pairs was analyzed which was the gene and corresponding fold change of 2,452 genes that were significantly differentially expressed between CAPs and CFPs, and also found enrichment for extracellular matrix organization (51, p=4.31×10⁻¹⁰) and cell cycle (201, p=1.05×10⁻⁵) (Table 9; FIG. 18).

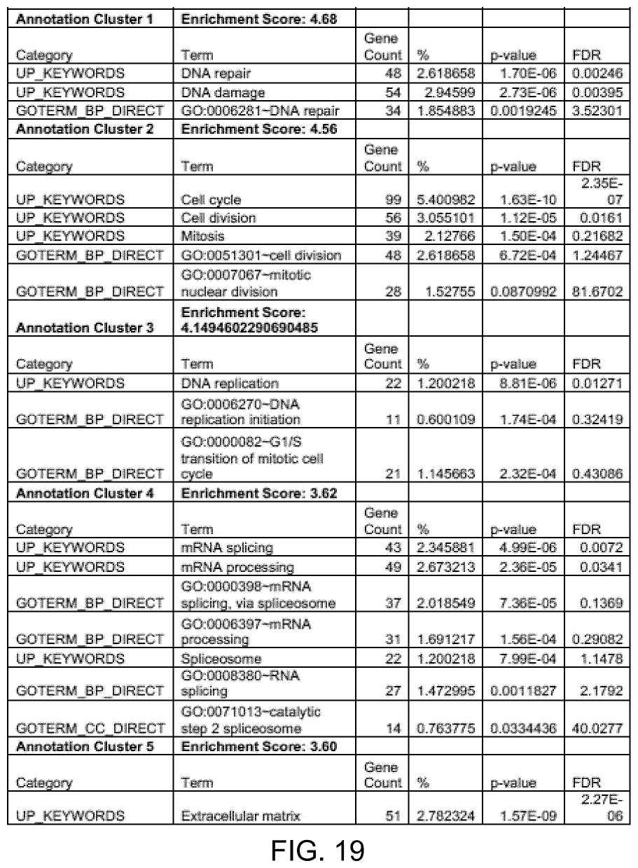

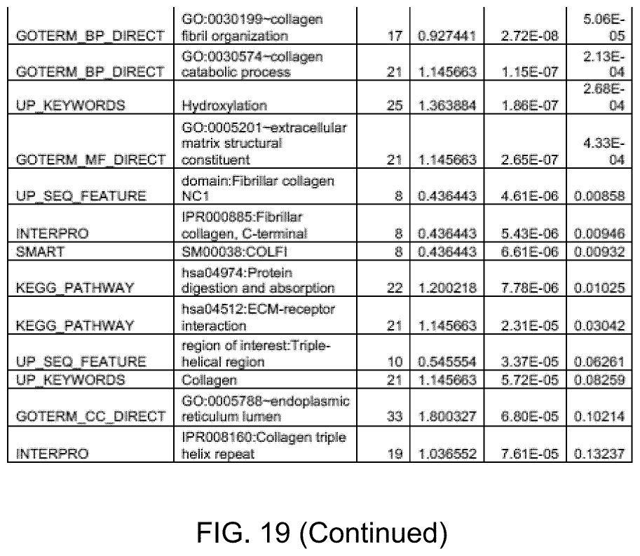

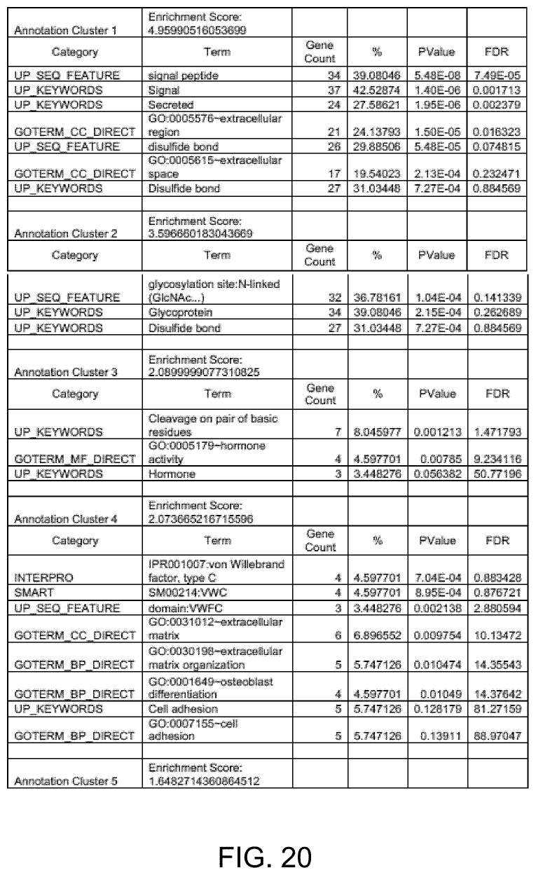

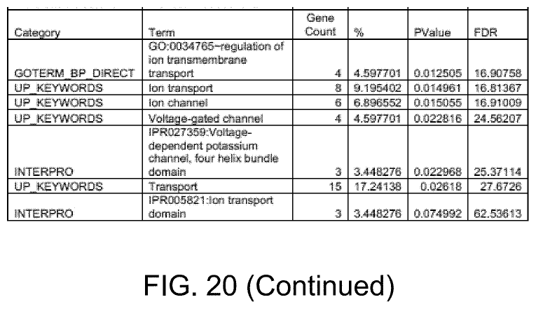

The top 5 functional annotation clusters for the 2,452 differentially expressed genes and the 102 genes with the lowest FDR and highest fold change between CAPs and CFPs (Tables 10 and 11; FIGS. 19 and 20) were also analyzed. The functional annotation clusters using the 2,452 differentially expressed genes consisted of gene ontology biological processes of DNA repair (1.8%, p=0.0019), cell division (2.6%, p=6.7×10⁻⁴), DNA replication initiation (0.6%, p=1.7×10⁻⁴), mRNA splicing via spliceosome (2.0%, p=7.3×10⁻⁵), and collagen fibril organization (0.9%, p=2.7×10⁻⁸). The functional annotation clusters using the 102 genes with lowest FDR and highest fold change consisted of signal peptide (39%, p=5.5×10⁻⁸), extracellular region (24%, p=1.5×10⁻⁵), and glycosylation (37%, p=1.0×10⁻⁴).

### RRBS Analysis

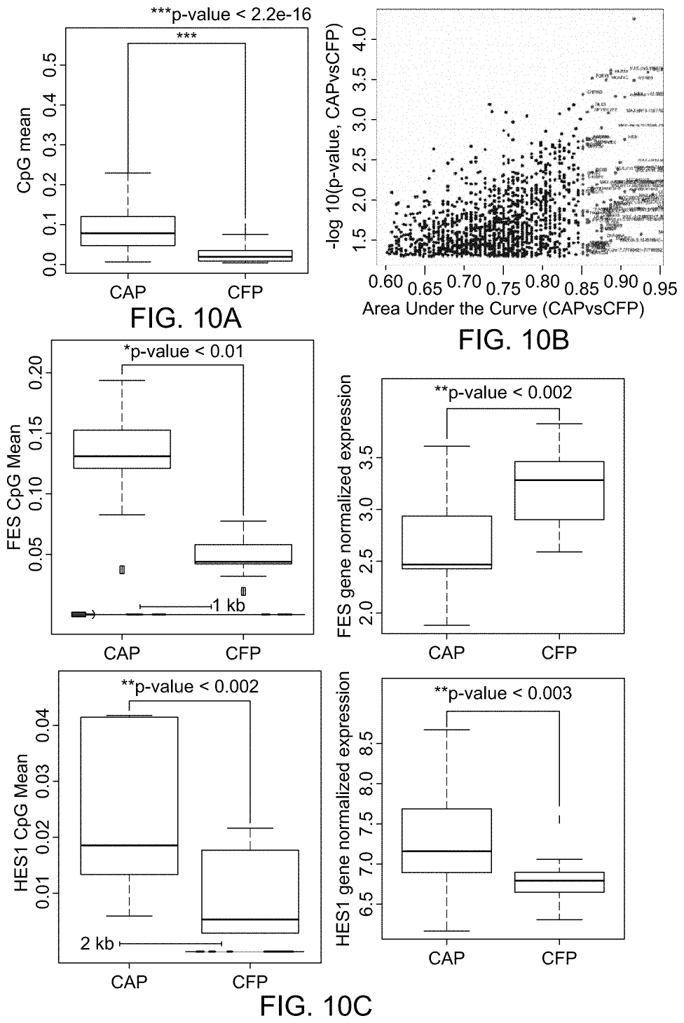

Differentially methylated regions (DMRs) were calculated based specifically on hypermethylation between CAP and CFP tissues, and increased methylation of DMRs was found in the CAPs (p<2.2e-16; FIG. 10A). Both the fold change (>20) and area under the curve (>0.85) were examined for the significant (p<0.05) DMRs between the CAP and CFP tissues, and found 30 and 87 genes with increased methylation of DMRs above these thresholds, respectively (FIG. 10B; Tables 12 and 13; FIGS. 21 and 22). The relationship between gene expression and hypermethylation in some cases directly correlated but in others there was an inverse correlation. For example, FES has an increase in promoter hypermethylation (FC=4.5, p=0.01) and lower FES gene expression (FC=−0.51, p=0.03) in the CAPs as compared to the CFPs (FIG. 10C). Conversely, HES1 has both an increase in hypermethylation (FC=2.5, p=0.001) and higher gene expression (FC=0.59, p=0.04) in the CAP tissues compared to the CFP tissues (FIG. 10D).

Integration of Results from Genome, Transcriptome and Methylome Analyses

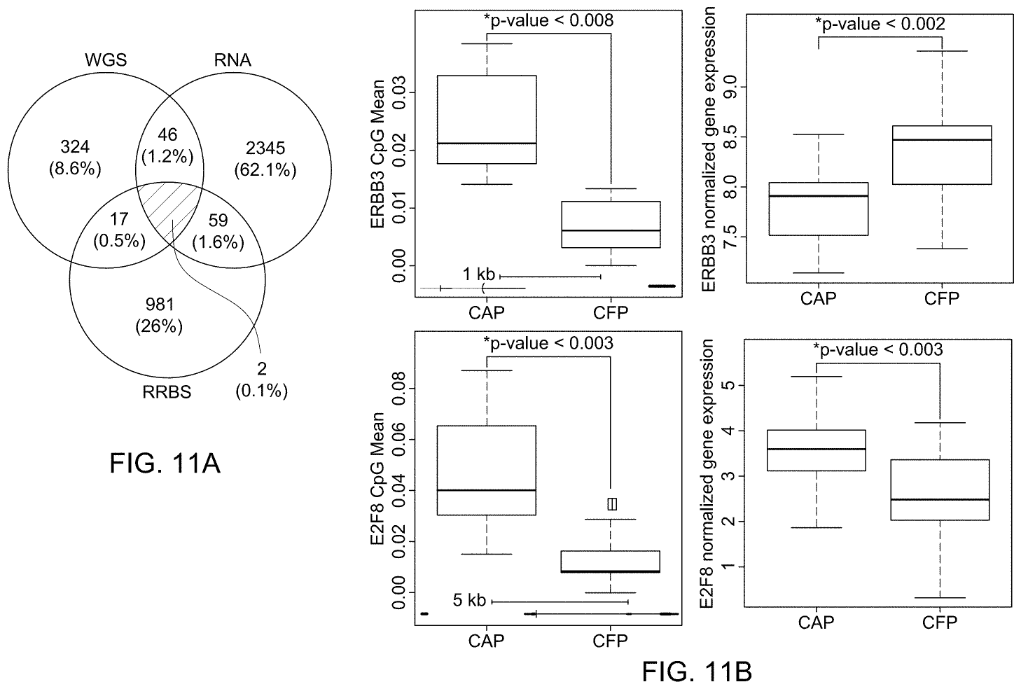

The overlap of alterations discovered between the CAP and CFPs across the sequencing platforms that we performed, and identified 2 genes which were differentially altered between the CAPs and CFPs across the three platforms studied was characterized. ERBB3 and E2F8 each had a genetic variant, differential expression and differentially methylated regions (FIG. 11A). Additionally, there was overlap between all pairwise comparisons, which resulted in a panel of 124 genes that have at least two alterations (genetic variant and expression change, genetic variant and methylation change, expression and methylation change, or all three; Table 14). There were two genes with overlap in all platforms, ERBB3 and E2F8. ERBB3 had high methylation (Fold Change (FC)=3.3, p=0.008) and lower expression (FC=−0.55, p=0.04) in the CAP compared to the CFP tissues (FIG. 11B). E2F8 had both high methylation (FC=4.3, p=0.03) and expression (FC=0.95, p=0.04) in the CAP compared to the CFP tissues (FIG. 11C).

TABLE 14
124 gene panel in common for all pairwise comparisons of WGS,
RNA-seq, and RRBS differential between CAP and CFP polyps

| | | | | | |
|---|---|---|---|---|---|
| E2F8 | COL2A1 | GREM1 | COL6A3 | SCARF2 | STK33 |
| ERBB3 | P2RY6 | IGSF22 | CNTN4 | EFNB3 | ZNF579 |
| NEB | HES1 | STX8 | NUP210 | MEGF10 | GPC1 |
| KIAA0825 | GRIN2C | BRSK2 | ARIH2 | SATB1 | SCN5A |
| PPARG | RARG | SOCS3 | HHIP | RGMA | ANKRD36 |
| NPC1L1 | TNNC2 | PRKACB | MED7 | ZNF141 | ALPPL2 |
| TRRAP | TK1 | C11orf63 | RIMS2 | BCL2L10 | C4orf33 |
| GYLTL1B | C1orf86 | ZNF480 | TAF1L | GBGT1 | SST |
| FBN1 | EBF4 | NPW | TNC | FGF18 | COG6 |
| NOX5 | ZNF470 | PLXDC1 | ATHL1 | SNCAIP | IGF2 |
| KMT2B | CRYBA2 | IL11 | CD248 | NACAD | ACSL6 |
| A1BG | CABP7 | THRB | NUAK1 | MATK | FARP1 |
| CACNA1I | TRPC1 | LYL1 | RPH3A | KCNN2 | CLYBL |
| SLITRK2 | AHSA2 | CHRD | CIT | DPY19L2P2 | IGDCC3 |
| COL12A1 | HEBP1 | COL4A3 | ISLR | DNAH9 | CDH3 |
| ST6GALNAC5 | ZNF599 | GPRIN2 | TANC2 | SPEG | RASAL3 |
| HMCN1 | TRPV3 | CR2 | OTOP3 | COL13A1 | CPLX1 |
| DUSP2 | MT1JP | NOTCH3 | ZNF726 | ROBO3 | CCK |
| SLC5A7 | TSPY26P | FADS1 | PLEKHG2 | CACNA1H | LILRA1 |
| COL5A2 | ZNF836 | FES | RIMS1 | VANGL2 | MUC4 |
| BAIAP3 | PLEKHH2 | GPR98 | COL11A2 | | |

### Methods

### Patient Sample Characteristics and Tissue Preparation.

All tissues were collected at Mayo Clinic between 2000-2016 through an IR approved Biobank for Gastrointestinal Health Research [BGHR] (IRB 622-00, PI LA Boardman). Informed consent through this IRB was obtained from all participants in this study, and all methods were carried out in accordance with all guidelines and regulations outlined within this IRB. Polyp tissues with adjacent tumor and normal colonic epithelium full thickness specimens at least 8 cm from the polyp/tumor margin were harvested following surgical resection and snap frozen in liquid nitrogen and maintained in a −80 freezer. Cancer free polyps and normal colonic epithelium at least 8 cm from the polyp were collected at the time of colonoscopic resection. Cancer adjacent polyps (CAPs) were matched to the cancer free polyps (CFPs) based on polyp size (categorical size: 1 to 2 cm, 2-5 cm and >5 cm); histology (villous features) and degree of dysplasia. All polyps presented in this study were adenomatous polyps with villous features (tubulovillous or villous), and with low-grade dysplasia only. All CAP and CFP cases exclude subjects with a prior history of any malignancy; a family history of Lynch syndrome or FAP; any other syndrome associated with hereditary CRC or inflammatory bowel disease. All tissue used in this study was removed prior to neoadjuvant/adjuvant therapy with the exception of one case (A04), which was collected after neoadjuvant treatment (FOLFOX) for Stage IV, metastatic colorectal adenocarcinoma. Peripheral blood leukocytes from the patients were obtained when possible prior to removal of the tissue, and any neo-adjuvant/adjuvant treatment.

Tissues were macro-dissected using a hematoxylin and eosin (H&E) guide that was used to mark areas of normal epithelium, polyp or cancer by a pathologist. DNA was extracted with the PureGene method, and RNA was extracted using Qiagen MiRNeasy mini kit. Nucleic acids were quantified with appropriate kits on the Qubit Fluorometer.

### Whole Genome Sequencing (WGS), RNA-Seq and Reduced Representation Bisulfite Sequencing (RRBS) Processing and Analyses

All samples were subjected to WGS on the Illumina HiSeq X instruments producing 150 base pair, paired-end reads to meet a goal of 30× mean coverage at the Broad Institute, RNA-seq using the Illumina TruSeq™ Stranded mRNA Sample Preparation kit on the Illumina HiSeq 2000, or HiSeq 2500 producing 101 base pair paired-end reads at the Broad Institute, and RRBS using the TruSeq SBS sequencing kit version 3 on the Illumina HiSeq 2000 producing 51 base pair paired-end reads at the Mayo Clinic.

WGS data was processed using the Picard Informatics Pipeline, with all data from a particular sample aggregated into a single BAM file which included all reads, all bases from all reads, and original/vendor-assigned quality scores. A pooled Variant Call Format (VCF) file using the latest version of Picard GATK software was generated and provided for each sample batch. Data for RNA-seq was analyzed using the Broad Picard Pipeline, which includes de-multiplexing and data aggregation. RRBS Data was collected using HiSeq data collection version 1.5.15.1 software, and the bases were called using Illumina's RTA version 1.13.48.

For library construction, total DNA was quantified in triplicate using the QuantiT™ PicoGreenR DNA Assay Kit and normalized to 2 ng/L minimum concentration. An aliquot of 100 ng for each sample was transferred into library preparation utilizing the Broad Institute developed one-well protocol. All biochemistry occurs in a single well without the need for sample transfer (the sample was reversibly immobilized to and released from magnetic beads, allowing washes and reagent addition). The one-well protocol streamlines the process and greatly reduces sample input requirements. The product provides one library (typical median insert size of library is 330 bp; see, e.g., Fisher et al., Genome Biol. 12:R1 (2011)). Details on the library preparation workflow including general information on the adapters can be found at, provided by Illumina:

www.illumina.com/content/dam/illuminamarketing/documents/products/datasheets/datasheet_truseq_sampleprep_kits.pdf.

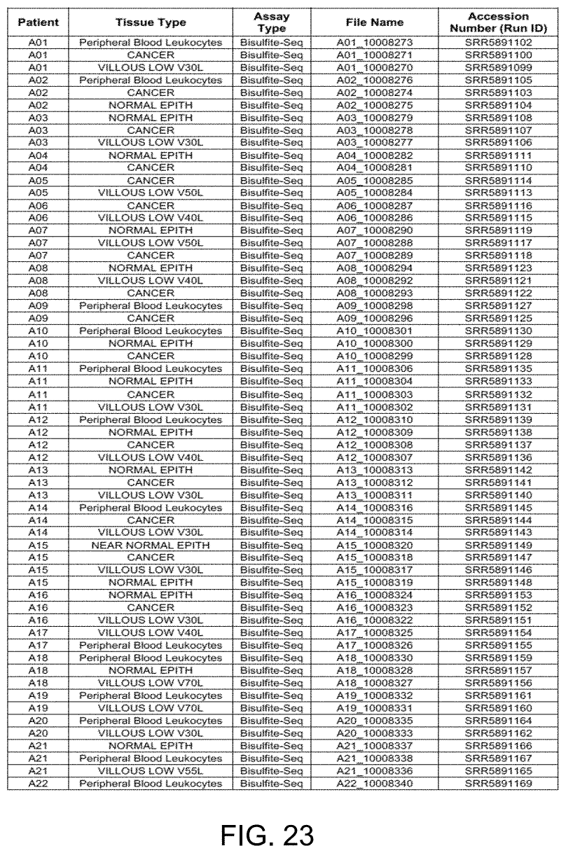

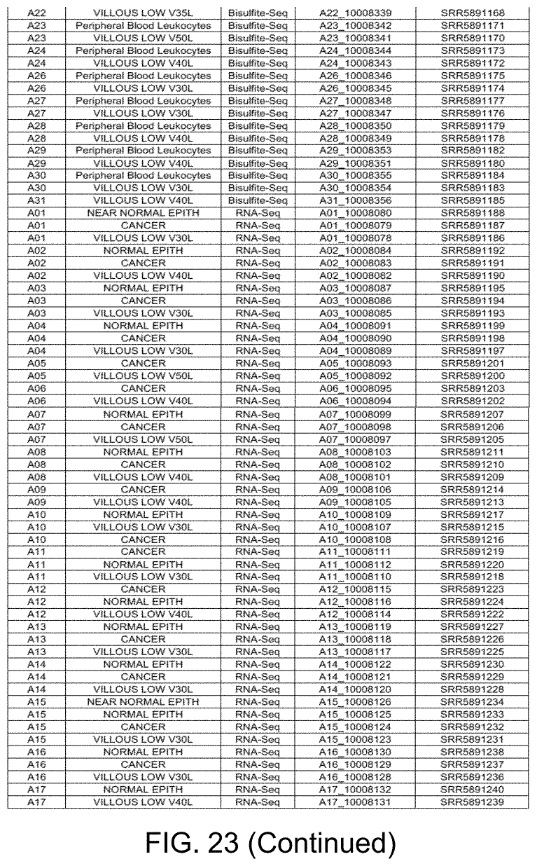

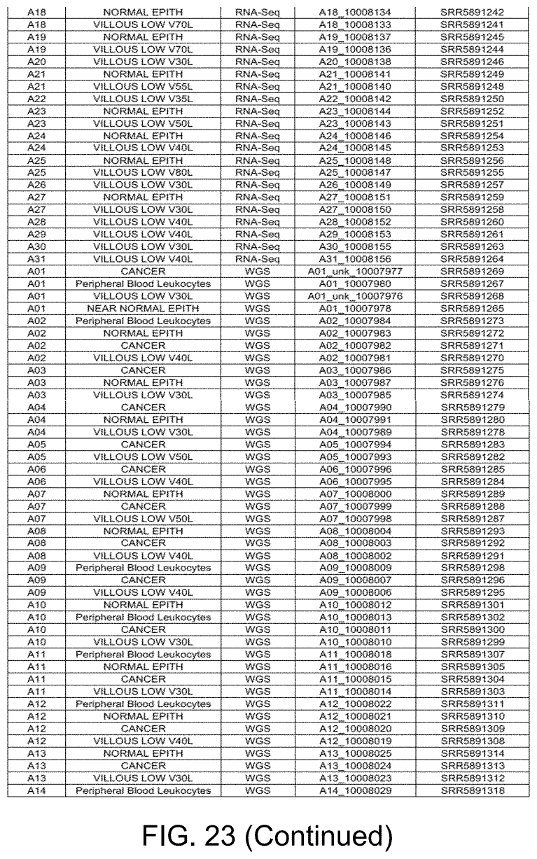

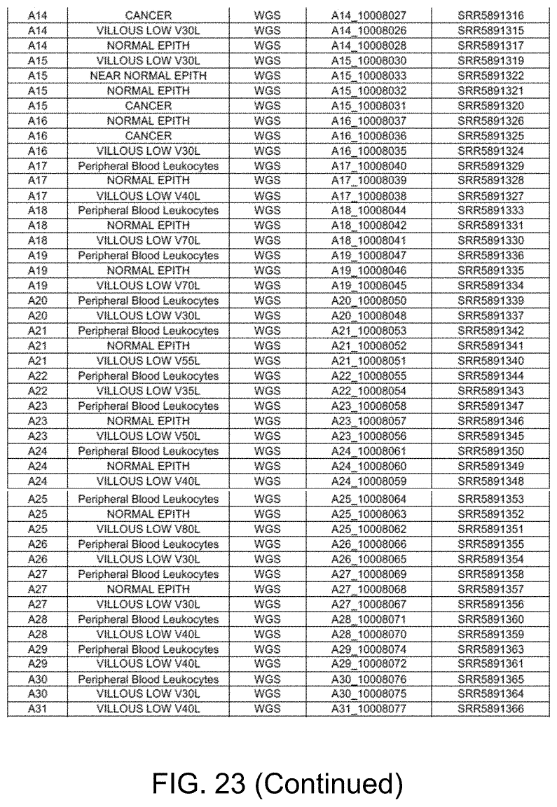

Samples were sequenced on the Illumina HiSeq X instruments producing 150 base pair, paired-end reads to meet a goal of 30× mean coverage. Using the Picard Informatics Pipeline, all data from a particular sample was aggregated into a single BAM file which included all reads, all bases from all reads, and original/vendor-assigned quality scores. A pooled Variant Call Format (VCF) file using the latest version of Picard GATK software was generated and provided for each sample batch. All whole genome sequencing data analyzed in this manuscript are available in the dbGaP database with Study Accession number: phs001384.v1.p1. Accession numbers for each WGS BAM file are located in Table 15 (FIG. 23).

### Genomic Alteration Detection

Before calling germline and somatic mutations, we followed GATK's best practice to preprocess the data. Reads were first quality-controlled and then mapped to the reference. Duplicates were marked by Picard, and then GATK was used for later analyses, including base recalibration and variant calling. CNVs were called by CNVnator as described elsewhere (see, e.g., Abyzov et al., Genome Res. 21:974-984 (2011)). In order to detect somatic single nucleotide variants (SNVs) between the polyp or tumor and matched normal tissue or PBL, 4 different somatic variant callers were used: MuTect2, SomaticSniper, Strelka, and VarScan (see, e.g., Cibulskis et al., Nat. Biotechnol. 31:213-219 (2013); Koboldt et al., Genome Res. 22:568-576 (2012); Larson et al., Bioinformatics 28:311-317 (2012); and Saunders et al., Bioinformatics 28:1811-1817 (2012)). Those callers were run with default options for normal and polyp or tumor samples from each patient. Common SNVs detected by at least 2 different callers were included. Variant allele frequencies for those SNVs were calculated from sample BAM files for each patient using an in-house script. To annotate mutations, Variant Effect Predictor (www.ensembl.org/Tools/VEP) and ANNOVAR (see, e.g., Wang et al., Nucleic Acids Res. 38:e164 (2010)) were used.

### RNA-Seq and Processing

Total RNA was quantified using the Quant-iT™ RiboGreenR RNA Assay Kit and normalized to 5 ng/μl. 200 ng of RNA was used to prepare libraries, using an automated version of the. mRNA was selected from the total RNA samples using oligo dT beads. The cDNA that resulted was indexed using Broad Institute designed indexed adapters substituted in for multiplexing. After enrichment the libraries were quantified with qPCR using the KAPA Library Quantification Kit for Illumina Sequencing Platforms and then pooled equimolarly. Each sequencing run was 101 bp paired-end with barcoding. Pooled libraries were normalized and denatured prior to sequencing. Flow cell cluster amplification and sequencing were performed according to the manufacturer's protocols using either the HiSeq 2000 or HiSeq 2500. Data was analyzed using the Broad Picard Pipeline, which includes de-multiplexing and data aggregation.

FASTQ files were converted from BAM files using Broad's Picard software (available online at broadinstitute.github.io/picard/). The FASTQ files were analyzed using Mayo Clinic's standard RNA-Seq application, MAP-RSeq v.2.0.0 (available online at bioinformaticstools.mayo.edu/research/maprseq/). MAP-RSeq is an integration of open source bioinformatics tools along with in-house developed methods to process and analyze paired-end RNA-Seq data. Read alignment was performed with Tophat as described elsewhere (see, e.g., Trapnell et al., Bioinformatics 25:1105-1111 (2009)), using Bowtie as described elsewhere (see, e.g., Langmead et al., Genome Biol. 10:R25 (2009)). Reads were aligned to the transcriptome (Ensembl GTF) and genome (hg19), and expression was quantified using featureCounts as described elsewhere (see, e.g., Liao et al., Bioinformatics 30:923-930 (2014)). RPKM values were calculated from the raw gene counts to assess the relative abundance of each gene. Within each sample, RSeQC software was used to detect unsymmetrical gene body coverage, high levels of read duplication, and low saturation levels of known exon junctions as described elsewhere (see, e.g., Wang et al., Bioinformatics 28:2184-2185 (2012)). Reads were additionally normalized using conditional quantile normalization, which adjusts for gene length, GC content and library size as described elsewhere (see, e.g., Hansen et al., Biostatistics 13:204-216 (2012)). All RNAseq data analyzed in this manuscript are available in the dbGaP database with Study Accession number: phs001384.v1.p1. Accession numbers for each RNA-seq BAM file are located in Table 15 (FIG. 23).

### Reduced Representation Bisulfite Sequencing (RRBS) and Processing

RRBS was performed at the Mayo Clinic Genotyping Shared Resource facility. Briefly, DNA (250 ng) was digested with Msp1 (New England Biolabs, Catalog Number: R0106M) and purified using Qiaquick Nucleotide Removal Kit (Qiagen, Catalog Number: 28004). End-repair A tailing was performed (New England Biolabs, Catalog Numbers: M0212L) and TruSeq methylated indexed adaptors (Illumina, Catalog Number: 15025064) were ligated with T4 DNA ligase (New England Biolabs, Catalog Number: M0202L). Size selection was performed with Agencourt AMPure XP beads (Beckman Coulter, Catalog Number: A63882). Bisulfite conversion was performed using EZ-DNA Methylation Kit (Zymo Research, Catalog Number: D5001) as recommended by the manufacturer with the exception that incubation was performed using 55 cycles of 95° C. for 30 seconds and 50° C. for 15 minutes. Following bisulfite treatment, the DNA was purified as directed and amplified using Pfu Turbo C Hotstart DNA Polymerase (Agilent Technologies, Catalog Number: 600414). Library quantification was performed using Qubits dsDNA HS Assay Kit (Life Technologies, Catalog Number: Q32854) and the Bioanalyzer DNA 1000 Kit (Agilent Technologies, Catalog Number: 5067-1504).

The final libraries from RRBS were prepared for sequencing per the manufacturer's instructions in the Illumina cBot and HiSeq Paired end cluster kit version 3. The samples were placed onto seven lanes of a paired-end flow cell at concentrations of 7-8 pM and the control sample, PhiX, was placed in the eighth lane to allow the sequencer to account for the unbalanced representation of cytosine bases. The flow cell was then loaded into the Illumina cBot for generation of cluster densities. After cluster generation, the flow cells were sequenced as 51×2 paired end reads using Illumina HiSeq 2000 with TruSeq SBS sequencing kit version 3. Data was collected using HiSeq data collection version 1.5.15.1 software, and the bases were called using Illumina's RTA version 1.13.48.

The RRBS data was processed using a streamlined analysis and annotation pipeline for reduced representation bisulfite sequencing, SAAP-RRBS (see, e.g., Sun et al., Bioinformatics 28:2180-2181 (2012)). Briefly, FASTQ were trimmed to remove adaptor sequences, and any reads with less than 15 bp were discarded. Trimmed Fastqs were then aligned against the reference genome using BSMAP as described elsewhere (see, e.g., Xi et al., BMC Bioinformatics 10:232 (2009)); this tool converts the reference genome to align the bisulfite treated reads. Samtools was used to get mpileup and custom PERL scripts to determine CpG methylation and bisulfite conversion ratios (see, e.g., Li et al., Bioinformatics 25:2078-2079 (2009)). Methylation was reported along with custom CpG annotation for the one with minimum of five read support. All RRBS data analyzed in this manuscript are available in the dbGaP database with Study Accession number: phs001384.v1.p1. Accession numbers for each RRBS BAM file are located in Table 15 (FIG. 23).

### Determining Differentially Methylated Regions

Tiled units of CpGs were created based on distance between adjacent CpG site locations (within 100 base pairs of the last observed CpG) and the level of background methylation in the control group (not to exceed 5%; control group were the CFPs). Regions of chromosomes satisfying these criteria with more than 5 CpGs were considered regions of interest. Each CpG was also be observed in at least 50% of the samples of each disease group to be considered. Statistical significance of these regions were determined by logistic regression using the ratio of methylated and total read counts within the region as a response and disease group as a covariate. To account for varying read depths across individual subjects, an over-dispersed logistic regression model was used, where dispersion parameter was estimated using the Pearson Chi-square statistic of the residuals from the fitted model.

### Statistical Analyses

Wilcoxon rank-sum test was used to test for differences between the two groups (CAP and CFP tissues). Unless specified in the Results or Figure Legend, analyses were performed as a comparison between the 16 CAPs and 15 CFPs, where there is one polyp tissue (or cancer as a separate analysis) per each patient within the groups; there were not multiple tissue types per patient included in the CAP or CFP groups. A difference was considered significant if the p-value was <0.05 or False Discovery Rate less than 0.1. Boxplots, bar graphs, and density plots were processed in R 2.15.1 as described elsewhere (see, e.g., RDC, “A language and environment for statistical computing,” R Foundation for Statistical Computing (2010)). Comparisons between the CAP and CFP tissues were done using the “edgeR” Library in R utilizing the offset from the CQN normalization and the tagwise dispersion estimate. Pearson's correlations are reported, unless otherwise stated, as described elsewhere (see, e.g., Robinson et al., Bioinformatics 26:139-140 (2010)). All the statistical analyses were performed using R software, unless otherwise stated. Heatmaps or clustering plots were generated using default parameters using the heatmap and hclust functions in R. The distances between samples for the CNV analysis and enrichment analyses for gene sets against KEGG pathways were calculated by −log p-value of the hypergeometric test.

### Data Availability

The raw data in BAM file format for the WGS, RNA-seq and RRBS data analyzed in this manuscript are available in the dbGaP database with Study Accession number: phs001384.v1.p1. The study report page can be accessed at:

www.ncbi.nlm.nih.gov/projects/gap/cgi-bin/study.cgi?study_id=phs001384.v1.p1 Accession numbers for each WGS, RNA-seq and RRBS BAM file are in Table 15 (FIG. 23).

### Example 2: Molecular Characterization of Colorectal Adenomas with and without Malignancy Reveals Distinguishing Genome, Transcriptome and Methylome Alterations

Whole Genome Sequencing and RNA-sequencing were performed (as described in Example 1) on polyps that were histologically and morphologically identical, but differ in that they have been removed and never recurred (POP-NR), recurred but were either cured via colonoscopy or surgery (POP-R), or recurred and presented with colorectal cancer (POP-CRC).

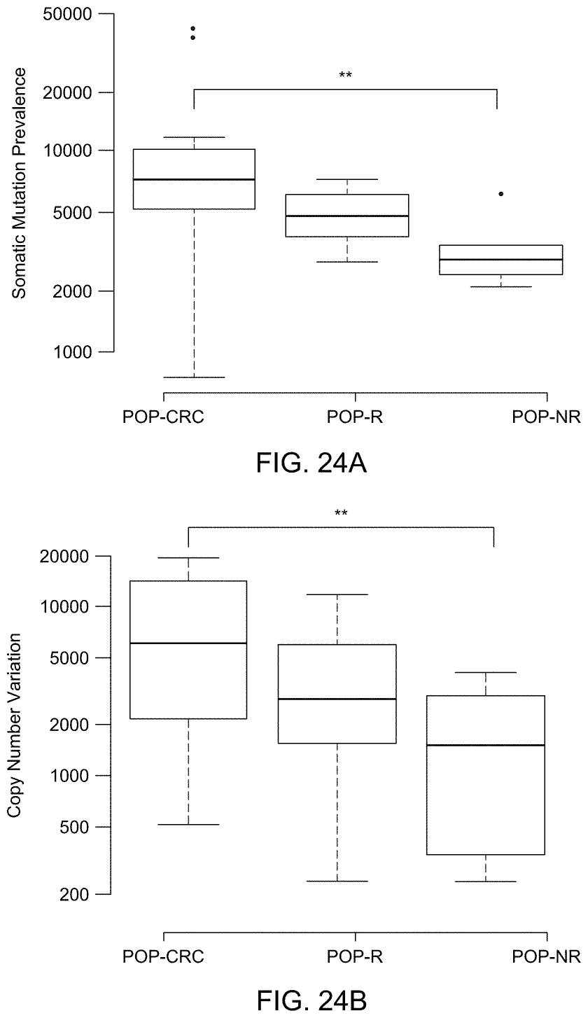

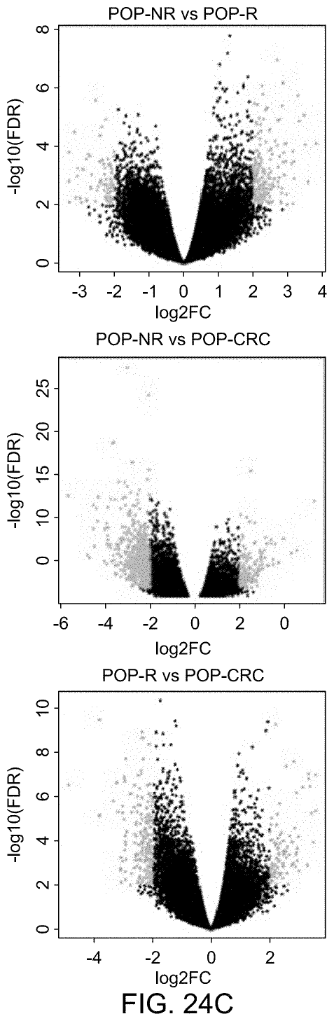

Whole Genome Sequencing and RNA-sequencing data indicated that somatic mutation prevalence (FIG. 24A), copy number variation (FIG. 24B), and gene expression (FIG. 24C) differed in POP-NR, POP-R, and POP-CRC polyps. For all comparisons, there were genes that overlap between the POP categories and genes that were unique to each category, with the most significant difference between the polyp tissues belonging to the POP-NR and POP-CRC categories.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having one or more colon polyps, wherein said method comprises:
(a) identifying at least one polyp from the mammal as having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF8, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, wherein said one or more modifications are selected from the group consisting of a somatic variation in a nucleic acid sequence, altered expression of a nucleic acid sequence, altered methylation of a nucleic acid sequence, and combinations thereof, and
(b) administering a colon polyp treatment to said mammal under conditions wherein the number of colon polyps within said mammal is reduced.

2. A method for treating a mammal having one or more colon polyps, wherein said method comprises:
administering a colon polyp treatment to a mammal identified as having at least one polyp having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG, RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, wherein said one or more modifications are selected from the group consisting of a somatic variation in a nucleic acid sequence, altered expression of a nucleic acid sequence, altered methylation of a nucleic acid sequence, and combinations thereof.

3. The method of claim 1, wherein said mammal is a human.

4. The method of claim 1, wherein said molecular profile comprises:
one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence;
increased expression of one or more of a CXCL5, a GREM1, an IGF2, a CTGF, a PLAU, and an E2F8 nucleic acid sequence;
reduced expression of an E2F8 nucleic acid sequence; and
hypermethylation of one or more a FES, a HES1, an ERBB3, and an E2F8 nucleic acid sequence.

5. The method of claim 1, wherein said molecular profile comprises:
one or more somatic variations in a TP53, a FBXW7, a PIK3CA, a KIAA1804, a SMAD2, and a SMAD4 nucleic acid sequence;
increased expression of a CXCL5, a GREM1, an IGF2, a CTGF, and a PLAU nucleic acid sequence;
reduced expression of an ERBB3 nucleic acid sequence; and
hypermethylation of a FES, a HES1, an ERBB3, and an E2F8 nucleic acid sequence.

6. The method of claim 1, wherein said colon polyp treatment comprises removal of one or more polyp(s) in addition to said polyp having said molecular profile.

7. The method of claim 1, said method further comprising selecting said mammal for more frequent cancer screening than was performed previously on said mammal.

8. The method of claim 7, said method further comprising performing said more frequent cancer screening.

9. The method of claim 7, wherein said cancer screening is selected from the group consisting of colonoscopy, barium enema x-rays, digital rectal examinations, and combinations thereof.

10. A method for treating a mammal having one or more colon polyps, wherein said method comprises:
(a) identifying at least one polyp from the mammal as having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, wherein said one or more modifications are selected from the group consisting of a somatic variation in a nucleic acid sequence, altered expression of a nucleic acid sequence, altered methylation of a nucleic acid sequence, and combinations thereof,
(b) administering a colon polyp treatment to said mammal under conditions wherein the number of colon polyps within said mammal is reduced; and
(c) administering a cancer treatment to said mammal.

11. A method for treating a mammal having one or more colon polyps, wherein said method comprises:
administering a colon polyp treatment to a mammal identified as having at least one polyp having a molecular profile comprising one or more modifications in one or more nucleic acid sequences selected from the group consisting of E2F8, COL2A1, GREM1, COL6A3, SCARF2, STK33, ERBB3, P2RY6, IGSF22, CNTN4, EFNB3, ZNF579, NEB, HES1, STX8, NUP210, MEGF10, GPC1, KIAA0825, GRIN2C, BRSK2, ARIH2, SATB1, SCN5A, PPARG, RARG, SOCS3, HHIP, RGMA, ANKRD36, NPC1L1, TNNC2, PRKACB, MED7, ZNF141, ALPPL2, TRRAP, TK1, C11orf63, RIMS2, BCL2L10, C4orf33, GYLTL1B, C1orf86, ZNF480, TAF1L, GBGT1, SST, FBN1, EBF4, NPW, TNC, FGF18, COG6, NOX5, ZNF470, PLXDC1, ATHL1, SNCAIP, IGF2, KMT2B, CRYBA2, IL11, CD248, NACAD, ACSL6, A1BG, CABP7, THRB, NUAK1, MATK, FARP1, CACNA1I, TRPC1, LYL1, RPH3A, KCNN2, CLYBL, SLITRK2, AHSA2, CHRD, CIT, DPY19L2P2, IGDCC3, COL12A1, HEBP1, COL4A3, ISLR, DNAH9, CDH3, ST6GALNAC5, ZNF599, GPRIN2, TANC2, SPEG, RASAL3, HMCN1, TRPV3, CR2, OTOP3, COL13A1, CPLX1, DUSP2, MT1JP, NOTCH3, ZNF726, ROBO3, CCK, SLC5A7, TSPY26P, FADS1, PLEKHG2, CACNA1H, LILRA1, COL5A2, ZNF836, FES, RIMS1, VANGL2, MUC4, BAIAP3, PLEKHH2, GPR98, COL11A2, APC, TP53, TTN, KRAS, FBXW7, PIK3CA, CTNNB1, KIAA1804, SMAD2, SMAD4, CXCL5, GREM1, IGF2, CTGF, PLAU, FES, HES1, ERBB3, and E2F8 nucleic acid sequences, wherein said one or more modifications are selected from the group consisting of a somatic variation in a nucleic acid sequence, altered expression of a nucleic acid sequence, altered methylation of a nucleic acid sequence, and combinations thereof, and
administering a cancer treatment to said mammal.

12. The method of claim 10, wherein said mammal is a human.

13. The method claim 10, wherein said molecular profile comprises:
one or more somatic variations in one or more of a APC, a TP53, a TTN, a KRAS, a FBXW7, a PIK3CA, a CTNNB1, a KIAA1804, a SMAD2, a SMAD4, an ERBB3, and an E2F8 nucleic acid sequence;
increased expression of one or more of a CXCL5, a GREM1, an IGF2, a CTGF, a PLAU, and an E2F8 nucleic acid sequence;
reduced expression of an E2F8 nucleic acid sequence; and
hypermethylation of one or more a FES, a HES1, an ERBB3, and an E2F8 nucleic acid sequence.

14. The method of claim 10, wherein said molecular profile comprises:
one or more somatic variations in a TP53, a FBXW7, a PIK3CA, a KIAA1804, a SMAD2, and a SMAD4 nucleic acid sequence;
increased expression of a CXCL5, a GREM1, an IGF2, a CTGF, and a PLAU nucleic acid sequence;
reduced expression of an ERBB3 nucleic acid sequence; and
hypermethylation of a FES, a HES1, an ERBB3, and an E2F8 nucleic acid sequence.

15. The method of claim 10, wherein said colon polyp treatment comprises removal of one or more polyp(s) in addition to said polyp having said molecular profile.

16. The method of claim 10, wherein said cancer treatment comprises administering a cancer drug to said mammal.

17. The method of claim 16, wherein said cancer drug is selected from the group consisting of capecitabine, fluorouracil, oxaliplatin, leucovorin, avastin, cetuximab, pembrolizumab, and combinations thereof.
